(19) United States
(12) Patent Application Publication
(10) Pub. No.: US 2025/0344602 A1
(43) Pub. Date: Nov. 6, 2025

(54) ORGANIC COMPOUND, LIGHT-EMITTING DEVICE INCLUDING THE SAME, AND ELECTRONIC APPARATUS INCLUDING THE LIGHT-EMITTING DEVICE

(71) Applicant: Samsung Display Co., Ltd., Yongin-si (KR)

(72) Inventors: Hyejeong Park, Yongin-si (KR); Sunmi Choi, Yongin-si (KR); Tomokatsu Kushida, Chiyoda-ku (JP); Yuki Nakano, Chiyoda-ku (JP); Takeshi Ikeda, Chiyoda-ku (JP); Hirokatsu Ito, Chiyoda-ku (JP)

(21) Appl. No.: 19/189,996

(22) Filed: Apr. 25, 2025

(30) Foreign Application Priority Data
May 3, 2024 (KR) 10-2024-0059422

(51) Int. Cl. H10K 85/60; C07D 209/08; C09K 11/02; H10K 50/115; H10K 50/12; H10K 101/20

(52) U.S. Cl. CPC H10K 85/626; C07D 209/08; C09K 11/02; H10K 85/615; H10K 85/6572; H10K 85/6574; H10K 50/115; H10K 50/12; H10K 2101/20

(57) ABSTRACT

A light-emitting device includes a first electrode, a second electrode opposite to the first electrode, and an interlayer between the first electrode and the second electrode and including an emission layer, wherein the interlayer includes an organic compound represented by Formula 1:

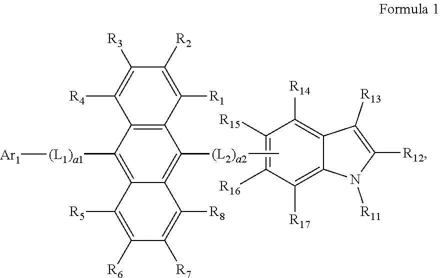

in Formula 1, one selected from among R₁₄ to R₁₇ indicating a binding site to (L₂)ₐ₂.

### CROSS-REFERENCE TO RELATED APPLICATION

The present application claims priority to and the benefit of Korean Patent Application No. 10-2024-0059422, filed on May 3, 2024, in the Korean Intellectual Property Office, the entire disclosure of which is incorporated herein by reference.

### BACKGROUND

### 1. Field

One or more embodiments of the present disclosure relate to an organic compound, a light-emitting device including the organic compound, and an electronic apparatus including the light-emitting device.

### 2. Description of the Related Art

A light-emitting device (e.g., an organic light-emitting device) may include a first electrode, a hole transport region, an emission layer, an electron transport region, and a second electrode that are sequentially arranged. Holes injected from the first electrode may move toward the emission layer through the hole transport region. Similarly, electrons injected from the second electrode may move toward the emission layer through the electron transport region. In the emission layer, carriers, such as these holes and electrons, recombine to produce excitons. As these excitons transition (decay) from an excited state to a ground state, they emit light, which may be used to display an image.

### SUMMARY

One or more aspects of embodiments of the present disclosure are directed toward an organic compound that has high stability and high hole-transporting ability and may be easily synthesized, a light-emitting device having improved external quantum efficiency by including the organic compound, and an electronic apparatus including the light-emitting device.

Additional aspects will be set forth in part in the description which follows and, in part, will be apparent from the description, or may be learned by practice of the presented embodiments of the disclosure.

According to one or more embodiments of the present disclosure, a light-emitting device includes a first electrode, a second electrode opposite to (e.g., facing) the first electrode, and an interlayer between the first electrode and the second electrode and including an emission layer, wherein the interlayer includes a first organic compound represented by Formula 1:

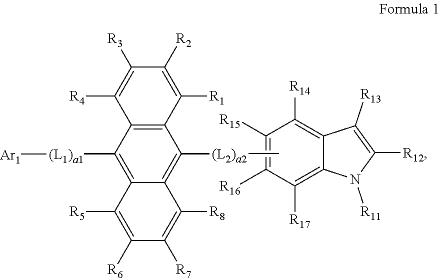

wherein, in Formula 1,
Ar₁ may be a C₃-C₆₀ carbocyclic group unsubstituted or substituted with at least one R₁₀ₐ or a C₁-C₆₀ heterocyclic group unsubstituted or substituted with at least one R₁₀ₐ,
L₁ and L₂ may each independently be a C₆-C₆₀ arylene group unsubstituted or substituted with at least one R₁₀ₐ or a C₁-C₆₀ heteroarylene group unsubstituted or substituted with at least one R₁₀ₐ,
a1 and a2 may each be an integer from 0 to 2,
if (e.g., when) a1 is 0, (L₁)ₐ₁ may be a single bond, and if (e.g., when) a2 is 0, (L₂)ₐ₂ may be a single bond,
R₁ to R₈ and R₁₁ to R₁₇ may each independently be hydrogen, deuterium, tritium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), provided that one selected from among R₁₄ to R₁₇ is (or indicates) a binding site to (L₂)ₐ₂,
R₁₀ₐ may be:
deuterium, tritium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, tritium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, tritium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), wherein
Q₁ to Q₃, Q₁₁ to Q₁₃, Q₂₁ to Q₂₃, and Q₃₁ to Q₃₃ may each independently be: hydrogen; deuterium; tritium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, tritium, —F, a cyano group, a C₁-C₆₀ alkyl group, a C₁-C₆₀ alkoxy group, a phenyl group, a biphenyl group, or any combination thereof.

According to one or more embodiments of the present disclosure, an electronic apparatus includes the light-emitting device and a thin-film transistor electrically connected to the light-emitting device.

According to one or more embodiments of the present disclosure, electronic equipment includes the electronic apparatus, wherein the electronic equipment may be at least one of a flat panel display, a curved display, a computer monitor, a medical monitor, a television, a billboard, a light for indoor lighting, a light for outdoor lighting, a light for signaling, a head-up display, a fully transparent display, a partially transparent display, a flexible display, a rollable display, a foldable display, a stretchable display, a laser printer, a telephone, a mobile phone, a tablet personal computer, a phablet, a personal digital assistant (PDA), a wearable device, a laptop computer, a digital camera, a camcorder, a viewfinder, a micro display, a 3D display, a virtual reality display, an augmented reality display, a vehicle, a video wall including multiple displays tiled together, a theater screen, a stadium screen, a phototherapy device, or a signboard.

According to one or more embodiments of the present disclosure, provided is the organic compound represented by Formula 1.

### BRIEF DESCRIPTION OF THE DRAWINGS

The accompanying drawings are included to provide a further understanding of the present disclosure and are incorporated in and constitute a part of the present disclosure. The drawings illustrate embodiments of the present disclosure and, together with the description, serve to explain principles of the present disclosure. The above and other aspects, features, and advantages of certain embodiments of the disclosure will be more apparent from the following description taken in conjunction with the accompanying drawings, in which:

FIG. 1 is a schematic view of a light-emitting device according to one or more embodiments of the present disclosure;

FIG. 2 is a schematic view of an electronic apparatus according to one or more embodiments of the present disclosure;

FIG. 3 is a schematic view of an electronic apparatus according to one or more embodiments of the present disclosure;

FIG. 4 is a schematic perspective view of electronic equipment including a light-emitting device according to one or more embodiments of the present disclosure;

FIG. 5 is a diagram schematically illustrating an exterior of a vehicle as electronic equipment including a light-emitting device according to one or more embodiments of the present disclosure; and

FIGS. 6A-6C are each a diagram schematically illustrating an interior of the vehicle of FIG. 5.

### DETAILED DESCRIPTION

Reference will now be made in more detail to one or more embodiments, examples of which are illustrated in the accompanying drawings, wherein like reference numerals refer to like elements throughout the present disclosure, and duplicative descriptions thereof may not be provided for conciseness. In this regard, the presented embodiments may have different forms and should not be construed as being limited to the descriptions set forth herein. Accordingly, the embodiments of the present disclosure are merely described, by referring to the drawings, to explain aspects of the present disclosure. As used herein, the term “and/or” or “or” may include any and all combinations of one or more of the associated listed items. Throughout the disclosure, the expressions such as “at least one of,” “one of,” and “selected from,” when preceding a list of elements, modify the entire list of elements and do not modify the individual elements of the list. For example, “at least one of a, b, or c,” “at least one selected from a, b, and c,” “at least one selected from among a to c,” etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

According to one or more embodiments of the present disclosure, a light-emitting device includes: a first electrode; a second electrode opposite to (e.g., facing) the first electrode; and an interlayer between the first electrode and the second electrode and including an emission layer, wherein the interlayer includes a first organic compound represented by Formula 1:

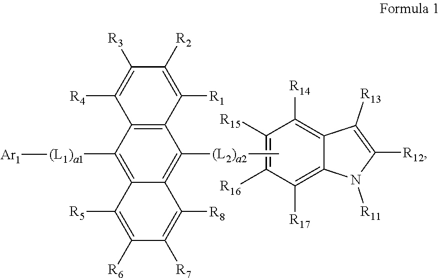

wherein, in Formula 1,
Ar₁ may be a C₃-C₆₀ carbocyclic group unsubstituted or substituted with at least one R₁₀ₐ or a C₁-C₆₀ heterocyclic group unsubstituted or substituted with at least one R₁₀ₐ,
L₁ and L₂ may each independently be a C₆-C₆₀ arylene group unsubstituted or substituted with at least one R₁₀ₐ or a C₁-C₆₀ heteroarylene group unsubstituted or substituted with at least one R₁₀ₐ,
a1 and a2 may each be an integer from 0 to 2,
if (e.g., when) a1 is 0, (L₁)ₐ₁ may be a single bond, and if (e.g., when) a2 is 0, (L₂)ₐ₂ may be a single bond,
R₁ to R₈ and R₁₁ to R₁₇ may each independently be hydrogen, deuterium, tritium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), provided that one selected from among R₁₄ to R₁₇ is (or indicates) a binding site to (L₂)ₐ₂,
R₁₀ₐ may be:
deuterium, tritium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, tritium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, tritium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium, tritium; —F; —Cl; —Br; —I; a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group; or a C₃-C₆₀ carbocyclic group or a C₁-C₆₀ heterocyclic group, each unsubstituted or substituted with deuterium, tritium, —F, a cyano group, a C₁-C₆₀ alkyl group, a C₁-C₆₀ alkoxy group, a phenyl group, a biphenyl group, or any combination thereof.

The first electrode of the light-emitting device may be an anode, and the second electrode of the light-emitting device may be a cathode.

In one or more embodiments, the interlayer may further include a hole transport region between the first electrode and the emission layer and an electron transport region between the emission layer and the second electrode. The hole transport region may include a hole injection layer, a hole transport layer, an emission auxiliary layer, an electron blocking layer, or any combination thereof. The electron transport region may include a buffer layer, a hole blocking layer, an electron control layer, an electron transport layer, an electron injection layer, or any combination thereof.

In one or more embodiments, the emission layer may include the first organic compound. For example, the first organic compound may serve as a host. In one or more embodiments, the interlayer may further include a dopant that emits fluorescence or delayed fluorescence. For example, the emission layer may further include a dopant that emits fluorescence or delayed fluorescence. The emission layer may be to emit red light, green light, blue light, and/or white light (e.g., combined white light). In one or more embodiments, the emission layer may be to emit blue light. For example, the emission layer may be to emit light having a maximum emission wavelength in a range of about 400 nanometers (nm) to about 490 nm.

In one or more embodiments, the interlayer may further include a second organic compound that is represented by Formula 2 and is different from the first organic compound:

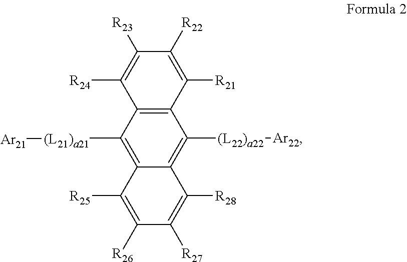

wherein, in Formula 2,
Ar₂₁ and Ar₂₂ may each independently be a C₃-C₆₀ carbocyclic group unsubstituted or substituted with at least one R_{10b} or a C₁-C₆₀ heterocyclic group unsubstituted or substituted with at least one R_{10b},
L₂₁ and L₂₂ may each independently be a C₆-C₆₀ arylene group unsubstituted or substituted with at least one R_{10b} or a C₁-C₆₀ heteroarylene group unsubstituted or substituted with at least one R_{10b},
a21 and a22 may each be an integer from 0 to 2,
if (e.g., when) a21 is 0, (L₂₁)ₐ₂₁ may be a single bond, and if (e.g., when) a22 is 0, (L₂₂)ₐ₂₂ may be a single bond,
R₂₁ to R₂₈ may each independently be hydrogen, deuterium, tritium, —F, —Cl, —Br, —I, a hydroxyl group, a cyano group, a nitro group, a C₁-C₆₀ alkyl group unsubstituted or substituted with at least one R_{10b}, a C₂-C₆₀ alkenyl group unsubstituted or substituted with at least one R_{10b}, a C₂-C₆₀ alkynyl group unsubstituted or substituted with at least one R_{10b}, a C₁-C₆₀ alkoxy group unsubstituted or substituted with at least one R_{10b}, a C₁-C₆₀ alkylthio group unsubstituted or substituted with at least one R_{10b}, a C₃-C₆₀ carbocyclic group unsubstituted or substituted with at least one R_{10b}, a C₁-C₆₀ heterocyclic group unsubstituted or substituted with at least one R_{10b}, a C₆-C₆₀ aryloxy group unsubstituted or substituted with at least one R_{10b}, a C₆-C₆₀ arylthio group unsubstituted or substituted with at least one R_{10b}, —Si(Q₄₁)(Q₄₂)(Q₄₃), —N(Q₄₁)(Q₄₂), —B(Q₄₁)(Q₄₂), —C(═O)(Q₄₁), —S(═O)₂(Q₄₁), or —P(═O)(Q₄₁)(Q₄₂),
R_{10b} may be:
deuterium, tritium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, tritium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₅₁)(Q₅₂)(Q₅₃), —N(Q₅₁)(Q₅₂), —B(Q₅₁)(Q₅₂), —C(═O)(Q₅₁), —S(═O)₂(Q₅₁), —P(═O)(Q₅₁)(Q₅₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, tritium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₆₁)(Q₆₂)(Q₆₃), —N(Q₆₁)(Q₆₂), —B(Q₆₁)(Q₆₂), —C(═O)(Q₆₁), —S(═O)₂(Q₆₁), —P(═O)(Q₆₁)(Q₆₂), or any combination thereof; or
—Si(Q₇₁)(Q₇₂)(Q₇₃), —N(Q₇₁)(Q₇₂), —B(Q₇₁)(Q₇₂), —C(═O)(Q₇₁), —S(═O)₂(Q₇₁), or —P(═O)(Q₇₁)(Q₇₂), and

Q₄₁ to Q₄₃, Q₅₁ to Q₅₃, Q₆₁ to Q₆₃, and Q₇₁ to Q₇₃ may each independently be: hydrogen; deuterium, tritium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, tritium, —F, a cyano group, a C₁-C₆₀ alkyl group, a C₁-C₆₀ alkoxy group, a phenyl group, a biphenyl group, or any combination thereof.

In one or more embodiments, Ar₂₁ in Formula 2 may be a C₆-C₆₀ aryl group unsubstituted or substituted with at least one R_{10b}. For example, Ar₂₁ may be a phenyl group that is unsubstituted or substituted with at least one R_{10b} or a naphthalenyl group that is unsubstituted or substituted with at least one Rob.

Ar₂₂ may be a C₆-C₆₀ aryl group that is unsubstituted or substituted with at least one R_{10b} or a monovalent non-aromatic condensed heteropolycyclic group that is unsubstituted or substituted with at least one R_{10b}. For example, Ar₂₂ may be a phenyl group, a naphthalenyl group, a dibenzofuranyl group, a dibenzothiophenyl group, or a benzonaphthofuranyl group, each unsubstituted or substituted with at least one R_{10b}.

If (e.g., when) a21 is 2, two L₂₁(s) may be identical to or different from each other. If (e.g., when) a22 is 2, two L₂₂(s) may be identical to or different from each other.

In one or more embodiments, a21 in Formula 2 may be 0.

In one or more embodiments, in Formula 2, i) if (e.g., when) a22 is 0, Ar₂₂ may be a C₆-C₆₀ aryl group that is unsubstituted or substituted with at least one R_{10b}, or ii) if (e.g., when) a22 is 0, Ar₂₂ may be a monovalent non-aromatic condensed heteropolycyclic group that is unsubstituted or substituted with at least one R_{10b}.

In one or more embodiments, in Formula 2, L₂₁ and L₂₂ may each independently be a phenylene group that is unsubstituted or substituted with at least one R_{10b}. For example, in one or more embodiments, L₂₁ and L₂₂ may each independently be an ortho-phenylene group, a meta-phenylene group, or a para-phenylene group, each unsubstituted or substituted with at least one Rob.

In one or more embodiments, the second organic compound may include at least one deuterium, but embodiments of the present disclosure are not limited thereto.

In one or more embodiments, the emission layer may include both (e.g., simultaneously) the first organic compound and the second organic compound. A weight ratio of the first organic compound to the second organic compound may be in a range of about 1:10 to about 10:1, for example, about 1:5 to about 5:1, about 1:4 to about 4:1, or about 1:3 to about 3:1.

According to one or more embodiments of the present disclosure, an electronic apparatus may include: the light-emitting device; and a thin-film transistor electrically connected to the light-emitting device.

According to one or more embodiments of the present disclosure, an electronic equipment may include the electronic apparatus, wherein the electronic equipment may be at least one of a flat panel display, a curved display, a computer monitor, a medical monitor, a television, a billboard, a light for indoor lighting, a light for outdoor lighting, a light for signaling, a head-up display, a fully transparent display, a partially transparent display, a flexible display, a rollable display, a foldable display, a stretchable display, a laser printer, a telephone, a mobile phone, a tablet personal computer, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall including multiple displays tiled together, a theater screen, a stadium screen, a phototherapy device, or a signboard.

According to one or more embodiments of the present disclosure, provided is the organic compound represented by Formula 1.

In Formula 1, one selected from among R₁₄ to R₁₇ indicates a binding site to (L₂)ₐ₂, and thus a compound in which one selected from among R₁₁ to R₁₃ indicates a binding site to (L₂)ₐ₂ is clearly distinguished from the organic compound according to one or more embodiments of the present disclosure. In other words, a compound where one selected from among R₁₁ to R₁₃ is or serves as a binding site to (L₂)ₐ₂ is clearly distinct from the organic compound described in one or more embodiments of the present disclosure.

In Formula 1, an indole group may be linked to a carbon atom at the position 9 (or 10) of an anthracene group through (L₂)ₐ₂ to which the binding site indicated by one selected from R₁₄ to R₁₇ is linked. Thus, a compound in which an indole group is linked to a carbon atom atone of the positions 1 to 8 of an anthracene group is clearly distinguished from the organic compound according to one or more embodiments of the present disclosure. Because the indole group is linked to a carbon atom at the position 9 (or 10) of the anthracene group, the organic compound may be easily synthesized.

Because the organic compound according to one or more embodiments includes the indole group, a compound including, instead of the indole group, a benzimidazole group, a benzoxazole group, or a benzothiazole group is clearly distinguished from the organic compound according to one or more embodiments of the present disclosure.

In one or more embodiments, in Formula 1, Ar₁ may be a C₆-C₆₀ aryl group or a C₁-C₆₀ heteroaryl group, each unsubstituted or substituted with deuterium, tritium, a cyano group, a C₁-C₆₀ alkyl group, a C₁-C₆₀ alkoxy group, or any combination thereof.

In one or more embodiments, in Formula 1, Ar₁ may be a phenyl group, a naphthalenyl group, an anthracenyl group, a phenanthrenyl group, a phenalenyl group, a tetracenyl group, a pyrenyl group, a chrysenyl group, a triphenylenyl group, a pentacenyl group, or a perylenyl group, each unsubstituted or substituted with at least one R₁₀ₐ.

In one or more embodiments, in Formula 1, L₁ and L₂ may each independently be a phenylene group that is unsubstituted or substituted with at least one R₁₀ₐ. For example, L₁ and L₂ may each independently be an ortho-phenylene group, a meta-phenylene group, or a para-phenylene group, each unsubstituted or substituted with at least one R₁₀ₐ.

In one or more embodiments, in Formula 1, i) a1 may be 0, or ii) L₁ may be a phenylene group unsubstituted or substituted with at least one R₁₀ₐ.

In one or more embodiments, in Formula 1, a2 may be 0.

In one or more embodiments, in Formula 1, R₁₁ to R₁₃ may each independently be hydrogen, deuterium, tritium, a C₁-C₆₀ alkyl group unsubstituted or substituted with at least one R₁₀ₐ, a C₆-C₆₀ aryl group unsubstituted or substituted with at least one R₁₀ₐ, or a C₁-C₆₀ heteroaryl group unsubstituted or substituted with at least one R₁₀ₐ.

In one or more embodiments, in Formula 1, at least one of R₁₂ or R₁₃ may be a C₆-C₆₀ aryl group that is unsubstituted or substituted with at least one R₁₀ₐ or a C₁-C₆₀ heteroaryl group that is unsubstituted or substituted with at least one R₁₀ₐ.

For example, in one or more embodiments, R₁₁ may be a phenyl group that is unsubstituted or substituted with at least one deuterium or a naphthalenyl group that is unsubstituted or substituted with at least one deuterium. At least one of R₁₂ or R₁₃ may be a phenyl group that is unsubstituted or substituted with at least one deuterium or a naphthalenyl group that is unsubstituted or substituted with at least one deuterium.

In one or more embodiments, the organic compound may be represented by Formula 1-1:

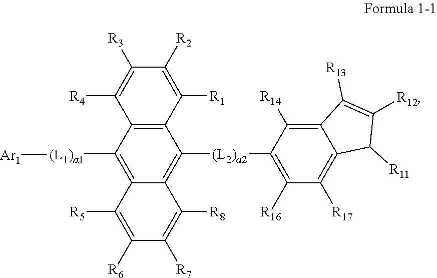

wherein, in Formula 1-1, Ar₁, L₁, L₂, a1, a2, R₁ to R₈, R₁₁ to R₁₄, R₁₆, and R₁₇ are each the same as described elsewhere herein. In other words, it is referred to that R₁₅ in Formula 1 may indicate a binding site to (L₂)ₐ₂.

In one or more embodiments, the organic compound may include at least one deuterium, but embodiments of the present disclosure are not limited thereto.

In one or more embodiments, the organic compound may satisfy at least one selected from among Conditions 1 to 8:

### Condition 1

Ar₁ is a phenyl group substituted with at least one deuterium;

### Condition 2

L₁ is a phenylene group substituted with at least one deuterium;

### Condition 3

at least one selected from among R₁ to R₈ is deuterium;

### Condition 4

L₂ is a phenylene group substituted with at least one deuterium;

### Condition 5

at least one selected from among R₁₄ to R₁₇ is deuterium;

### Condition 6

R₁₁ is selected from among deuterium, a phenyl group that is substituted with at least one deuterium, and a naphthalenyl group that is substituted with at least one deuterium;

### Condition 7

R₁₂ is selected from among deuterium, a phenyl group that is substituted with at least one deuterium, and a naphthalenyl group that is substituted with at least one deuterium; and

### Condition 8

R₁₃ is selected from among deuterium, a phenyl group that is substituted with at least one deuterium, and a naphthalenyl group that is substituted with at least one deuterium.

For example, the organic compound may satisfy one or more selected from among Conditions 1A to 8A:

### Condition 1A

Ar₁ is a phenyl group substituted with deuterium;

### Condition 2A

L₁ is a phenylene group substituted with deuterium;

### Condition 3A

each of R₁ to R₈ is deuterium;

### Condition 4A

L₂ is a phenylene group substituted with deuterium;

### Condition 5A

three of R₁₄ to R₁₇ are each deuterium;

### Condition 6A

R₁₁ is selected from among deuterium, a phenyl group that is substituted with deuterium, and a naphthalenyl group that is substituted with deuterium;

### Condition 7A

R₁₂ is selected from among deuterium, a phenyl group that is substituted with deuterium, and a naphthalenyl group that is substituted with deuterium; and

### Condition 8A

R₁₃ is selected from among deuterium, a phenyl group that is substituted with deuterium, and a naphthalenyl group that is substituted with deuterium.

In one or more embodiments, the organic compound may not include (e.g., may exclude) a benzimidazole group, a benzoxazole group, a benzothiazole group, and a triphenylene group.

In one or more embodiments, the organic compound may be one of (e.g., any one selected from among) Compounds BH1 to BH54:

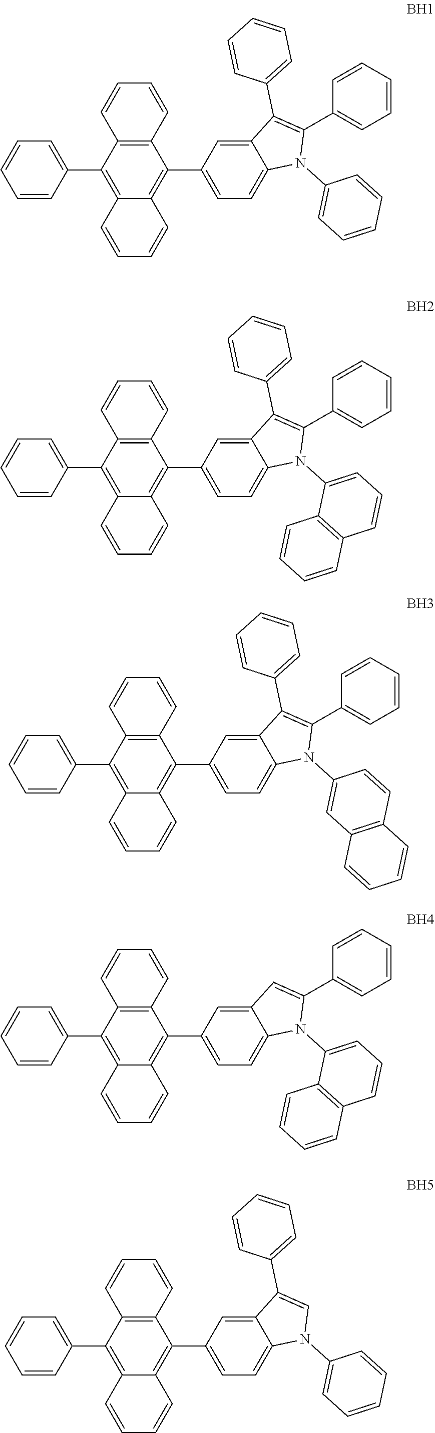

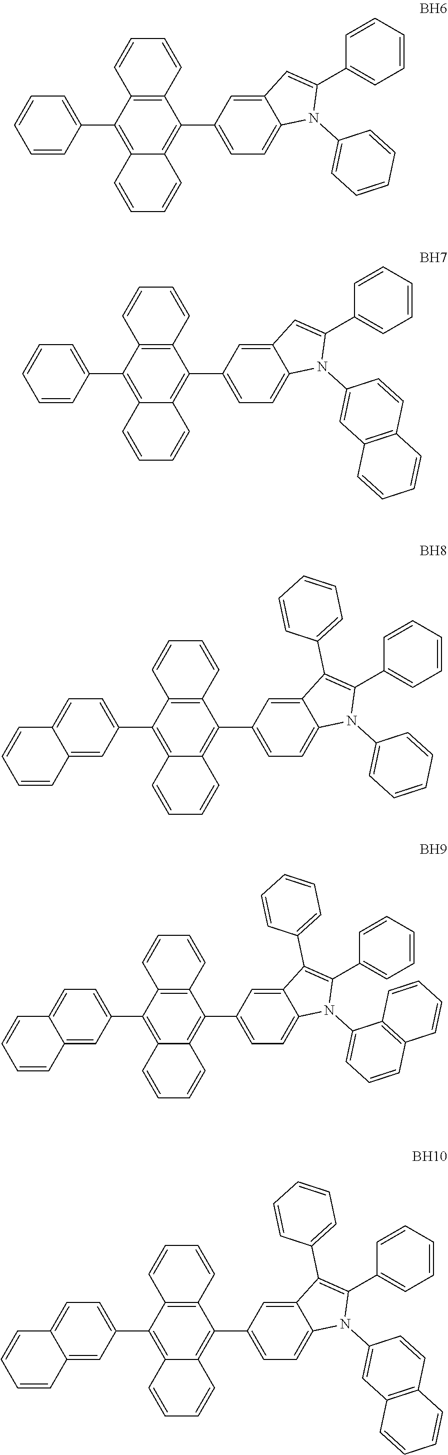 [SMILES: c1ccc(-c2c3ccccc3c(-c3ccc4c(c3)cc(-c3ccccc3)n4-c3ccccc3)c3ccccc23)cc1 ; c1ccc(-c2c3ccccc3c(-c3ccc4c(c3)cc(-c3ccccc3)n4-c3ccc4ccccc4c3)c3ccccc23)cc1 ; c1ccc(-c2c(-c3ccccc3)n(-c3ccccc3)c3ccc(-c4c5ccccc5c(-c5ccc6ccccc6c5)c5ccccc45)cc23)cc1 ; c1ccc(-c2c(-c3ccccc3)n(-c3cccc4ccccc34)c3ccc(-c4c5ccccc5c(-c5ccc6ccccc6c5)c5ccccc45)cc23)cc1 ; c1ccc(-c2c(-c3ccccc3)n(-c3ccc4ccccc4c3)c3ccc(-c4c5ccccc5c(-c5ccc6ccccc6c5)c5ccccc45)cc23)cc1]

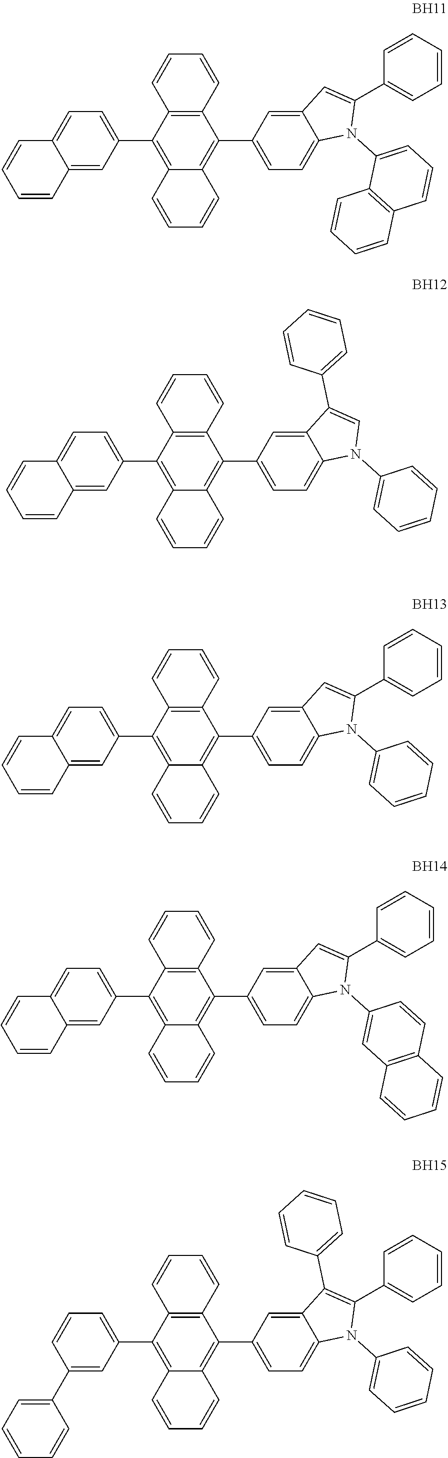 [SMILES: c1ccc(-c2cc3cc(-c4c5ccccc5c(-c5ccc6ccccc6c5)c5ccccc45)ccc3n2-c2cccc3ccccc23)cc1 ; c1ccc(-c2cn(-c3ccccc3)c3ccc(-c4c5ccccc5c(-c5ccc6ccccc6c5)c5ccccc45)cc23)cc1 ; c1ccc(-c2cc3cc(-c4c5ccccc5c(-c5ccc6ccccc6c5)c5ccccc45)ccc3n2-c2ccccc2)cc1 ; c1ccc(-c2cc3cc(-c4c5ccccc5c(-c5ccc6ccccc6c5)c5ccccc45)ccc3n2-c2ccc3ccccc3c2)cc1 ; c1ccc(-c2cccc(-c3c4ccccc4c(-c4ccc5c(c4)c(-c4ccccc4)c(-c4ccccc4)n5-c4ccccc4)c4ccccc34)c2)cc1]

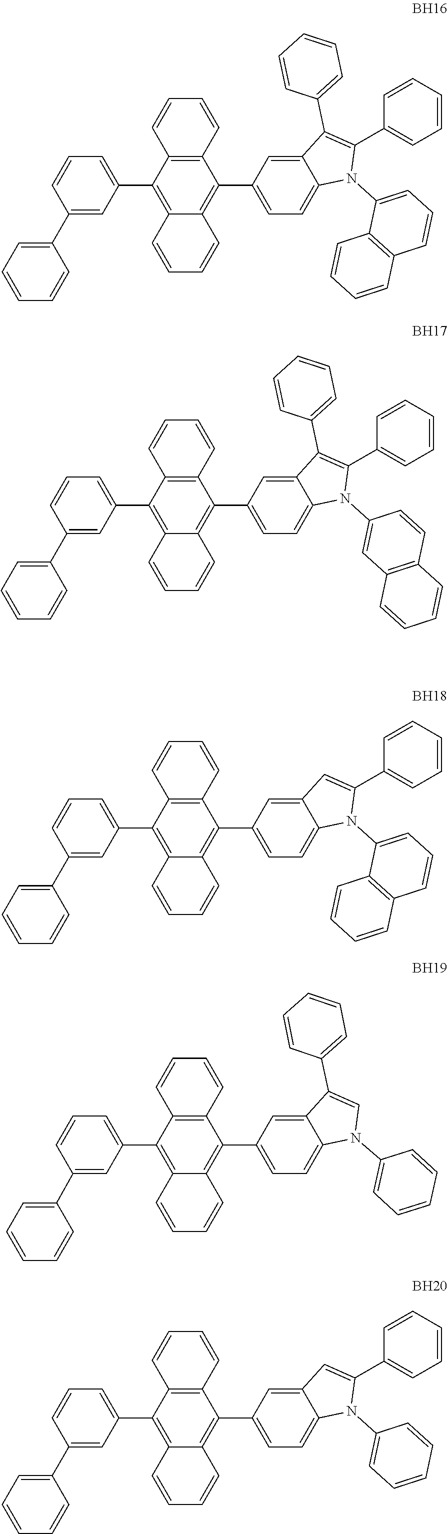 [SMILES: c1ccc(-c2cccc(-c3c4ccccc4c(-c4ccc5c(c4)c(-c4ccccc4)c(-c4ccccc4)n5-c4cccc5ccccc45)c4ccccc34)c2)cc1 ; c1ccc(-c2cccc(-c3c4ccccc4c(-c4ccc5c(c4)c(-c4ccccc4)c(-c4ccccc4)n5-c4ccc5ccccc5c4)c4ccccc34)c2)cc1 ; c1ccc(-c2cccc(-c3c4ccccc4c(-c4ccc5c(c4)cc(-c4ccccc4)n5-c4cccc5ccccc45)c4ccccc34)c2)cc1 ; c1ccc(-c2cccc(-c3c4ccccc4c(-c4ccc5c(c4)c(-c4ccccc4)cn5-c4ccccc4)c4ccccc34)c2)cc1 ; c1ccc(-c2cccc(-c3c4ccccc4c(-c4ccc5c(c4)cc(-c4ccccc4)n5-c4ccccc4)c4ccccc34)c2)cc1]

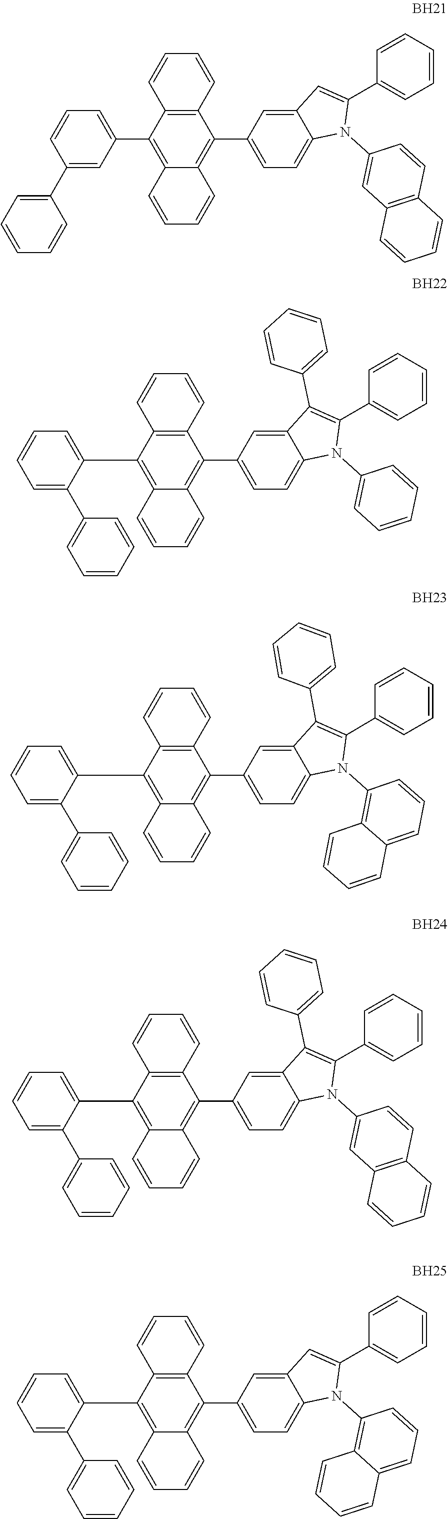 [SMILES: c1ccc(-c2cccc(-c3c4ccccc4c(-c4ccc5c(c4)cc(-c4ccccc4)n5-c4ccc5ccccc5c4)c4ccccc34)c2)cc1 ; c1ccc(-c2ccccc2-c2c3ccccc3c(-c3ccc4c(c3)c(-c3ccccc3)c(-c3ccccc3)n4-c3ccccc3)c3ccccc23)cc1 ; c1ccc(-c2ccccc2-c2c3ccccc3c(-c3ccc4c(c3)c(-c3ccccc3)c(-c3ccccc3)n4-c3cccc4ccccc34)c3ccccc23)cc1 ; c1ccc(-c2ccccc2-c2c3ccccc3c(-c3ccc4c(c3)c(-c3ccccc3)c(-c3ccccc3)n4-c3ccc4ccccc4c3)c3ccccc23)cc1 ; c1ccc(-c2ccccc2-c2c3ccccc3c(-c3ccc4c(c3)cc(-c3ccccc3)n4-c3cccc4ccccc34)c3ccccc23)cc1]

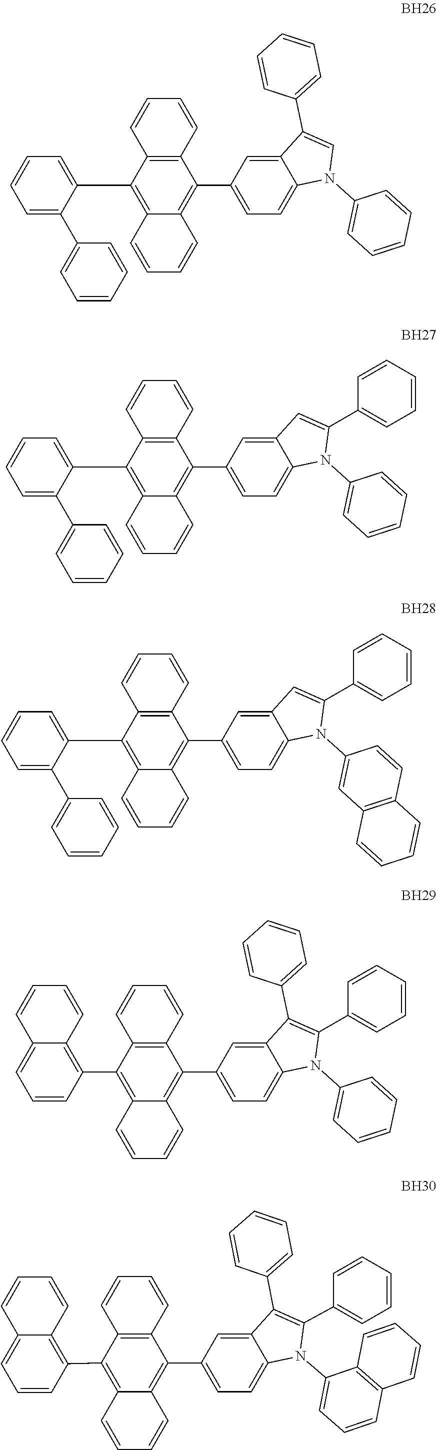 [SMILES: c1ccc(-c2ccccc2-c2c3ccccc3c(-c3ccc4c(c3)c(-c3ccccc3)cn4-c3ccccc3)c3ccccc23)cc1 ; c1ccc(-c2ccccc2-c2c3ccccc3c(-c3ccc4c(c3)cc(-c3ccccc3)n4-c3ccccc3)c3ccccc23)cc1 ; c1ccc(-c2ccccc2-c2c3ccccc3c(-c3ccc4c(c3)cc(-c3ccccc3)n4-c3ccc4ccccc4c3)c3ccccc23)cc1 ; c1ccc(-c2c(-c3ccccc3)n(-c3ccccc3)c3ccc(-c4c5ccccc5c(-c5cccc6ccccc56)c5ccccc45)cc23)cc1 ; c1ccc(-c2c(-c3ccccc3)n(-c3cccc4ccccc34)c3ccc(-c4c5ccccc5c(-c5cccc6ccccc56)c5ccccc45)cc23)cc1]

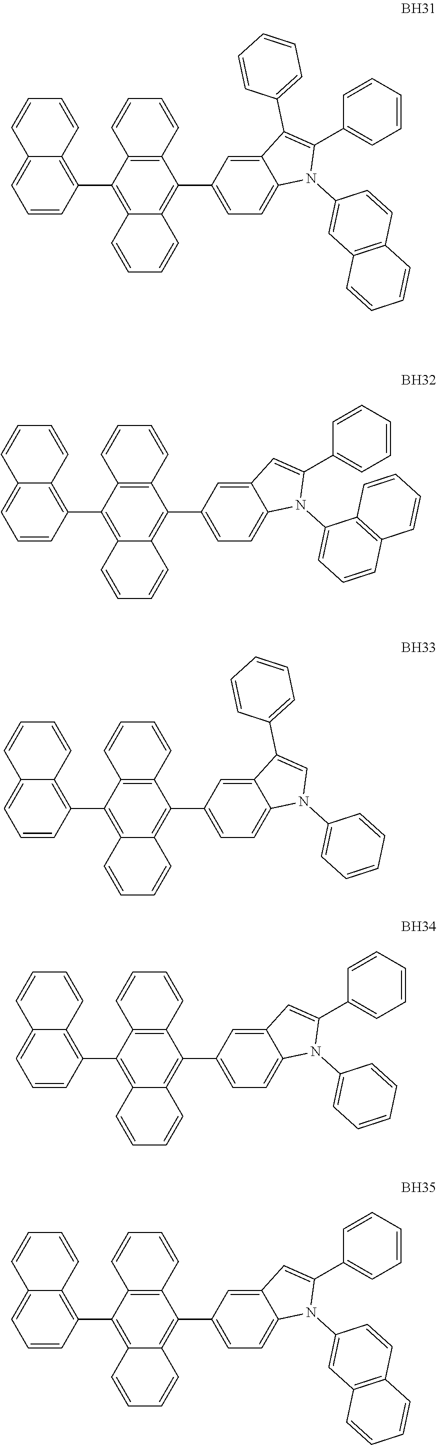 [SMILES: c1ccc(-c2c(-c3ccccc3)n(-c3ccc4ccccc4c3)c3ccc(-c4c5ccccc5c(-c5cccc6ccccc56)c5ccccc45)cc23)cc1 ; c1ccc(-c2cc3cc(-c4c5ccccc5c(-c5cccc6ccccc56)c5ccccc45)ccc3n2-c2cccc3ccccc23)cc1 ; c1ccc(-c2cn(-c3ccccc3)c3ccc(-c4c5ccccc5c(-c5cccc6ccccc56)c5ccccc45)cc23)cc1 ; c1ccc(-c2cc3cc(-c4c5ccccc5c(-c5cccc6ccccc56)c5ccccc45)ccc3n2-c2ccccc2)cc1 ; c1ccc(-c2cc3cc(-c4c5ccccc5c(-c5cccc6ccccc56)c5ccccc45)ccc3n2-c2ccc3ccccc3c2)cc1]

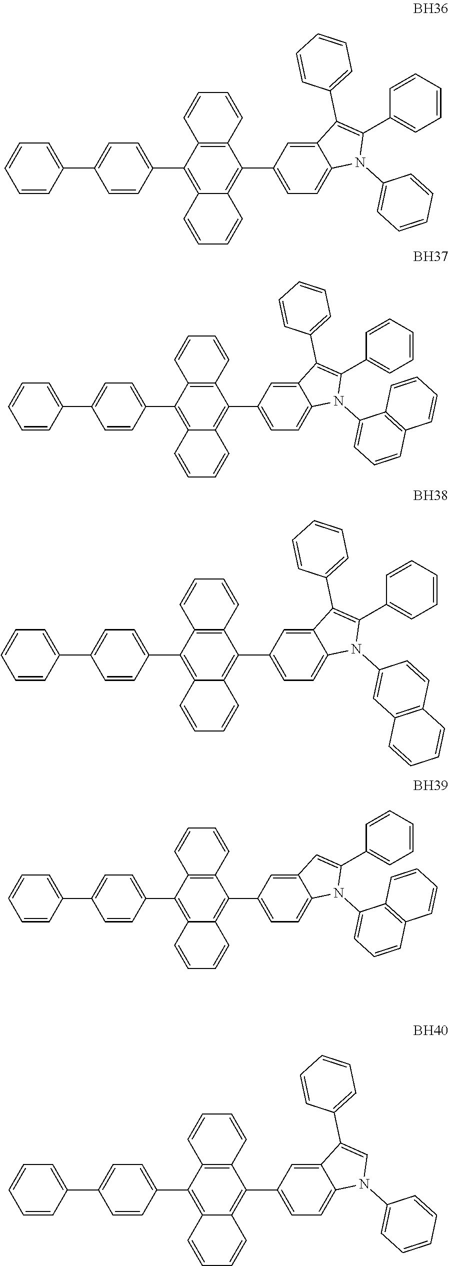 [SMILES: c1ccc(-c2ccc(-c3c4ccccc4c(-c4ccc5c(c4)c(-c4ccccc4)c(-c4ccccc4)n5-c4ccccc4)c4ccccc34)cc2)cc1 ; c1ccc(-c2ccc(-c3c4ccccc4c(-c4ccc5c(c4)c(-c4ccccc4)c(-c4ccccc4)n5-c4cccc5ccccc45)c4ccccc34)cc2)cc1 ; c1ccc(-c2ccc(-c3c4ccccc4c(-c4ccc5c(c4)c(-c4ccccc4)c(-c4ccccc4)n5-c4ccc5ccccc5c4)c4ccccc34)cc2)cc1 ; c1ccc(-c2ccc(-c3c4ccccc4c(-c4ccc5c(c4)cc(-c4ccccc4)n5-c4cccc5ccccc45)c4ccccc34)cc2)cc1 ; c1ccc(-c2ccc(-c3c4ccccc4c(-c4ccc5c(c4)c(-c4ccccc4)cn5-c4ccccc4)c4ccccc34)cc2)cc1]

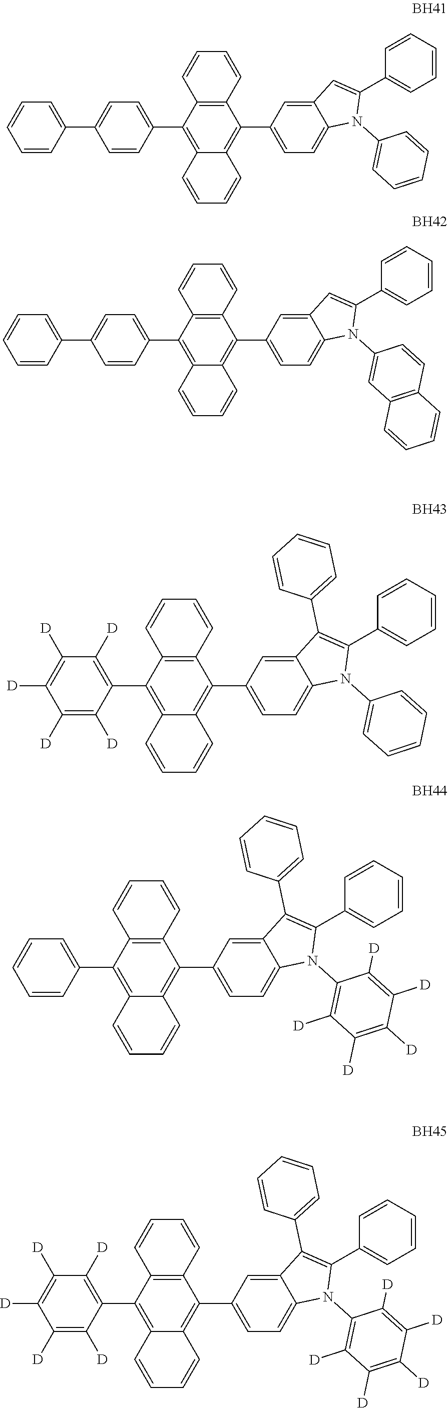

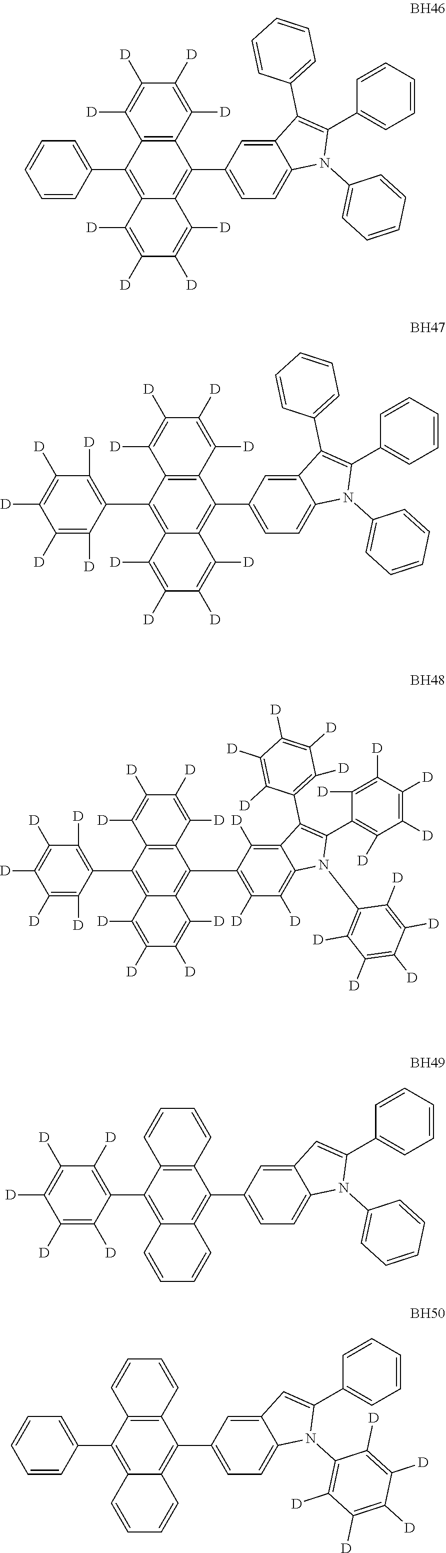

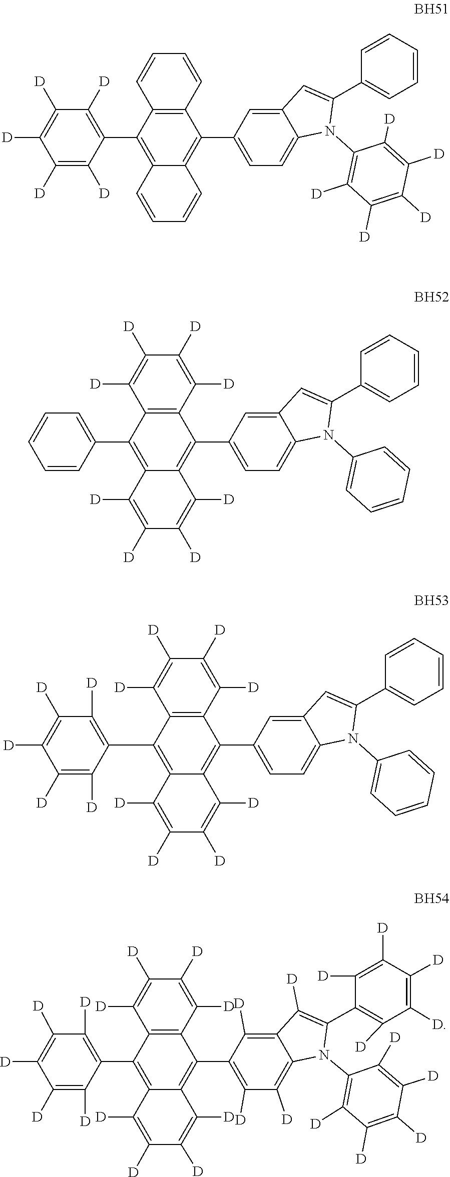

The organic compound represented by Formula 1 according to one or more embodiments may be a compound in which a carbon atom at position 9 (or 10) of the anthracene group is linked to a carbon atom atone of positions 4 to 7 of the indole group. The organic compound may serve as a blue host (i.e., a host for a dopant to emit blue light), and thus is clearly different from a red host or a green host.

The organic compound may have improved stability by including anthracene which has low triplet energy (T1) as compared to triphenylene.

The organic compound may have excellent or suitable hole-transporting ability by including indole rather than benzimidazole, benzoxazole, or benzothiazole.

The organic compound is not a compound in which anthracene is linked to a nitrogen atom at position 1, a carbon atom at position 2, or a carbon atom at position 3 of indole, but a compound in which anthracene is linked to a carbon atom atone of positions 4 to 7 of indole. In this regard, the organic compound may have an energy level suitable for application as a blue host, and a light-emitting device including the organic compound may have improved external quantum efficiency.

The carbon atoms at positions 1 to 8 of anthracene have relatively low reactivity, and therefore are unlikely to bond to indole. In contrast, the organic compound is a compound in which a carbon atom at position 9 (or 10) of anthracene having relatively high reactivity is bonded to indole, and thus may be easily synthesized.

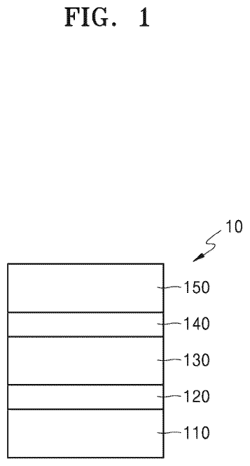

### Description of FIG. 1

FIG. 1 is a schematic cross-sectional view of a light-emitting device 10 according to one or more embodiments. The light-emitting device 10 includes a first electrode 110, an interlayer, and a second electrode 150. The interlayer may include a hole transport region 120, an emission layer 130, and an electron transport region 140. Hereinafter, a structure of the light-emitting device 10 according to one or more embodiments and a method of manufacturing the light-emitting device 10 will be described with reference to FIG. 1.

### First Electrode 110

In FIG. 1, in one or more embodiments, a substrate may be additionally provided and arranged under the first electrode 110 and/or on the second electrode 150. In one or more embodiments, as the substrate, a glass substrate or a plastic substrate may be used. The substrate may be a flexible substrate. For example, the substrate may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by providing a material for forming the first electrode 110 on the substrate by using a deposition method or a sputtering method. If (e.g., when) the first electrode 110 is an anode, a material for forming the first electrode 110 may be a high-work function material that facilitates injection of holes.

The first electrode 110 may be a reflective electrode, a semi-transmissive electrode, or a transmissive electrode. In one or more embodiments, if (e.g., when) the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, if (e.g., when) the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layer structure including (e.g., consisting of) a single layer or a multi-layer structure including multiple layers. For example, in some embodiments, the first electrode 110 may have a three-layer structure of ITO/Ag/ITO.

### Interlayer

The interlayer may be arranged on the first electrode 110. The interlayer may include the hole transport region 120, the emission layer 130, and the electron transport region 140.

The interlayer may include one or more suitable organic materials, a metal-containing compound such as an organometallic compound, an inorganic material such as quantum dots, and/or the like.

In one or more embodiments, the interlayer may include i) two or more light-emitting units sequentially stacked between the first electrode 110 and the second electrode 150, and ii) a charge generation layer between every two light-emitting units. When the interlayer includes the light-emitting units and the charge generation layer, the light-emitting device 10 may be a tandem light-emitting device.

### Hole Transport Region 120

The hole transport region 120 may have i) a single-layer structure including (e.g., consisting of) a single layer including a single material, ii) a single-layer structure including (e.g., consisting of) a single layer including multiple materials that are different from each other, or iii) a multi-layer structure including (e.g., consisting of) multiple layers including multiple materials that are different from each other.

The hole transport region 120 may include a hole injection layer, a hole transport layer, an emission auxiliary layer, an electron blocking layer, or any combination thereof.

For example, in one or more embodiments, the hole transport region 120 may have a multi-layer structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein constituent layers of each structure are stacked sequentially from the first electrode 110 in the stated order.

In one or more embodiments, the hole transport region may include a compound represented by Formula 201, a compound represented by Formula 202, or any combination thereof:

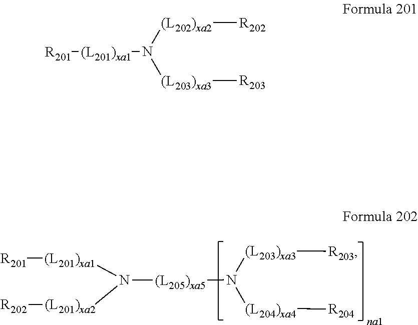

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (e.g., a carbazole group, and/or the like) unsubstituted or substituted with at least one R₁₀ₐ (e.g., see Compound HT16, and/or the like),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

For example, in one or more embodiments, each of Formulae 201 and 202 may include at least one selected from among groups represented by Formulae CY201 to CY217:

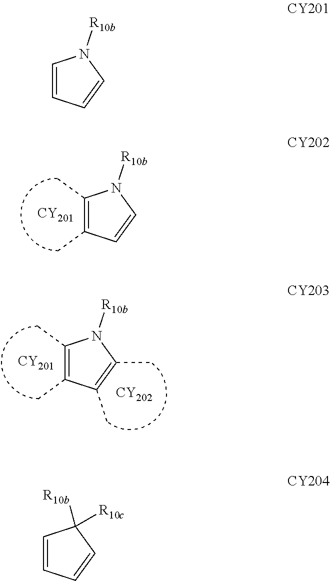

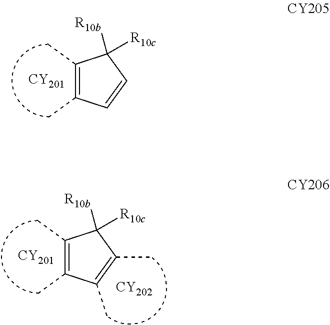

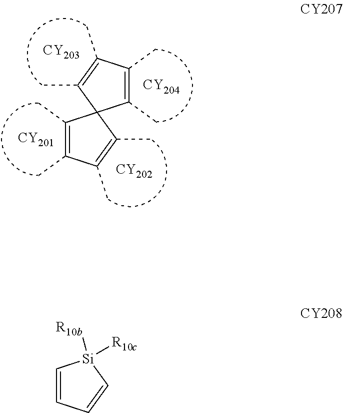

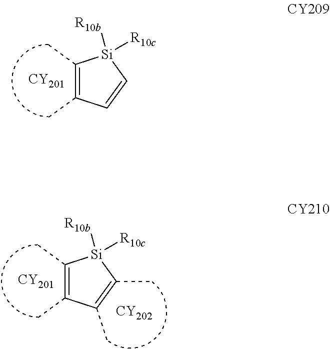

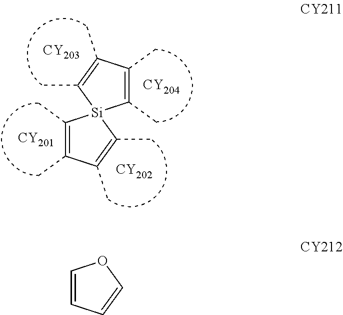

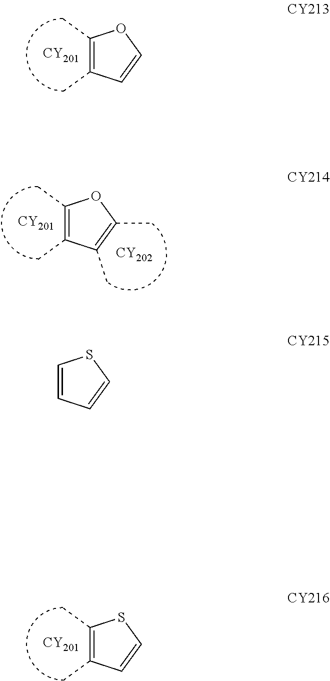

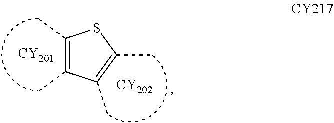

wherein, in Formulae CY201 to CY217, R_{10b} and R_{10c} may each independently the same as described with respect to R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, in Formulae CY201 to CY217, ring CY₂₀₁ to ring CY₂₀₄ may each independently be a benzene group, a naphthalene group, a phenanthrene group, or an anthracene group.

In one or more embodiments, each of Formulae 201 and 202 may include at least one selected from among groups represented by Formulae CY201 to CY203.

In one or more embodiments, Formula 201 may include at least one selected from among the groups represented by Formulae CY201 to CY203 and at least one selected from among the groups represented by Formulae CY204 to CY217.

In one or more embodiments, in Formula 201, xa1 may be 1, R₂₀₁ may be a group represented by any one selected from among Formulae CY201 to CY203, xa2 may be 0, and R₂₀₂ may be a group represented by any one selected from among Formulae CY204 to CY207.

In one or more embodiments, each of Formulae 201 and 202 may not include (e.g., may exclude any of) groups represented by Formulae CY201 to CY203.

In one or more embodiments, each of Formulae 201 and 202 may not include (e.g., may exclude any of) the groups represented by Formulae CY201 to CY203, and may include at least one selected from among the groups represented by Formulae CY204 to CY217.

In one or more embodiments, each of Formulae 201 and 202 may not include (e.g., may exclude any of) groups represented by Formulae CY201 to CY217.

In one or more embodiments, the hole transport region may include at least one of (e.g., selected from among) Compounds HT1 to HT46, 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N-(2-naphthyl)-N-phenylamino]-triphenylamine (2-TNATA), N,N′-di(naphthalen-1-yl)-N,N′-diphenyl-benzidine (NPB(NPD)), β-NPB, N,N′-bis(3-methylphenyl)-N,N′-diphenyl-[1,1′-biphenyl]-4,4′-diamine (TP D), spiro-TP D, spiro-N P B, methylated N P B, 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), 4,4′,4″-tris(N-carbazolyl)triphenylamine (TCTA), polyaniline/dodecylbenzene sulfonic acid (PANI/DBSA), poly(3,4-ethylene dioxythiophene)/poly(4-styrene sulfonate) (PEDOT/PSS), polyaniline/camphorsulfonic acid (PANI/CSA), polyaniline/poly(4-styrene sulfonate) (PANI/PSS), or any combination thereof:

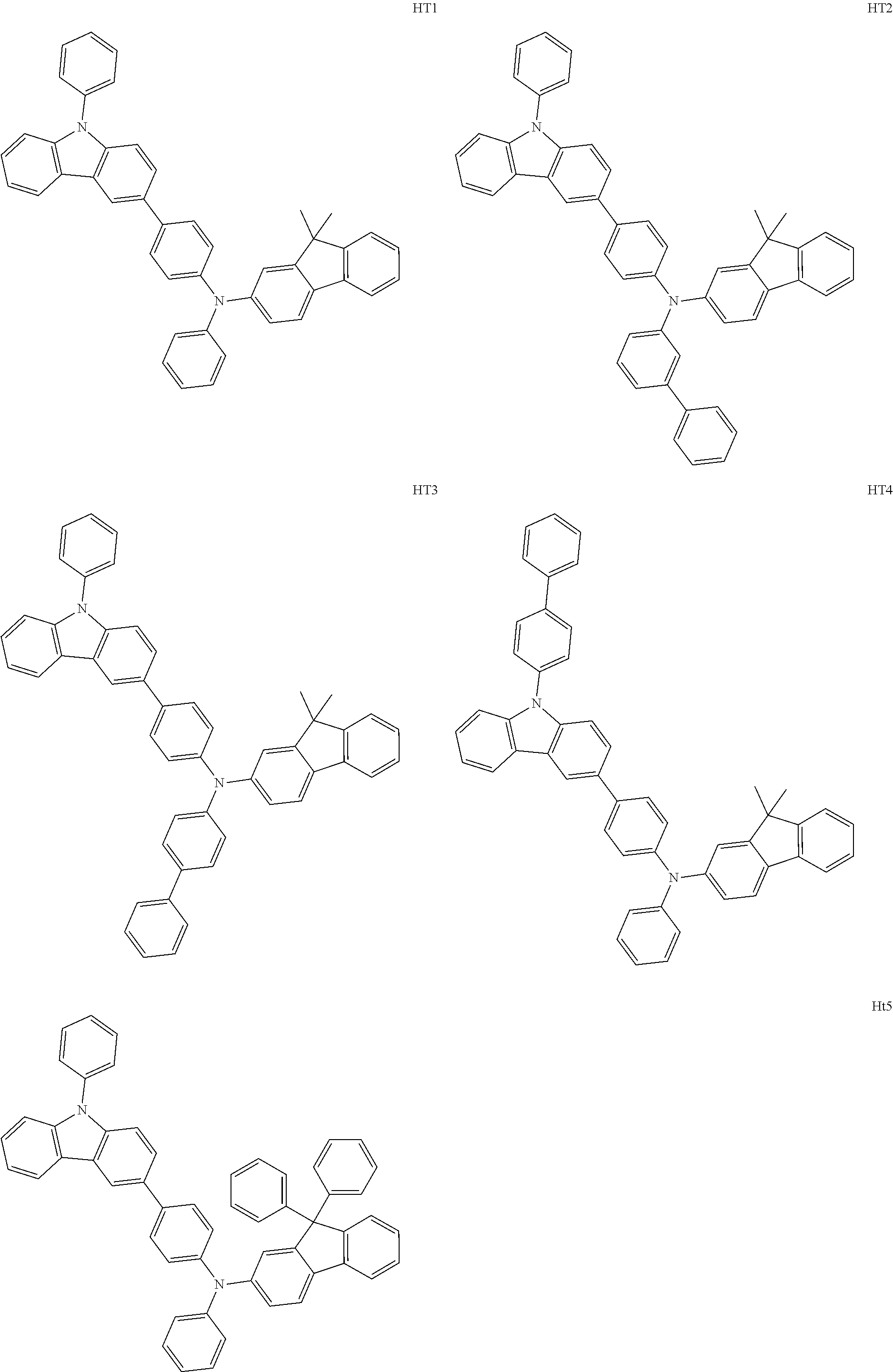 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1]

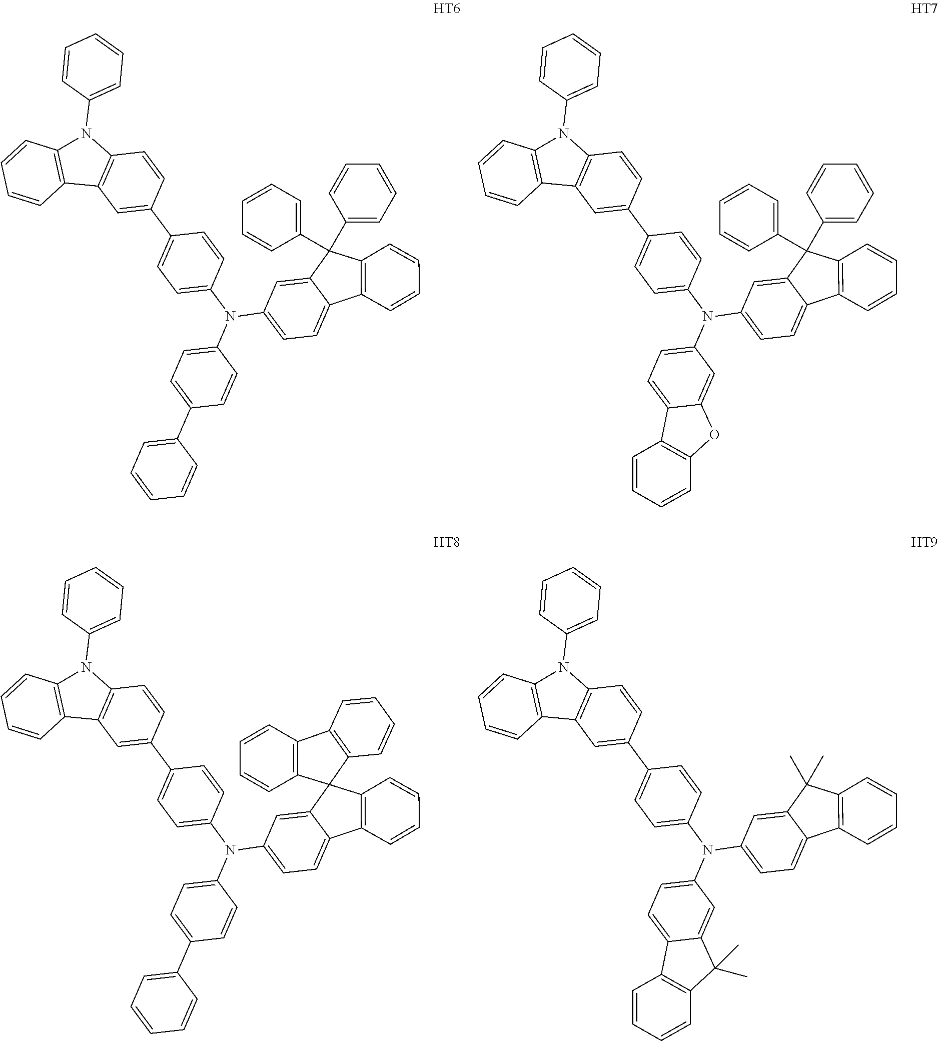 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21]

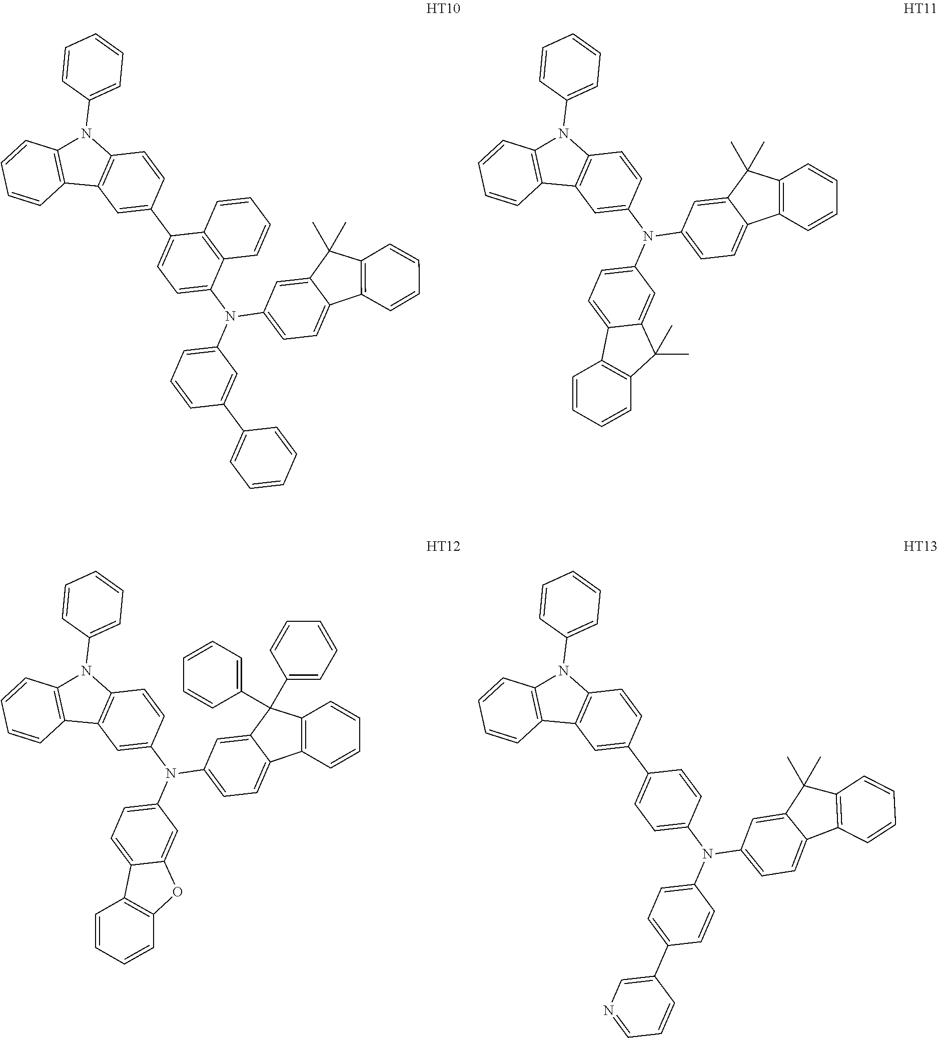 [SMILES: CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1 ; CC1(C)c2ccccc2-c2ccc(N(c3ccc(-c4cccnc4)cc3)c3ccc(-c4ccc5c(c4)c4ccccc4n5-c4ccccc4)cc3)cc21]

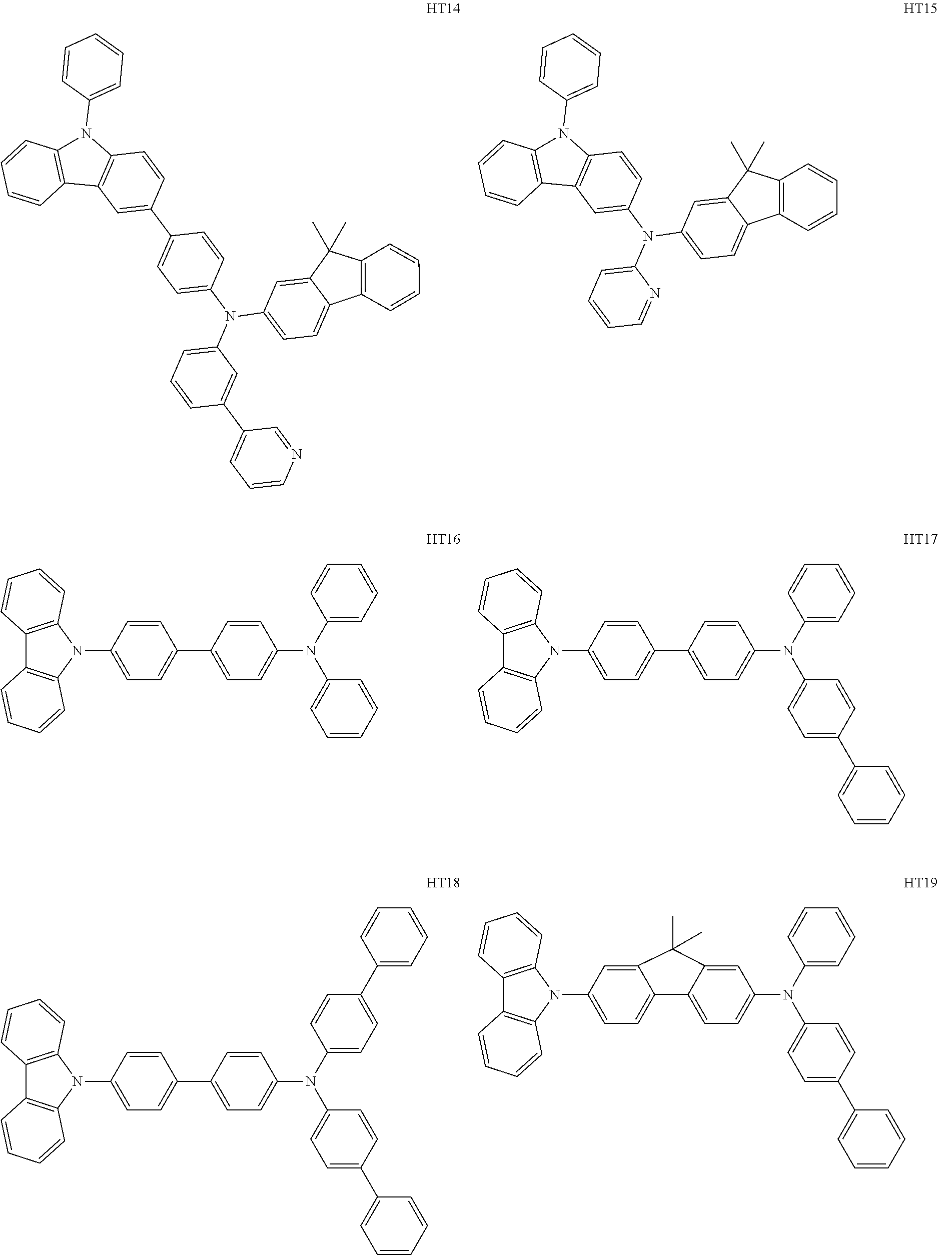 [SMILES: CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc4c(c3)c3ccccc3n4-c3ccccc3)c3ccccn3)cc21 ; c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21]

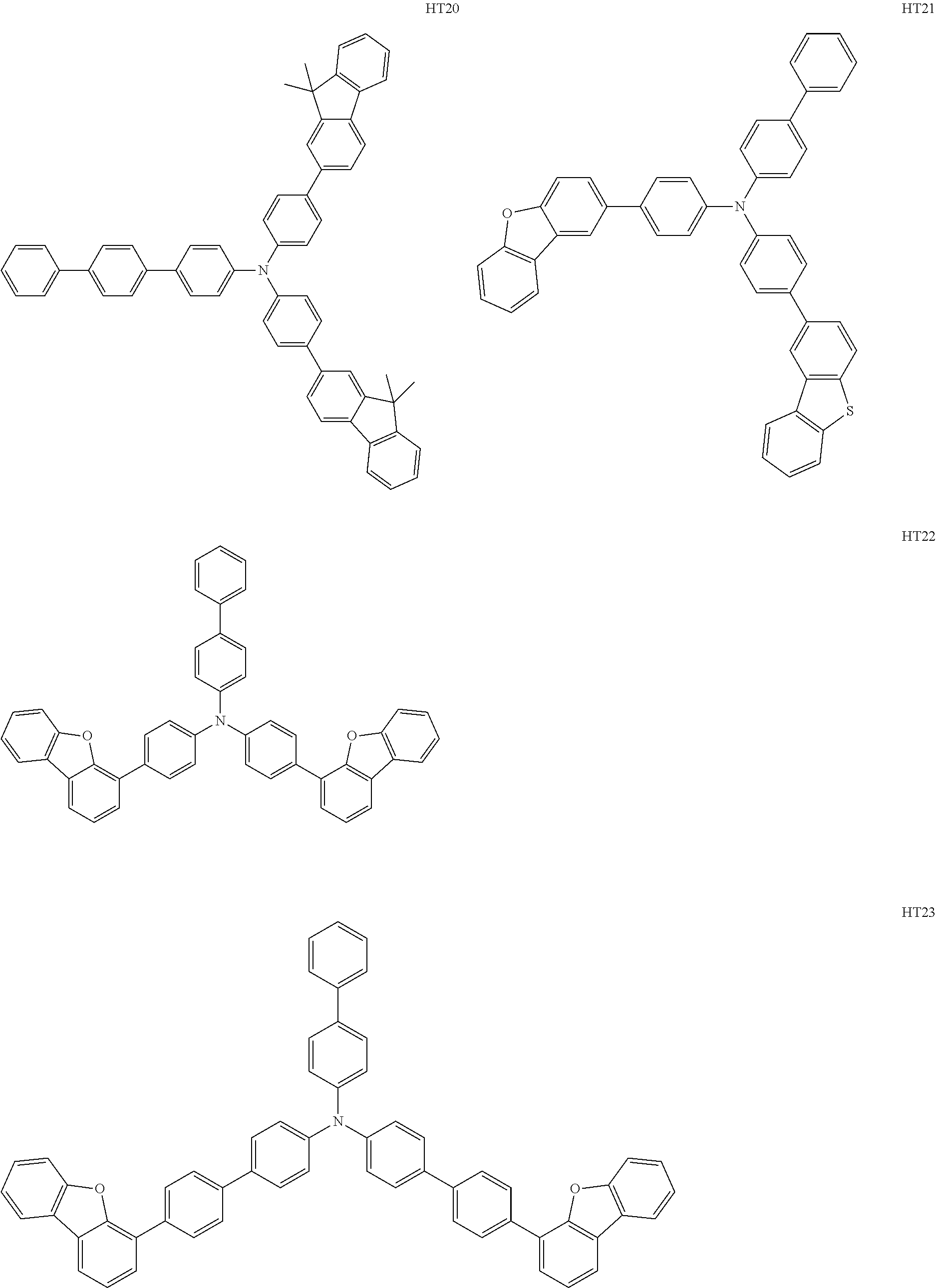 [SMILES: CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21 ; c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

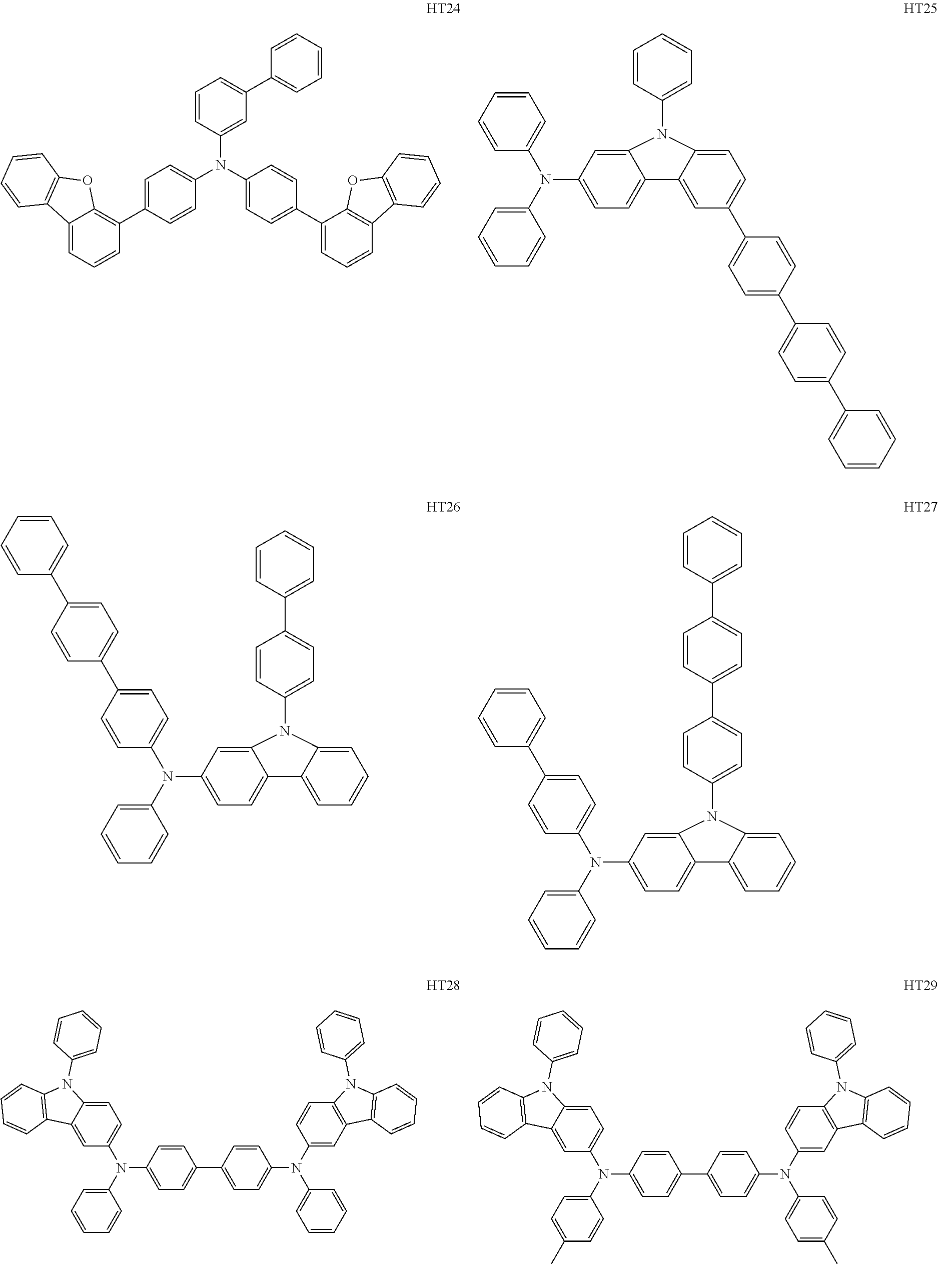

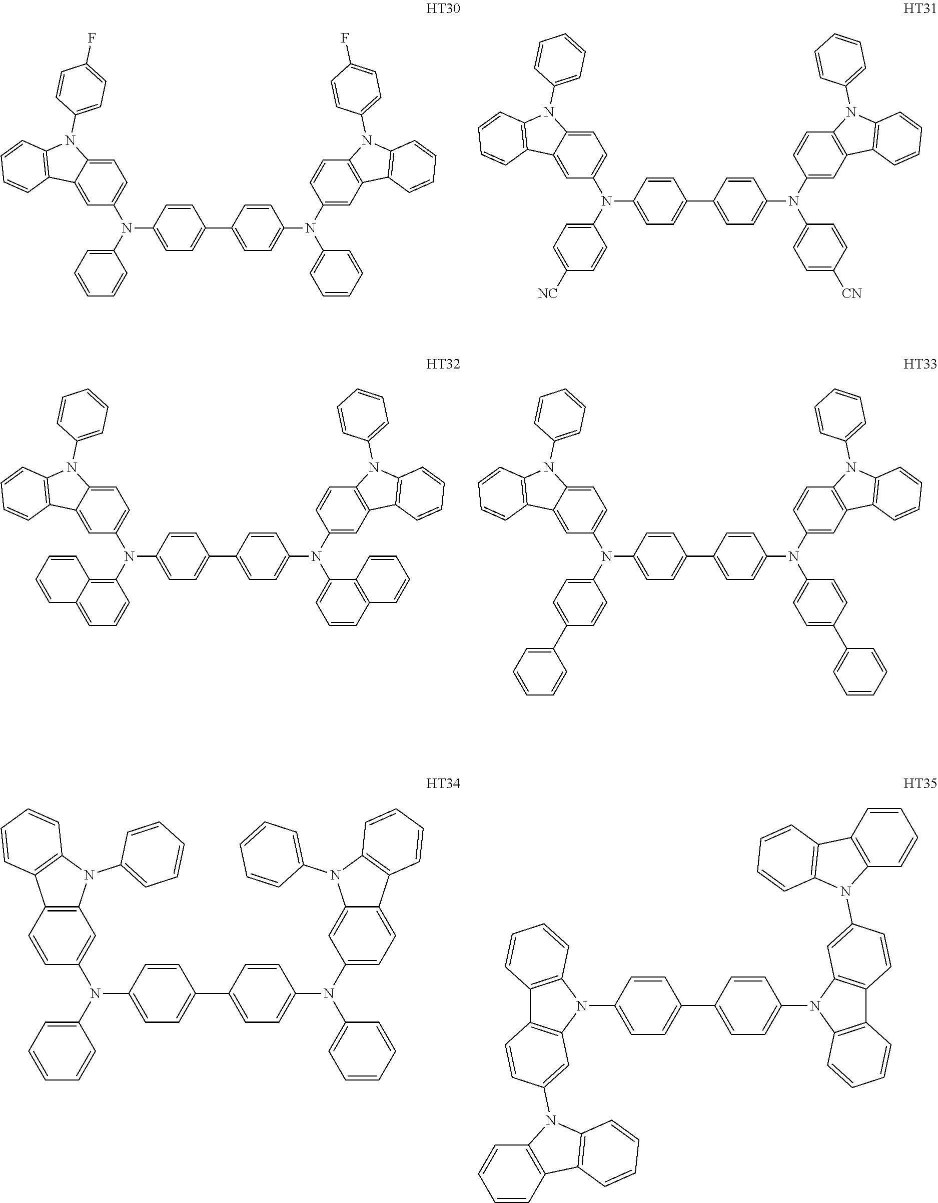

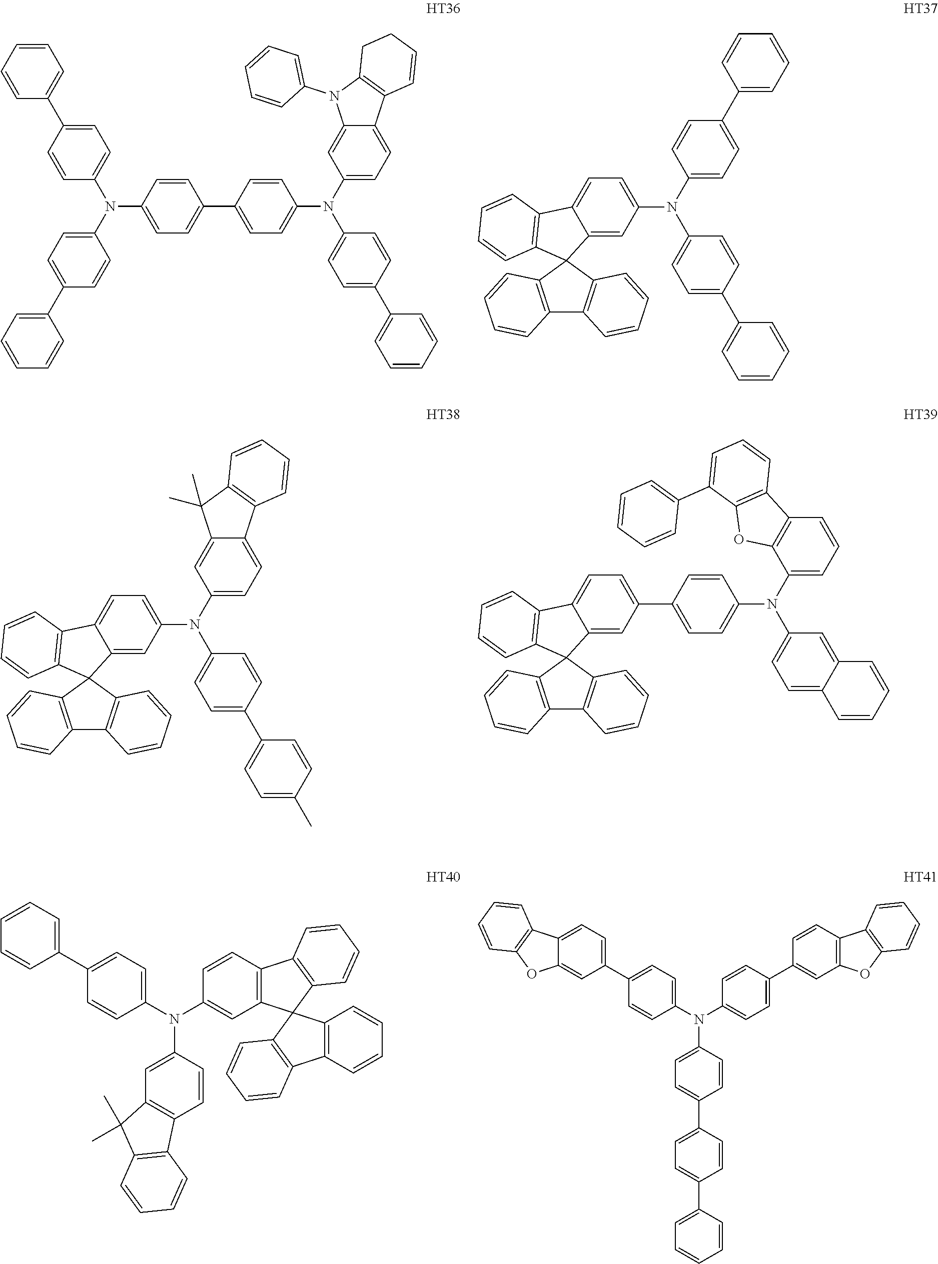

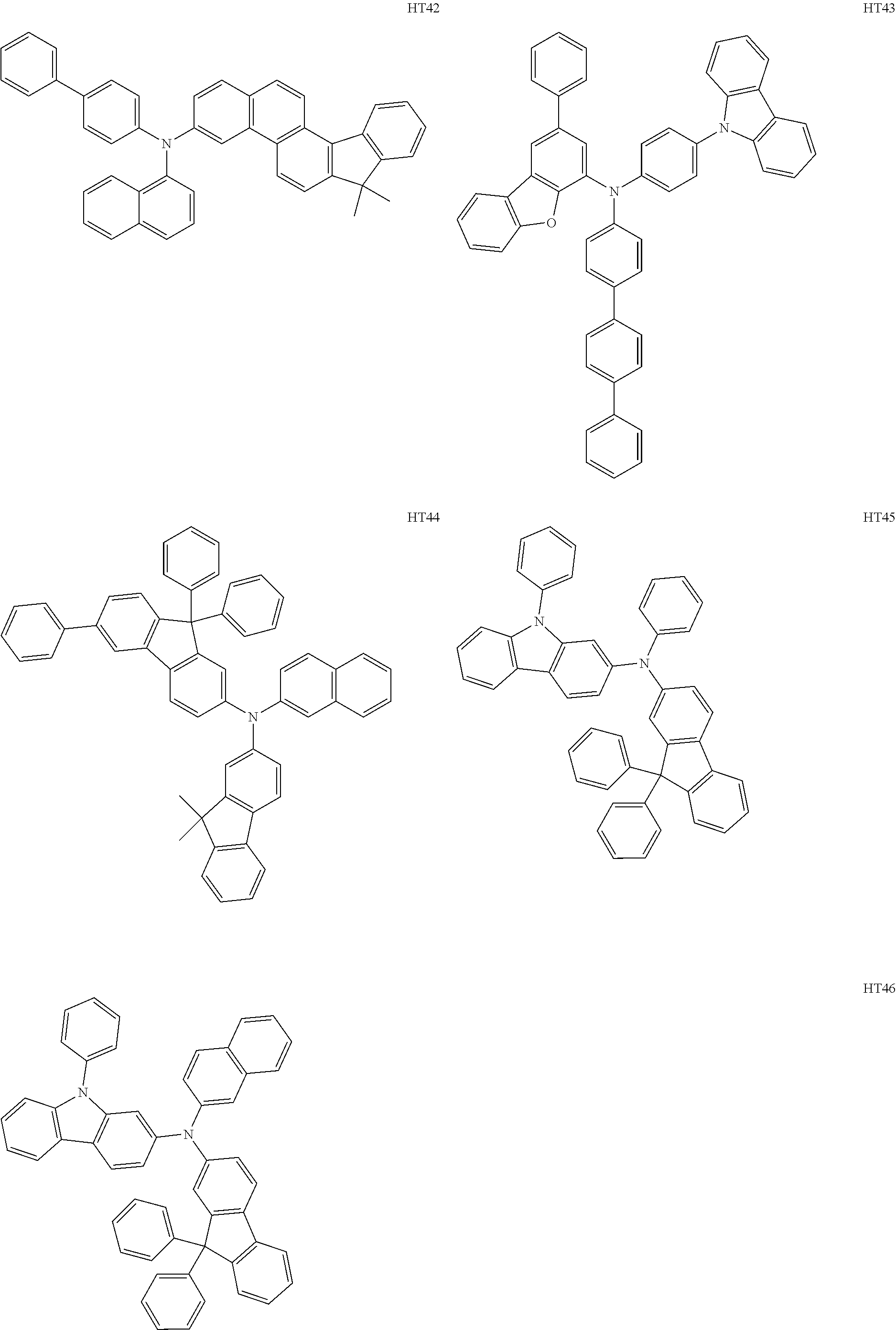 [SMILES: CC1(C)c2ccccc2-c2c1ccc1c2ccc2ccc(N(c3ccc(-c4ccccc4)cc3)c3cccc4ccccc34)cc21 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; CC1(C)c2ccccc2-c2ccc(N(c3ccc4c(c3)C(c3ccccc3)(c3ccccc3)c3ccc(-c5ccccc5)cc3-4)c3ccc4ccccc4c3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; c1ccc(-n2c3ccccc3c3ccc(N(c4ccc5c(c4)C(c4ccccc4)(c4ccccc4)c4ccccc4-5)c4ccc5ccccc5c4)cc32)cc1]

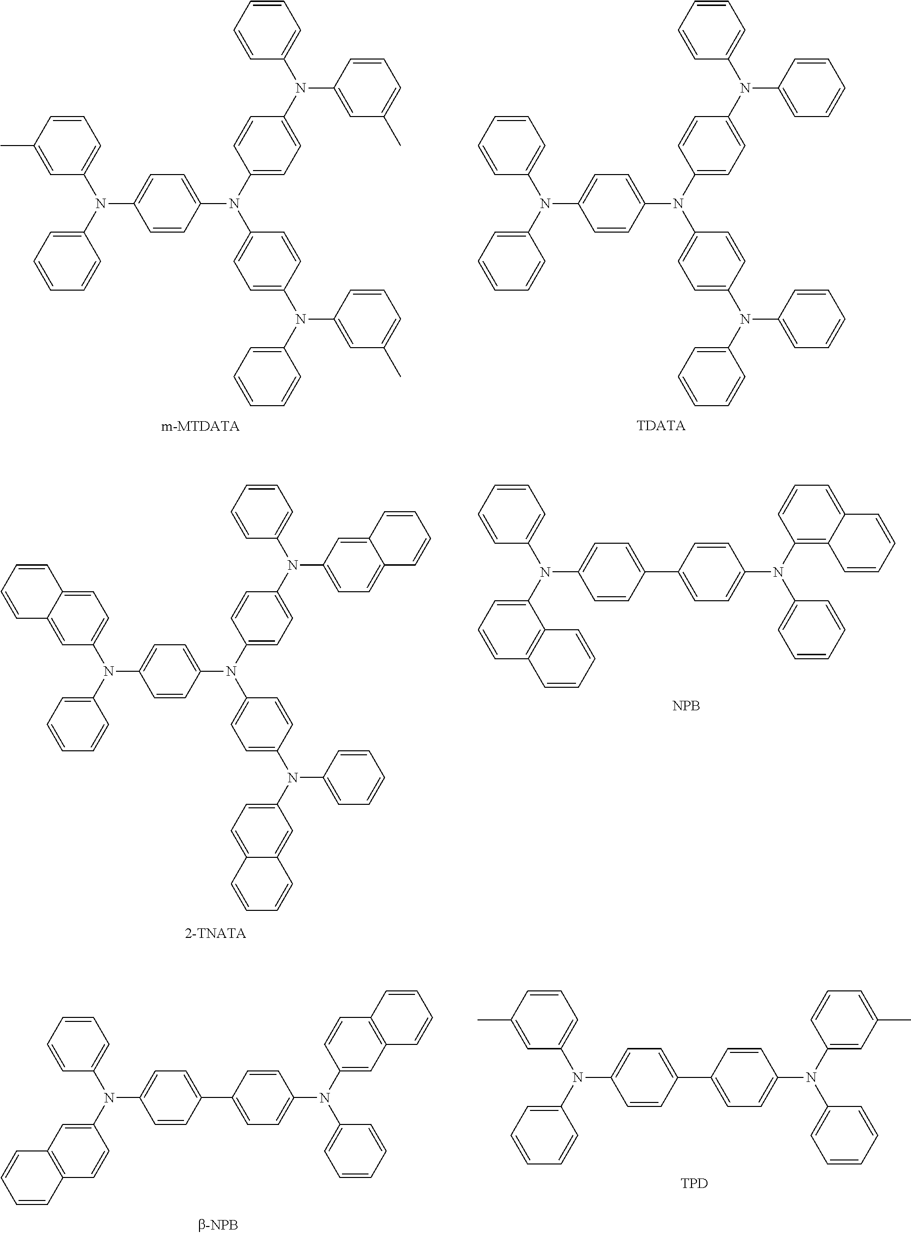

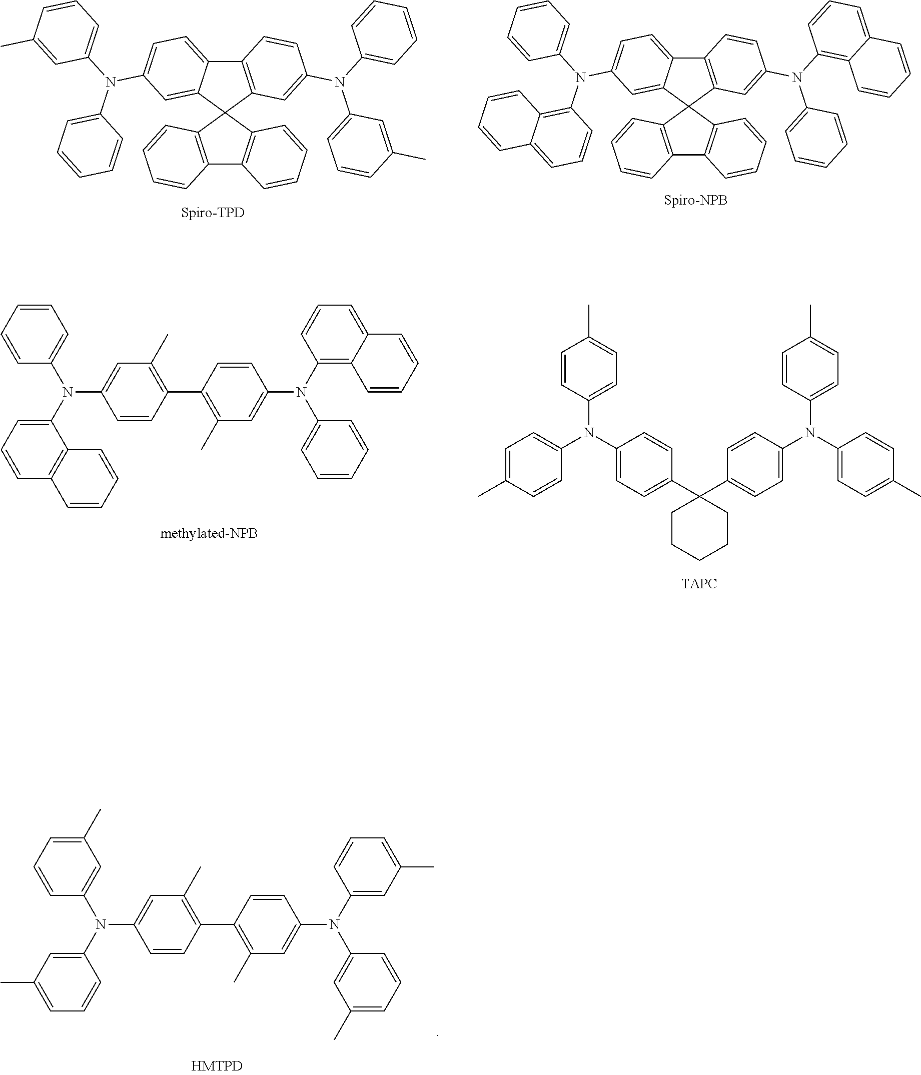

A thickness of the hole transport region may be in a range of about 50 Angstroms (Å) to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer is a layer that increases light emission efficiency by compensating for an optical resonance distance according to a wavelength of light emitted from the emission layer 130. The electron blocking layer may be a layer that prevents electron leakage from the emission layer 130 to the hole transport region 120. Materials that may be included in the hole transport region may be included in the emission auxiliary layer and the electron blocking layer.

p-Dopant

In one or more embodiments, the hole transport region 120 may further include, in addition to one or more of the aforementioned materials, a charge-generation material for the improvement of conductive properties. The charge-generation material may be uniformly (e.g., substantially uniformly) or non-uniformly dispersed in the hole transport region (e.g., in the form of a single layer including (e.g., consisting of) the charge-generation material).

The charge-generation material may be, for example, a p-dopant.

For example, the p-dopant may have a lowest unoccupied molecular orbital (LUMO) energy level of about −3.5 eV or less.

In one or more embodiments, the p-dopant may include a quinone derivative, a cyano group-containing compound, a compound including element EL1 and element EL2, or any combination thereof.

Non-limiting examples of the quinone derivative may include tetracyanoquinodimethane (TCNQ), 2,3,5,6-tetrafluoro-7,7,8,8-tetracyanoquinodimethane (F4-TCNQ), and/or the like.

Non-limiting examples of the cyano group-containing compound may include dipyrazino[2,3-f: 2′,3′-h]quinoxaline-2,3,6,7,10,11-hexacarbonitrile (HAT-CN), a compound represented by Formula 221, and/or the like:

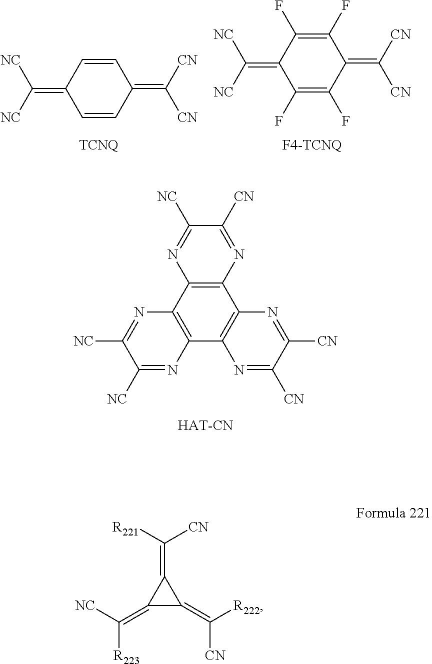

wherein, in Formula 221,
R₂₂₁ to R₂₂₃ may each independently be a C₃-C₆₀ carbocyclic group unsubstituted or substituted with at least one R₁₀ₐ or a C₁-Coo heterocyclic group unsubstituted or substituted with at least one R₁₀ₐ, and
at least one selected from among R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound including the element EL1 and the element EL2, the element EL1 may be a metal, a metalloid, or a (e.g., any suitable) combination thereof, and the element EL2 may be a non-metal, a metalloid, or a (e.g., any suitable) combination thereof.

Non-limiting examples of the metal may include: alkali metals (e.g., lithium (Li), sodium (Na), potassium (K), rubidium (Rb), cesium (Cs), and/or the like); alkaline earth metals (e.g., beryllium (Be), magnesium (Mg), calcium (Ca), strontium (Sr), barium (Ba), and/or the like); transition metals (e.g., titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), and/or the like); post-transition metals (e.g., zinc (Zn), indium (In), tin (S n), and/or the like); lanthanide metals (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and/or the like); and/or the like.

Non-limiting examples of the metalloid may include silicon (Si), antimony (S b), tellurium (Te), and/or the like.

Non-limiting examples of the non-metal may include oxygen (O), a halogen (e.g., F, Cl, Br, I, and/or the like), and/or the like.

For example, the compound including element EL1 and element EL2 may include metal oxides, metal halides (e.g., metal fluorides, metal chlorides, metal bromides, metal iodides, and/or the like), metalloid halides (e.g., metalloid fluorides, metalloid chlorides, metalloid bromides, metalloid iodides, and/or the like), metal tellurides, or any combination thereof.

Non-limiting examples of the metal oxide may include a tungsten oxide (for example, WO, W₂O₃, WO₂, WO₃, W₂O₅, and/or the like), a vanadium oxide (for example, VO, V₂O₃, VO₂, V₂O₅, and/or the like), a molybdenum oxide (for example, MoO, Mo₂O₃, MoO₂, MoO₃, Mo₂O₅, and/or the like), a rhenium oxide (for example, ReO₃, and/or the like), and/or the like.

Non-limiting examples of the metal halide may include alkali metal halides, alkaline earth metal halides, transition metal halides, post-transition metal halides, lanthanide metal halides, and/or the like.

Non-limiting examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, CsI, and/or the like.

Non-limiting examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, Sr₁₂, BaI₂, and/or the like.

Non-limiting examples of the transition metal halide may include titanium halides (e.g., TiF₄, TiCl₄, TiBr₄, TiI₄, and/or the like), zirconium halides (e.g., ZrF₄, ZrCl₄, ZrBr₄, ZrI₄, and/or the like), hafnium halides (e.g., HfF₄, HfCl₄, HfBr₄, HfI₄, and/or the like), vanadium halides (e.g., VF₃, VCl₃, VBr₃, VI₃, and/or the like), niobium halides (e.g., NbF₃, NbCl₃, NbBr₃, NbI₃, and/or the like), tantalum halides (e.g., TaF₃, TaCl₃, TaBr₃, TaI₃, and/or the like), chromium halides (e.g., CrF₃, CrCl₃, CrBr₃, CrI₃, and/or the like), molybdenum halides (e.g., MoF₃, MOCl₃, MoBr₃, MoI₃, and/or the like), tungsten halides (e.g., WF₃, WCl₃, WBr₃, WI₃, and/or the like), manganese halides (e.g., MnF₂, MnCl₂, MnBr₂, MnI₂, and/or the like), technetium halides (e.g., TcF₂, TcCl₂, TcBr₂, TcI₂, and/or the like), rhenium halides (e.g., ReF₂, ReCl₂, ReBr₂, ReI₂, and/or the like), iron(II) halides (e.g., FeF₂, FeCl₂, FeBr₂, FeI₂, and/or the like), ruthenium halides (e.g., RuF₂, RuCl₂, RuBr₂, RuI₂, and/or the like), osmium halides (e.g., OsF₂, OsCl₂, OsBr₂, OsI₂, and/or the like), cobalt halides (e.g., CoF₂, CoCl₂, CoBr₂, CoI₂, and/or the like), rhodium halides (e.g., RhF₂, RhCl₂, RhBr₂, RhI₂, and/or the like), iridium halides (e.g., IrF₂, IrCl₂, IrBr₂, IrI₂, and/or the like), nickel halides (e.g., NiF₂, NiCl₂, NiBr₂, NiI₂, and/or the like), palladium halides (e.g., PdF₂, PdCl₂, PdBr₂, PdI₂, and/or the like), platinum halides (e.g., PtF₂, PtCl₂, PtBr₂, PtI₂, and/or the like), copper(I) halides (e.g., CuF, CuCl, CuBr, CuI, and/or the like), silver halides (e.g., AgF, AgCl, AgBr, AgI, and/or the like), gold halides (e.g., AuF, AuCl, AuBr, AuI, and/or the like), and/or the like.

Non-limiting examples of the post-transition metal halide may include a zinc halide (for example, ZnF₂, ZnCl₂, ZnBr₂, ZnI₂, and/or the like), an indium halide (for example, InI₃, and/or the like), a tin halide (for example, SnI₂, and/or the like), and/or the like.

Non-limiting examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and/or the like.

Non-limiting examples of the metalloid halide may include an antimony halide (e.g., SbCl₅, and/or the like) and/or the like.

Non-limiting examples of the metal telluride may include alkali metal tellurides (e.g., Li₂Te, Na₂Te, K₂Te, Rb₂Te, Cs₂Te, and/or the like), alkaline earth metal tellurides (e.g., BeTe, MgTe, CaTe, SrTe, BaTe, and/or the like), transition metal tellurides (e.g., TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and/or the like), post-transition metal tellurides (e.g., ZnTe, and/or the like), lanthanide metal tellurides (e.g., LaTe, CeTe, PrTe, NdTe, PmTe, EuTe, GdTe, TbTe, DyTe, HoTe, ErTe, TmTe, YbTe, LuTe, and/or the like), and/or the like.

### Emission Layer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure in which two or more layers selected from among a red emission layer, a green emission layer, and a blue emission layer contact each other or are separated from each other to emit white light (e.g., combined white light). In one or more embodiments, the emission layer may have a structure in which two or more materials selected from among a red light-emitting material, a green light-emitting material, and a blue light-emitting material are mixed with each other in a single layer to emit white light (e.g., combined white light).

In one or more embodiments, the emission layer may include a host and a dopant. The dopant may include a phosphorescent dopant, a fluorescent dopant, or any combination thereof.

An amount of the dopant in the emission layer may be in a range of about 0.01 parts by weight to about 15 parts by weight, based on 100 parts by weight of the host.

In one or more embodiments, the emission layer may include quantum dots.

In one or more embodiments, the emission layer may include a delayed fluorescence material. The delayed fluorescence material may serve as a host or a dopant in the emission layer.

A thickness of the emission layer may be in a range of about 100 Å to about 1,000 Å, for example, about 200 Å to about 600 Å. When the thickness of the emission layer is within these ranges, excellent or suitable luminescence characteristics may be obtained without a substantial increase in driving voltage.

### Host

In one or more embodiments, the host may include a compound represented by Formula 301:

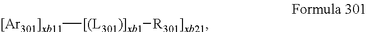

wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each independently the same as described with respect to Q₁.

For example, in one or more embodiments, if (e.g., when) xb11 in Formula 301 is 2 or more, two or more Ar₃₀₁(s) may be linked to each other via a single bond.

In one or more embodiments, the host may include a compound represented by Formula 301-1, a compound represented by Formula 301-2, or any combination thereof:

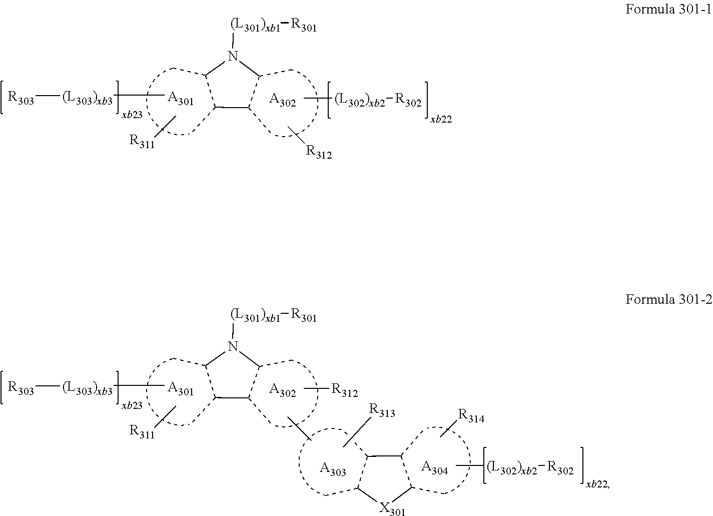

wherein, in Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ are each as described elsewhere herein,
L₃₀₂ to L₃₀₄ may each independently be the same as described with respect to L₃₀₁,
xb2 to xb4 may each independently be the same as described with respect to xb1, and
R₃₀₂ to R₃₀₅ and R₃₁₁ to R₃₁₄ may each independently be the same as described with respect to R₃₀₁.

In one or more embodiments, the host may include an alkaline earth metal complex, a post-transition metal complex, or any combination thereof. In one or more embodiments, the host may include a Be complex (e.g., Compound H55), an Mg complex, a Zn complex, or any combination thereof.

In one or more embodiments, the host may include: at least one of (e.g., selected from among) Compounds H1 to H128; 9,10-di(2-naphthyl)anthracene (ADN); 2-methyl-9,10-bis(naphthalen-2-yl)anthracene (MADN); 9,10-di-(2-naphthyl)-2-t-butyl-anthracene (TBADN); 4,4′-bis(N-carbazolyl)-1,1′-biphenyl (CBP); 1,3-di(carbazol-9-yl)benzene (mCP); 1,3,5-tri(carbazol-9-yl)benzene (TCP); or any combination thereof:

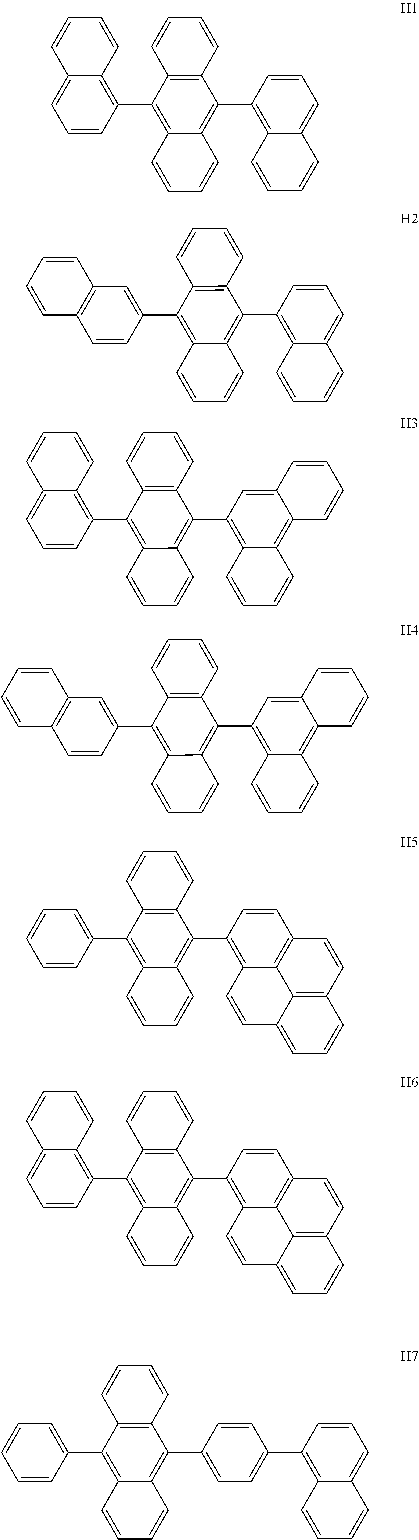

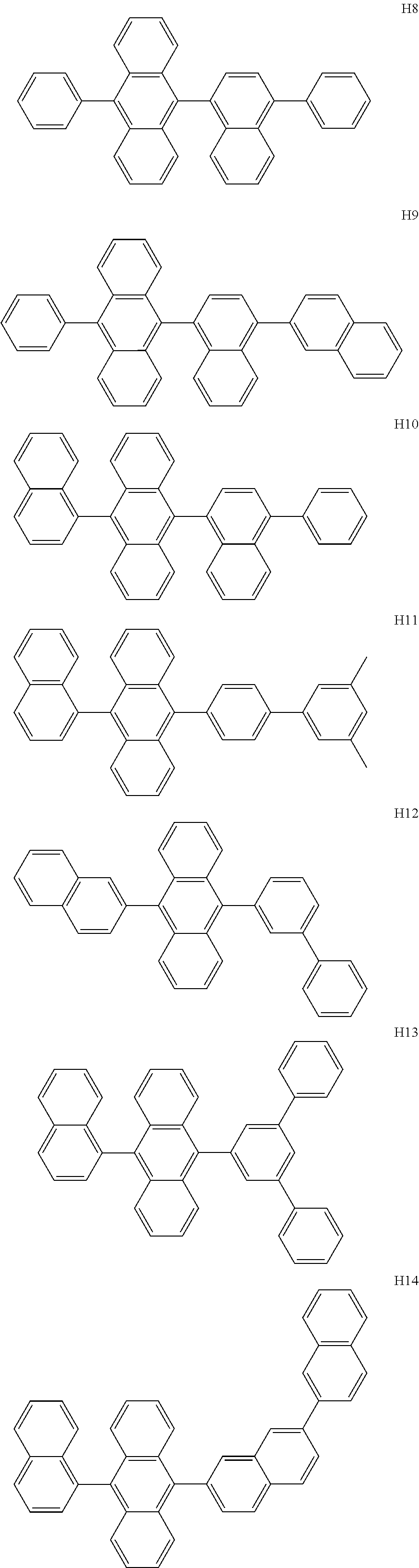

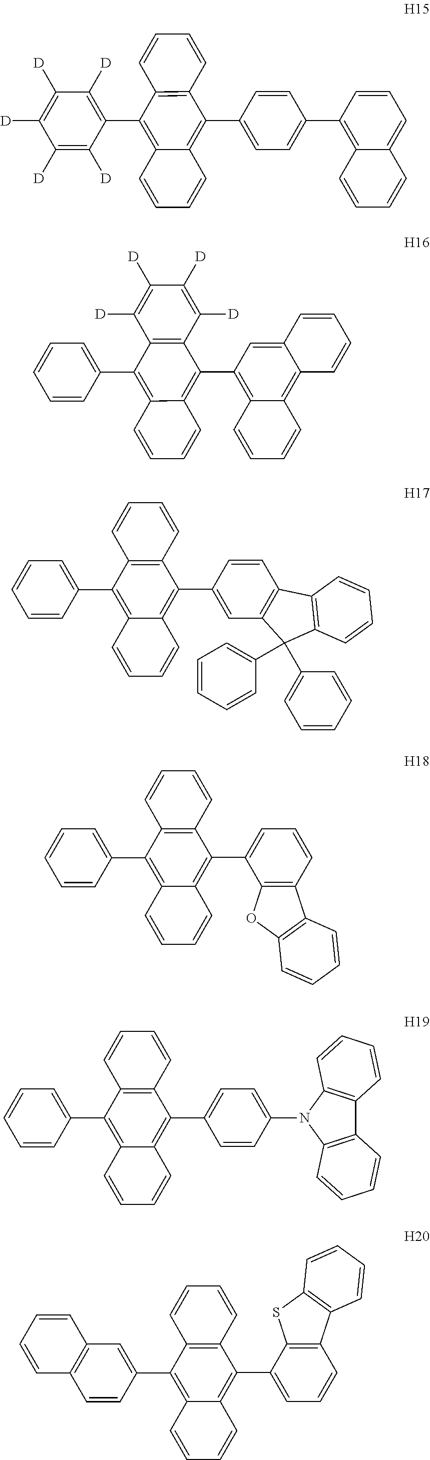

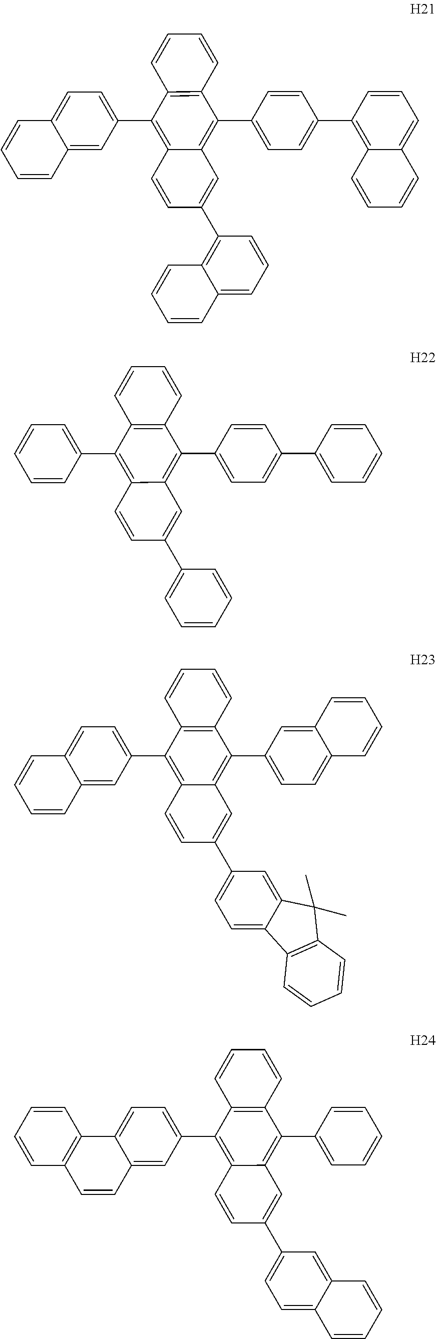

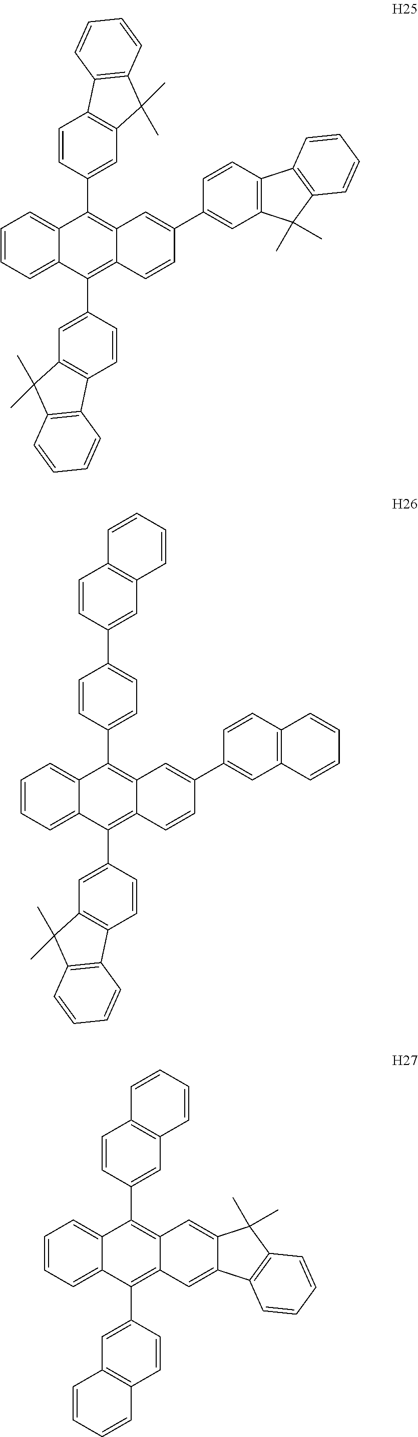

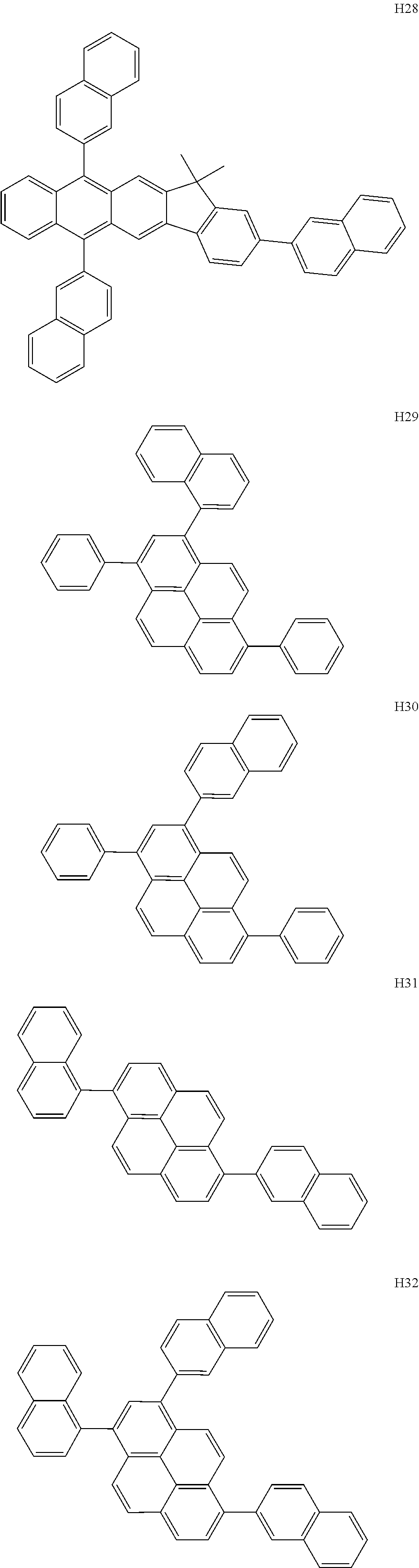

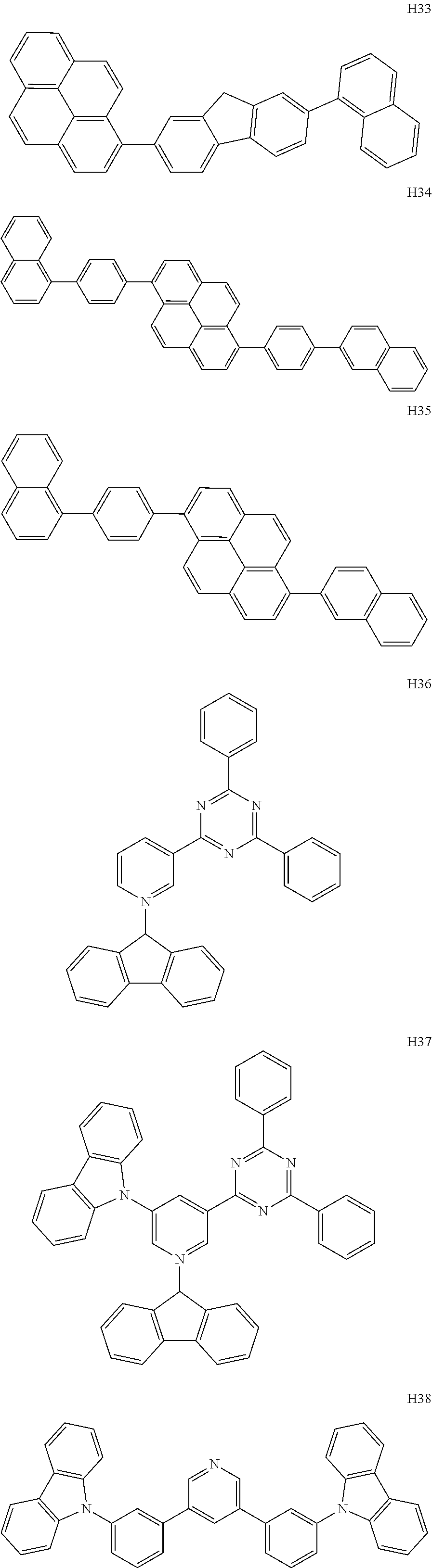

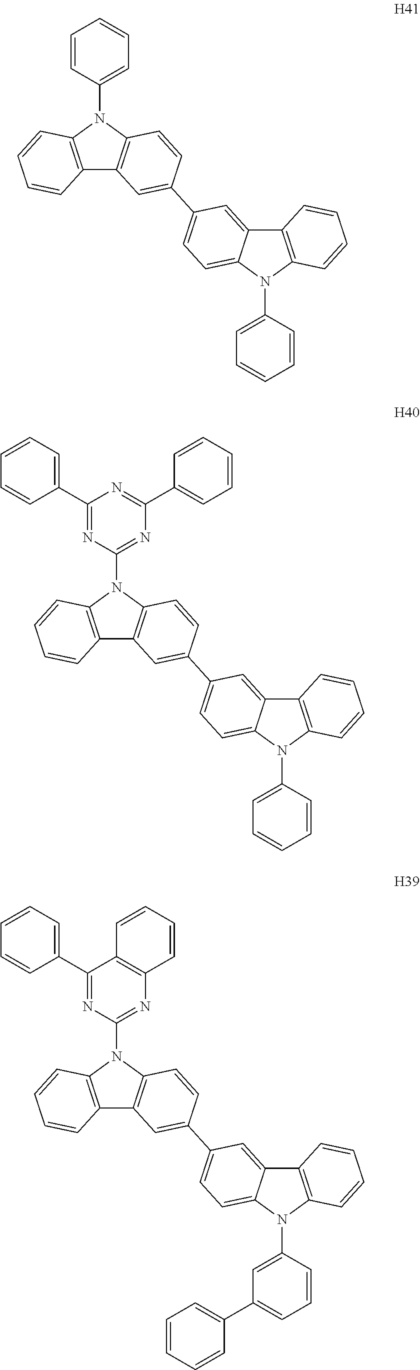 [SMILES: c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1]

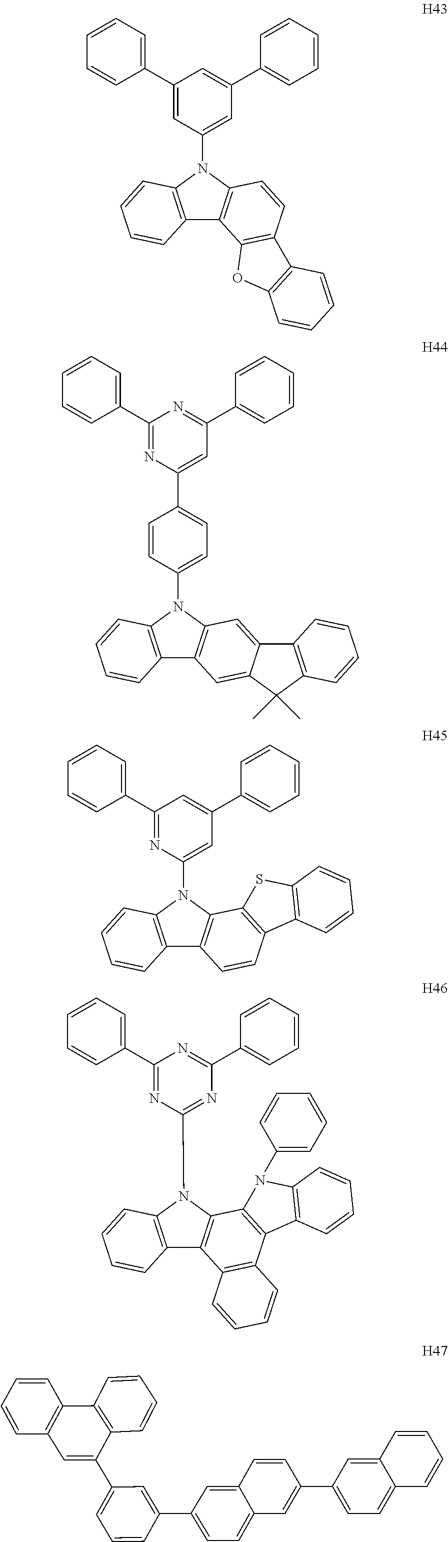

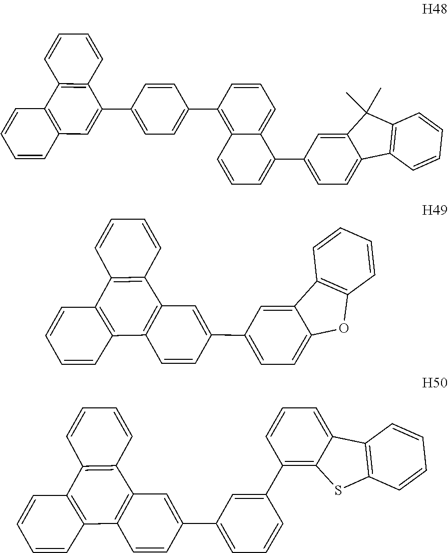

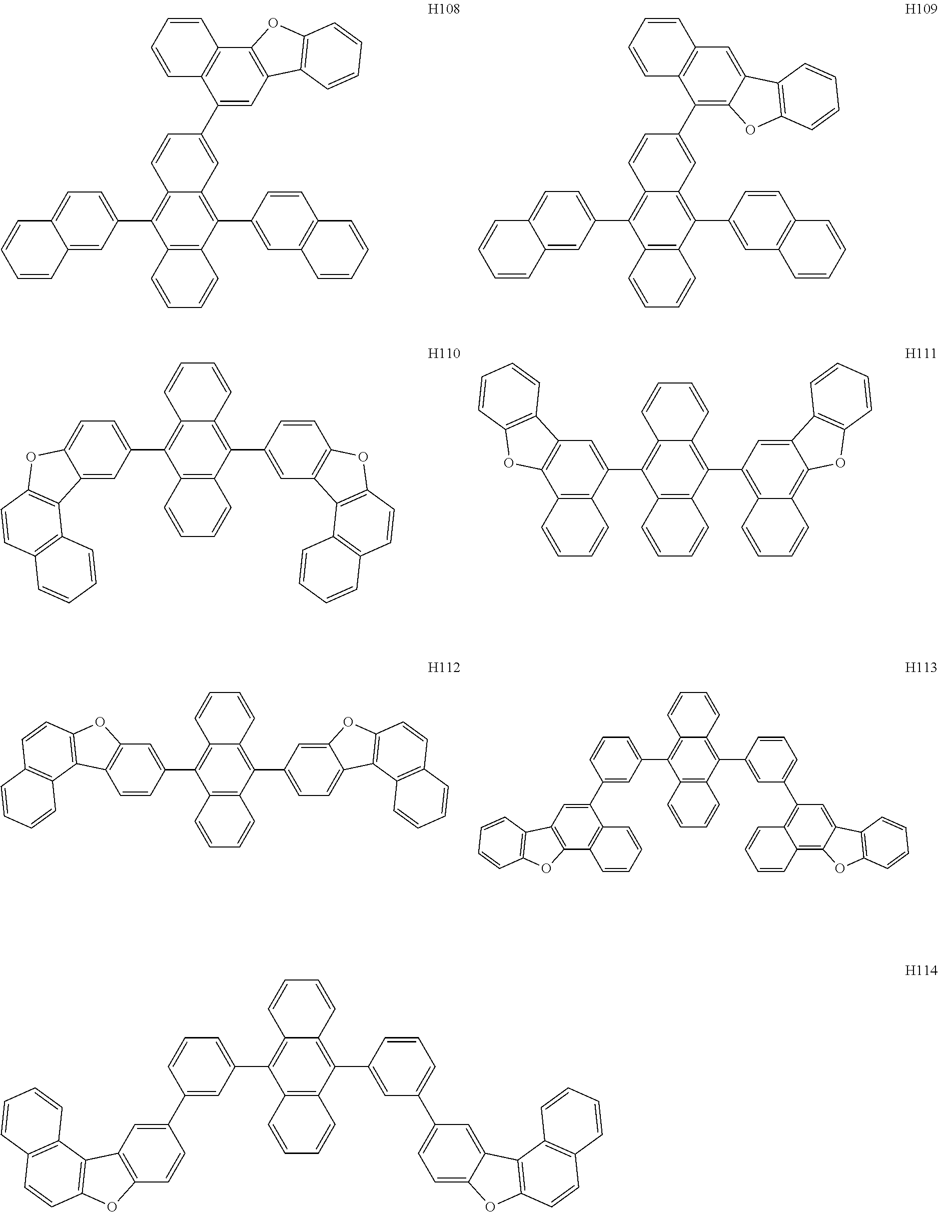

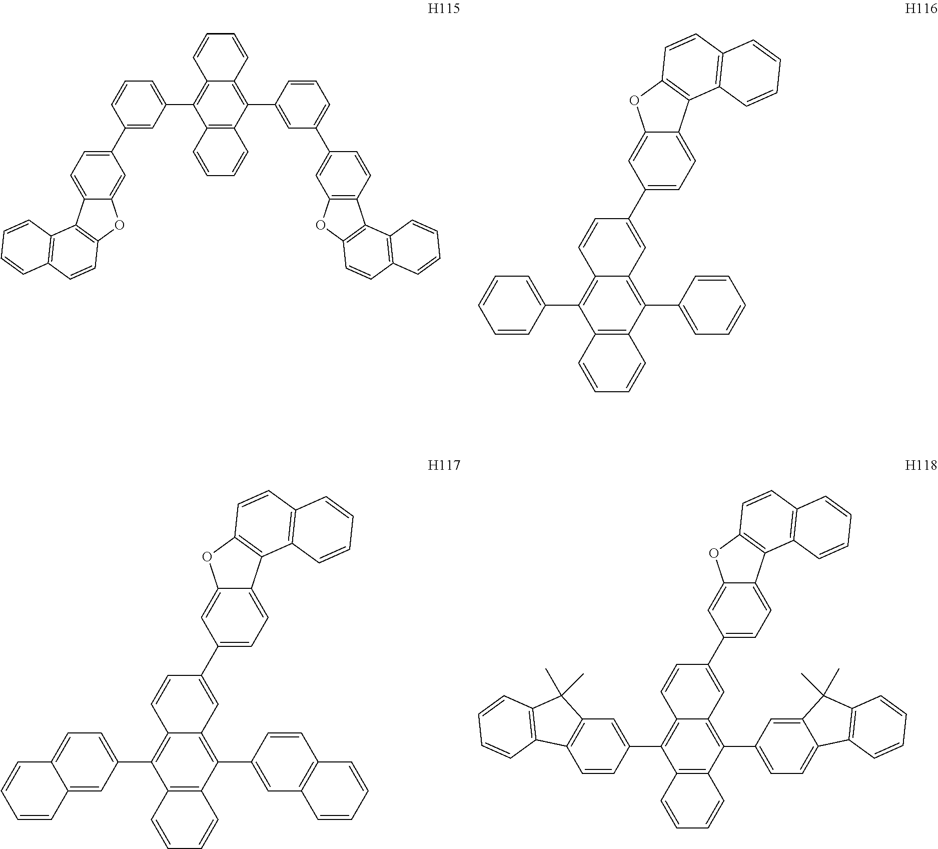 [SMILES: c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

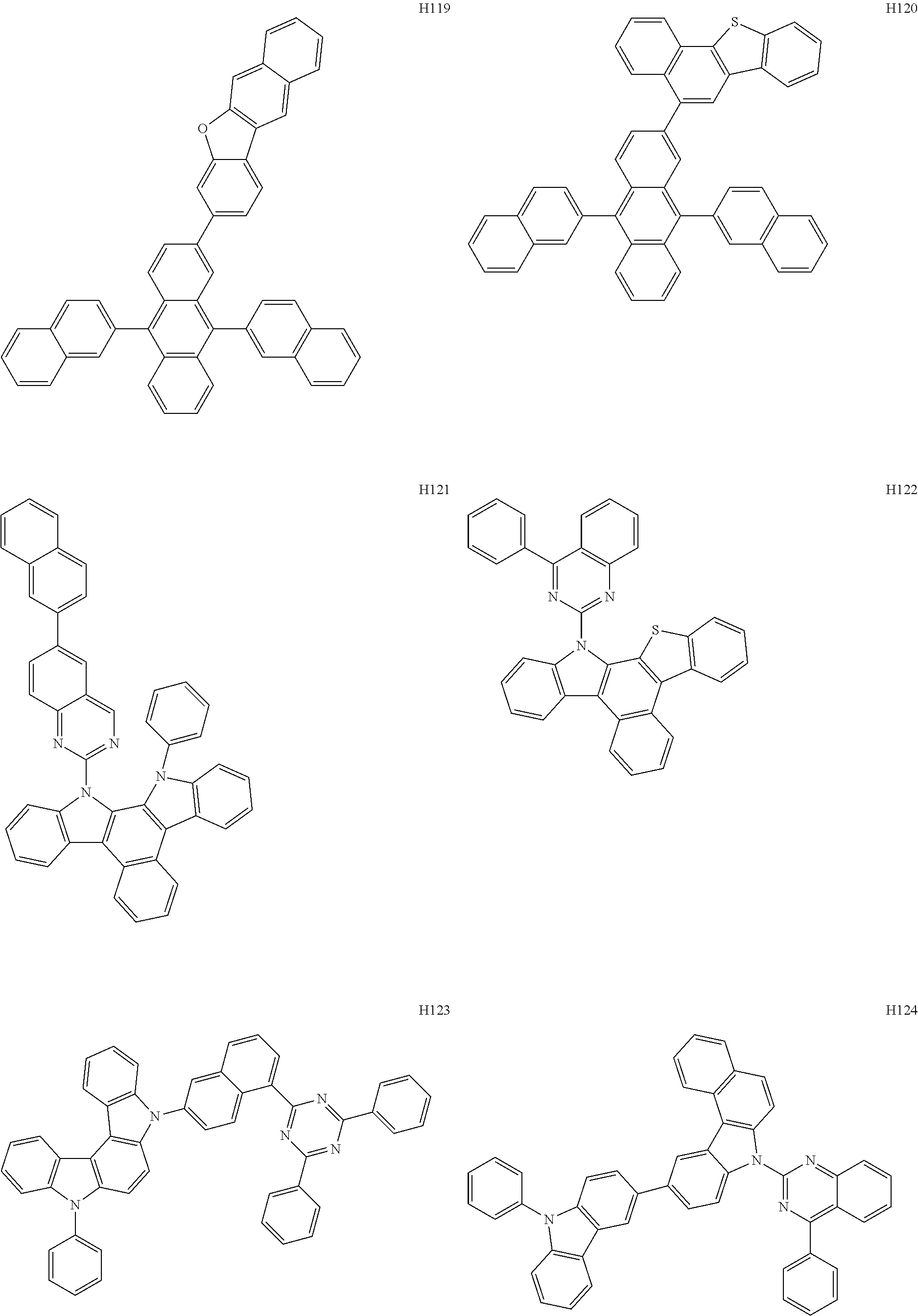

### Phosphorescent Dopant

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

In one or more embodiments, the phosphorescent dopant may include an organometallic compound represented by Formula 401:

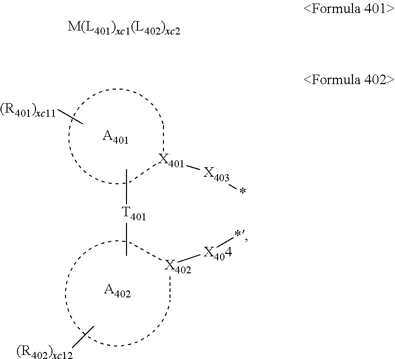

wherein, in Formulae 401 and 402,
M may be a transition metal (e.g., iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein, if (e.g., when) xc1 is 2 or more, two or more L₄₀₁(s) may be identical to or different from each other,
L₄₀₂ may be an organic ligand, and xc2 may be 0, 1, 2, 3, or 4, wherein, if (e.g., when) xc2 is 2 or more, two or more L₄₀₂(s) may be identical to or different from each other,
X₄₀₁ and X₄₀₂ may each independently be nitrogen or carbon, ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)=*′, or *═C═*′,
X₄₀₃ and X₄₀₄ may each independently be a chemical bond (e.g., a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁ to Q₄₁₄ may each independently be the same as described with respect to Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each independently be the same as described with respect to Q₁,
xc11 and xc12 may each independently be an integer from 0 to 10, and
* and *′ in Formula 402 each indicate a binding site to M in Formula 401.

For example, in one or more embodiments, in Formula 402, i) X₄₀₁ may be nitrogen and X₄₀₂ may be carbon, or ii) each of X₄₀₁ and X₄₀₂ may be nitrogen.

In one or more embodiments, if (e.g., when) xc1 in Formula 401 is 2 or more, two ring A₄₀₁(s) among two or more L₄₀₁(s) may optionally be linked to each other via T₄₀₂, which is a linking group, and/or two ring A₄₀₂(s) among two or more L₄₀₁(s) may optionally be linked to each other via T₄₀₃, which is a linking group (see Compounds PD1 to PD4 and PD7). T₄₀₂ and T₄₀₃ are each the same as described with respect to T₄₀₁.

In Formula 401, L₄₀₂ may be an organic ligand. For example, L₄₀₂ may include a halogen, a diketone group (e.g., an acetylacetonate group), a carboxylic acid group (e.g., a picolinate group), —C(═O), an isonitrile group, a —CN group, a phosphorus-containing group (e.g., a phosphine group, a phosphite group, and/or the like), or any combination thereof.

The phosphorescent dopant may include, for example, one of (e.g., at least one selected from among) Compounds PD1 to PD39, or any combination thereof:

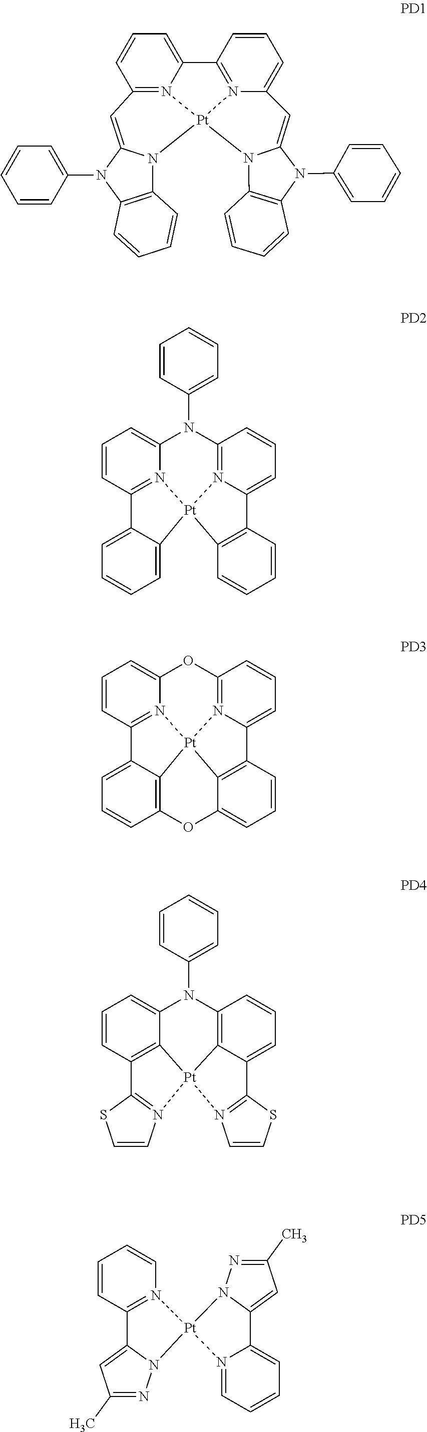

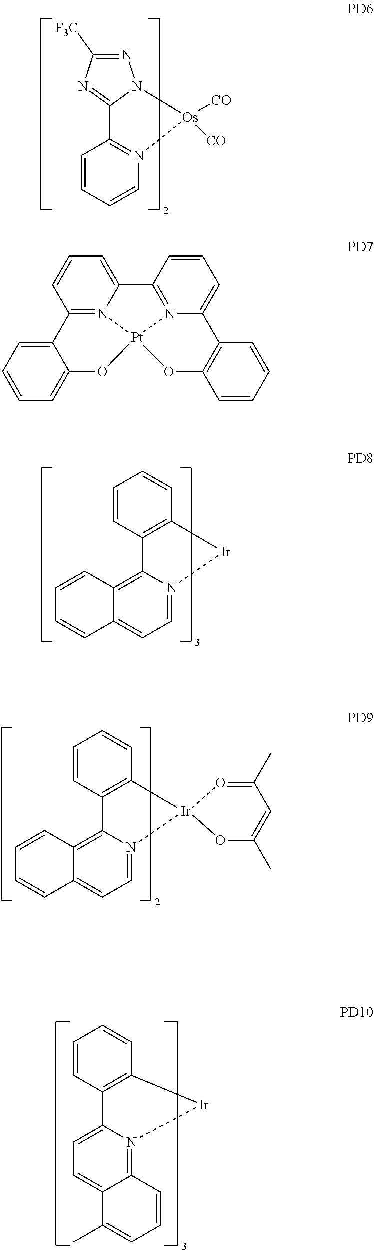

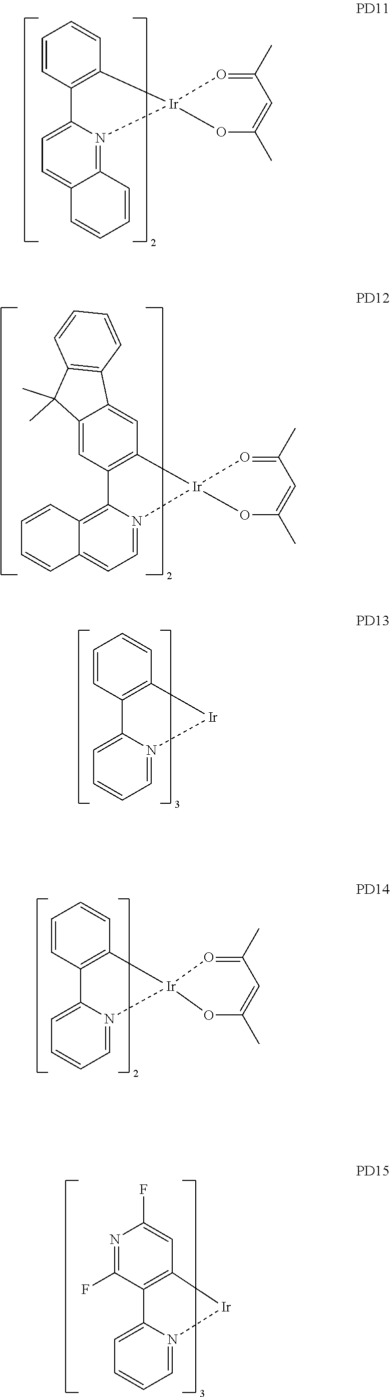

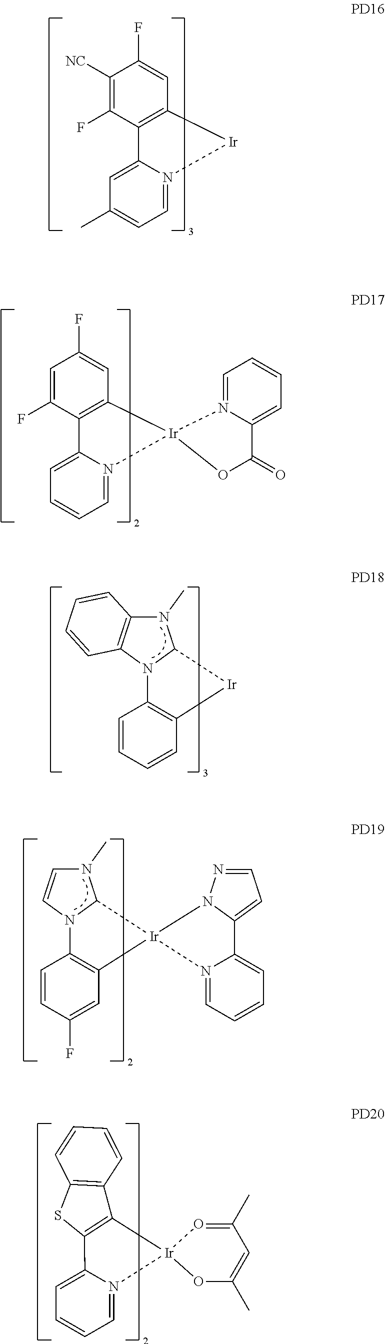

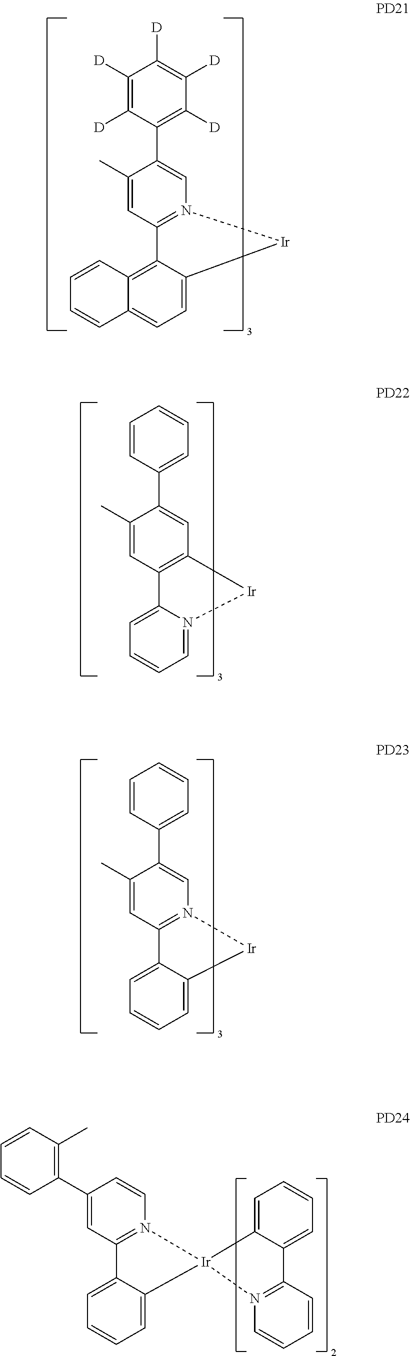

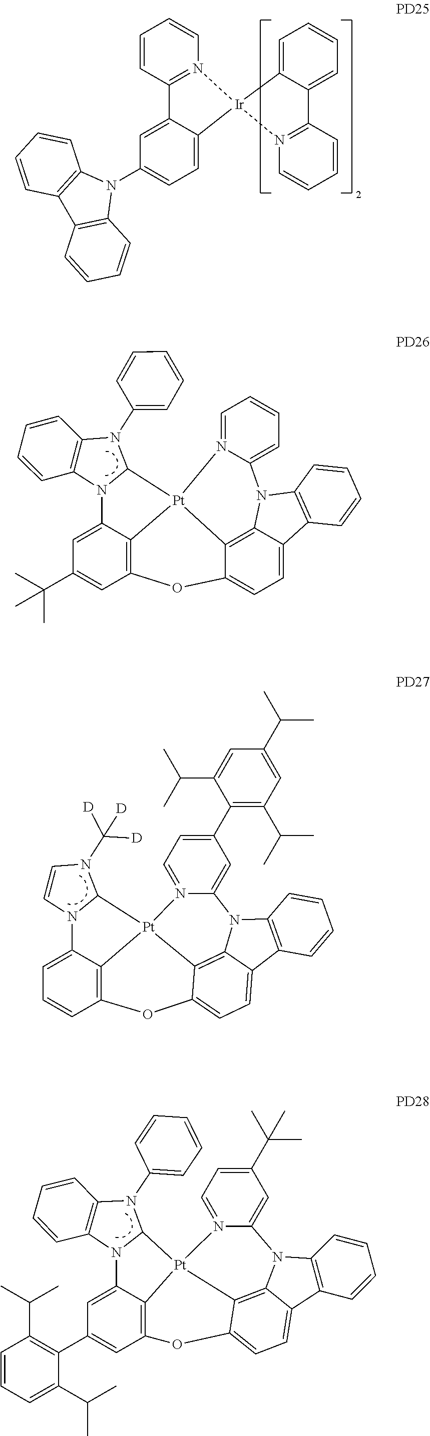

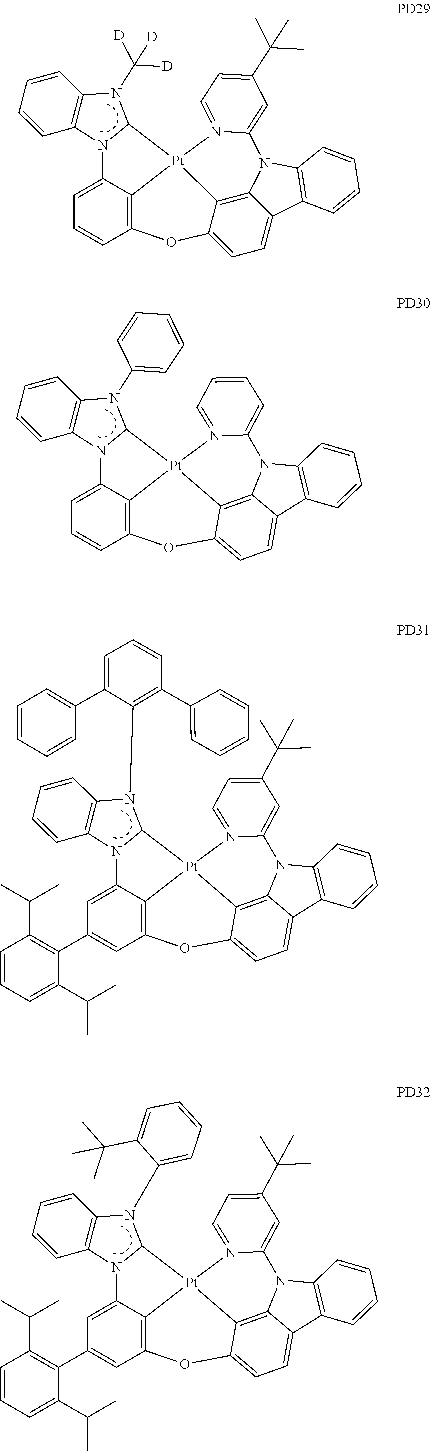

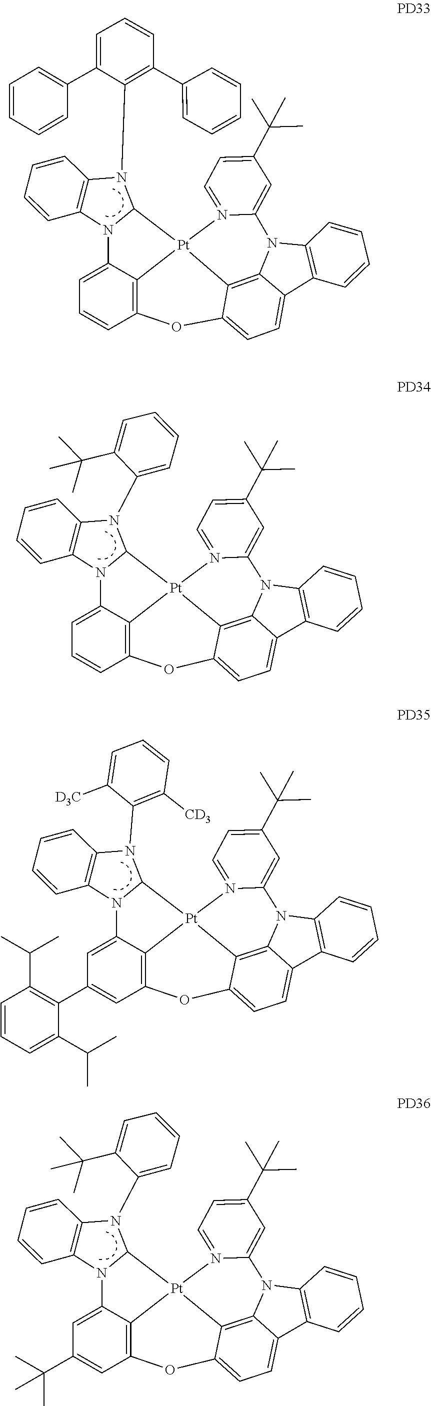

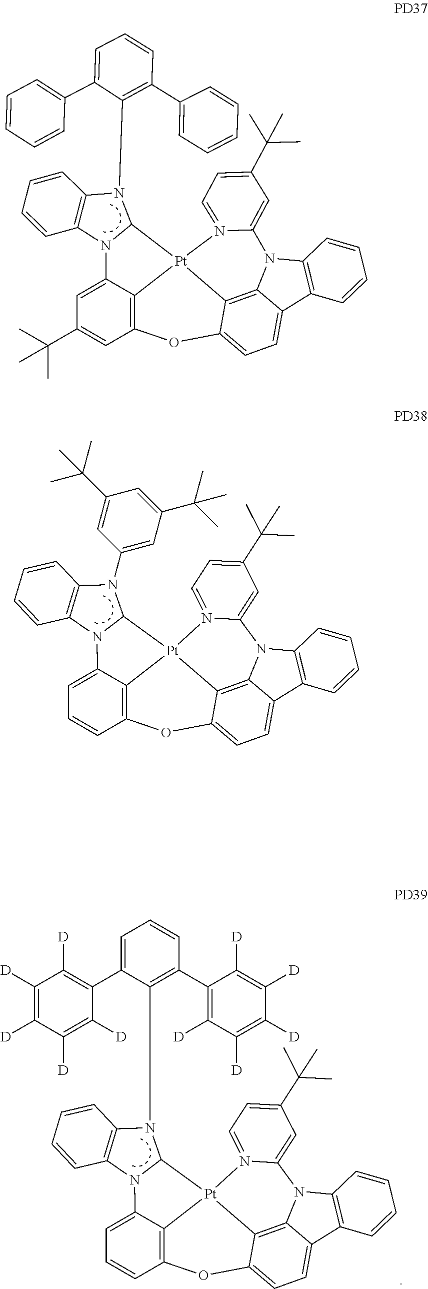

### Fluorescent Dopant

The fluorescent dopant may include an amine group-containing compound, a styryl group-containing compound, or any combination thereof.

For example, in one or more embodiments, the fluorescent dopant may include a compound represented by Formula 501:

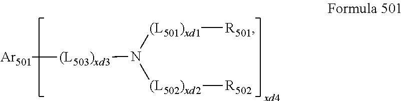

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

For example, in one or more embodiments, Ar₅₀₁ in Formula 501 may be a condensed cyclic group (e.g., an anthracene group, a chrysene group, a pyrene group, and/or the like) in which three or more monocyclic groups are condensed with each other.

For example, in one or more embodiments, xd4 in Formula 501 may be 2.

For example, in one or more embodiments, the fluorescent dopant may include: at least one of (e.g., selected from among) Compounds FD1 to FD37; 4,4′-bis(2,2-diphenylvinyl)-1,1′-biphenyl (DPVBi); 4,4′-bis[4-(N,N-diphenylamino)styryl]biphenyl (DPAVBi); or any combination thereof:

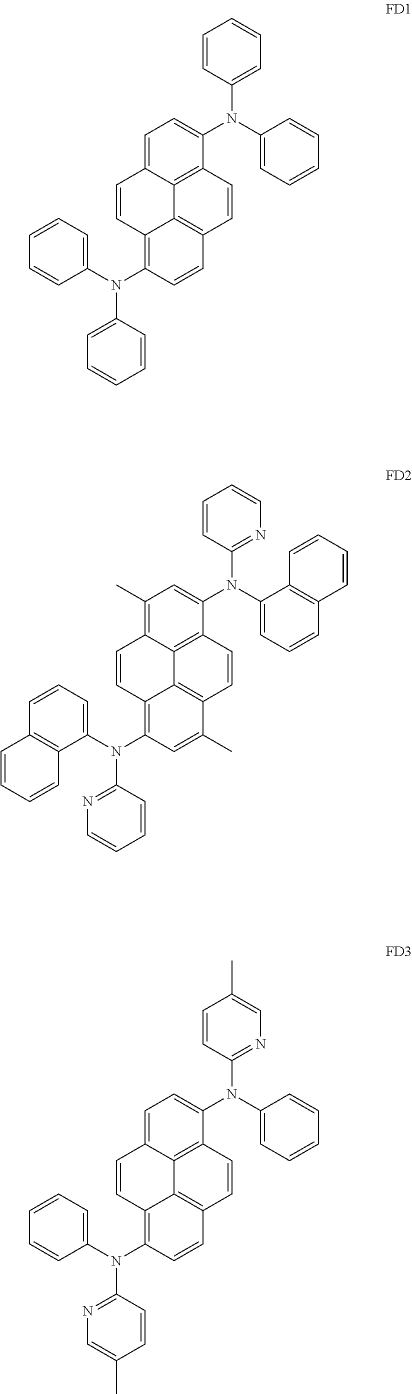 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1]

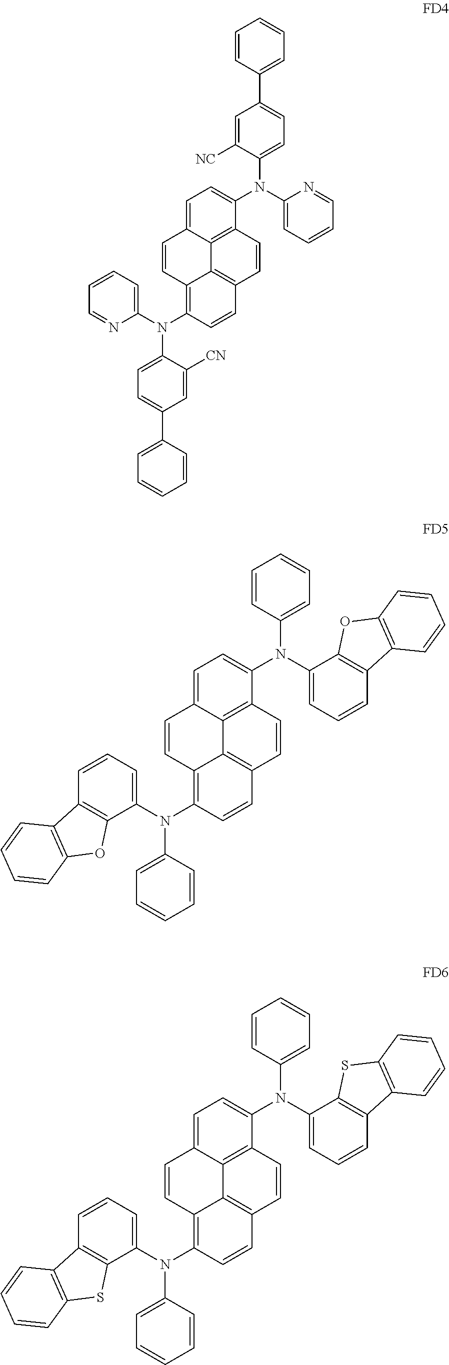 [SMILES: N#Cc1cc(-c2ccccc2)ccc1N(c1ccccn1)c1ccc2ccc3c(N(c4ccccn4)c4ccc(-c5ccccc5)cc4C#N)ccc4ccc1c2c43 ; c1ccc(N(c2ccc3ccc4c(N(c5ccccc5)c5cccc6c5oc5ccccc56)ccc5ccc2c3c54)c2cccc3c2oc2ccccc23)cc1 ; c1ccc(N(c2ccc3ccc4c(N(c5ccccc5)c5cccc6c5sc5ccccc56)ccc5ccc2c3c54)c2cccc3c2sc2ccccc23)cc1]

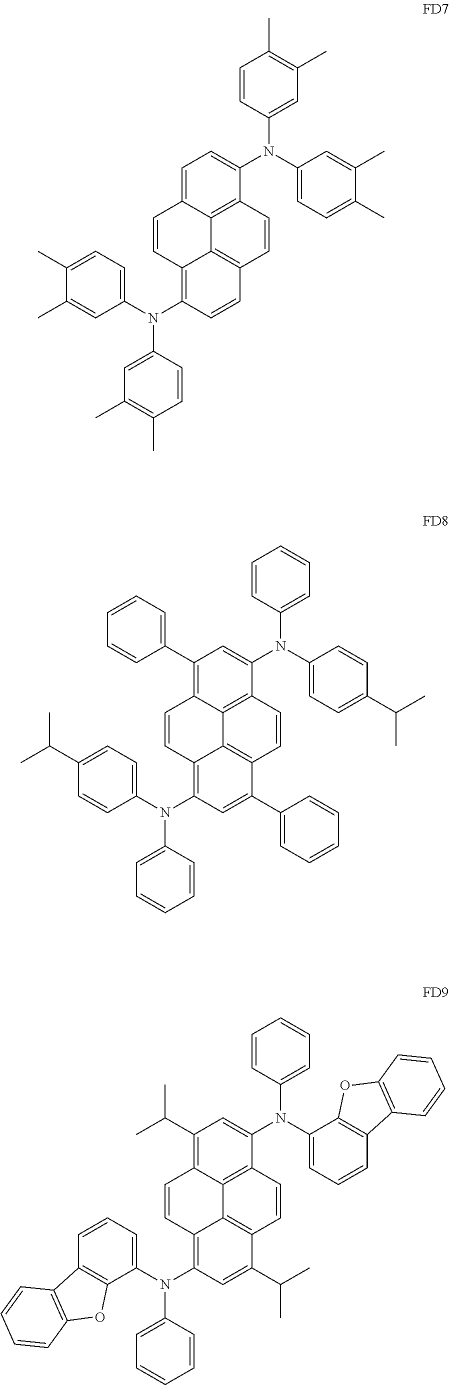

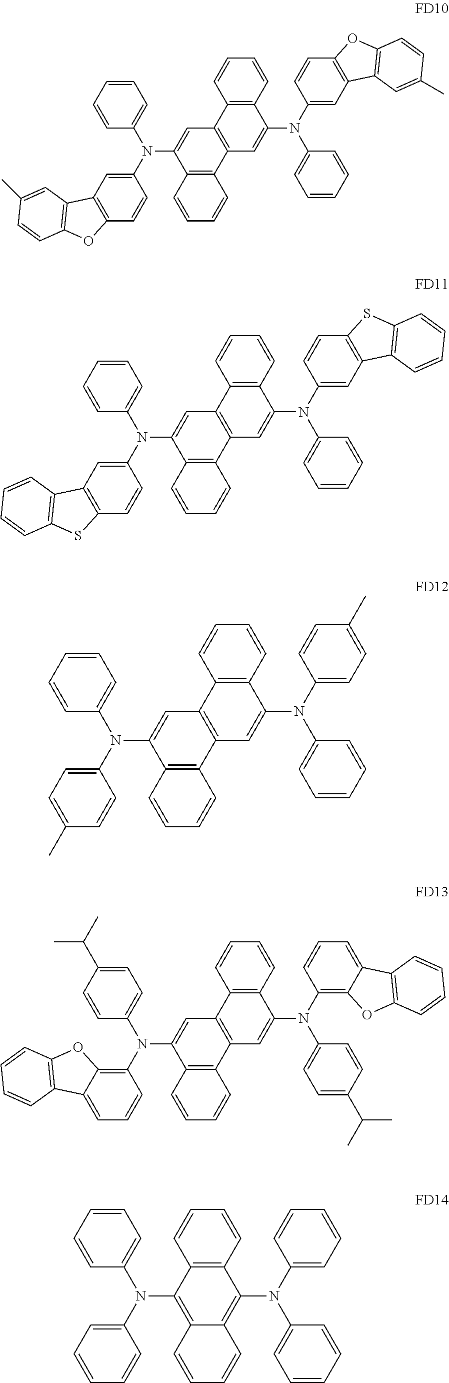

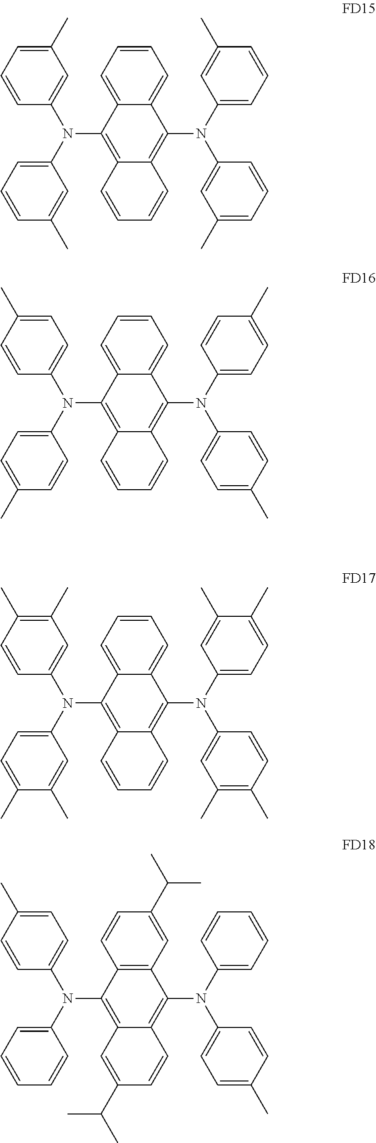 [SMILES: Cc1cccc(N(c2cccc(C)c2)c2c3ccccc3c(N(c3cccc(C)c3)c3cccc(C)c3)c3ccccc23)c1 ; Cc1ccc(N(c2ccc(C)cc2)c2c3ccccc3c(N(c3ccc(C)cc3)c3ccc(C)cc3)c3ccccc23)cc1 ; Cc1ccc(N(c2ccc(C)c(C)c2)c2c3ccccc3c(N(c3ccc(C)c(C)c3)c3ccc(C)c(C)c3)c3ccccc23)cc1C ; Cc1ccc(N(c2ccccc2)c2c3ccc(C(C)C)cc3c(N(c3ccccc3)c3ccc(C)cc3)c3ccc(C(C)C)cc23)cc1]

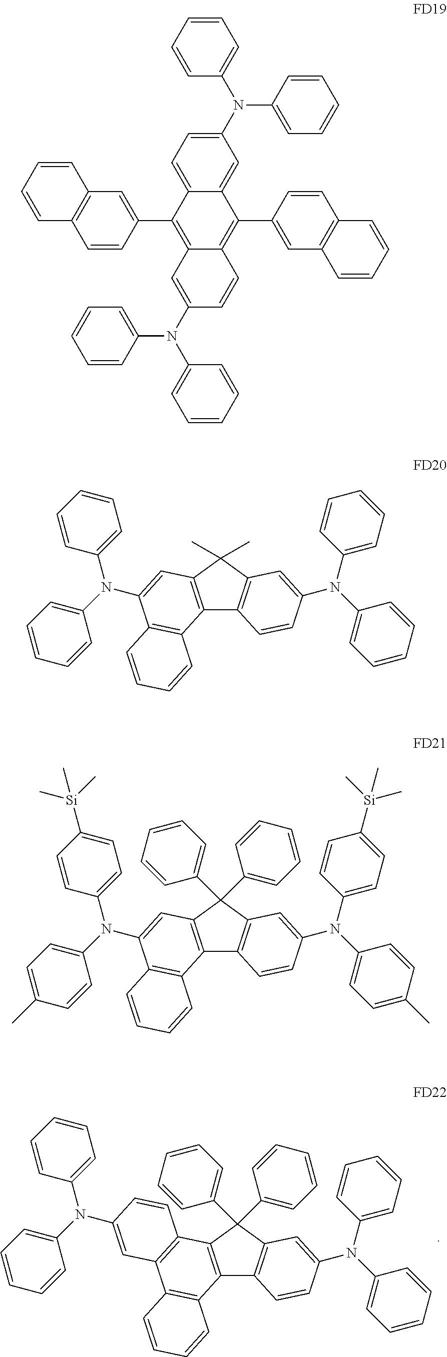

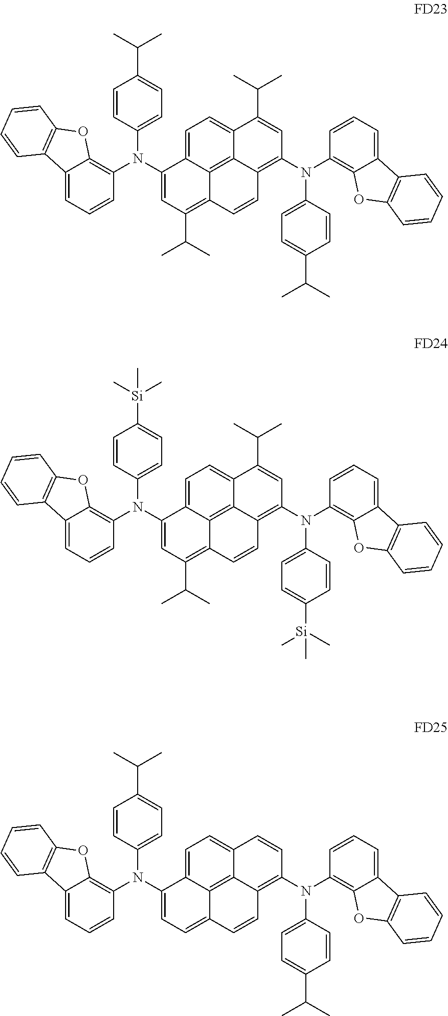 [SMILES: CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1 ; CC(C)c1cc(N(c2ccc([Si](C)(C)C)cc2)c2cccc3c2oc2ccccc23)c2ccc3c(C(C)C)cc(N(c4ccc([Si](C)(C)C)cc4)c4cccc5c4oc4ccccc45)c4ccc1c2c34 ; CC(C)c1ccc(N(c2ccc3ccc4c(N(c5ccc(C(C)C)cc5)c5cccc6c5oc5ccccc56)ccc5ccc2c3c54)c2cccc3c2oc2ccccc23)cc1]

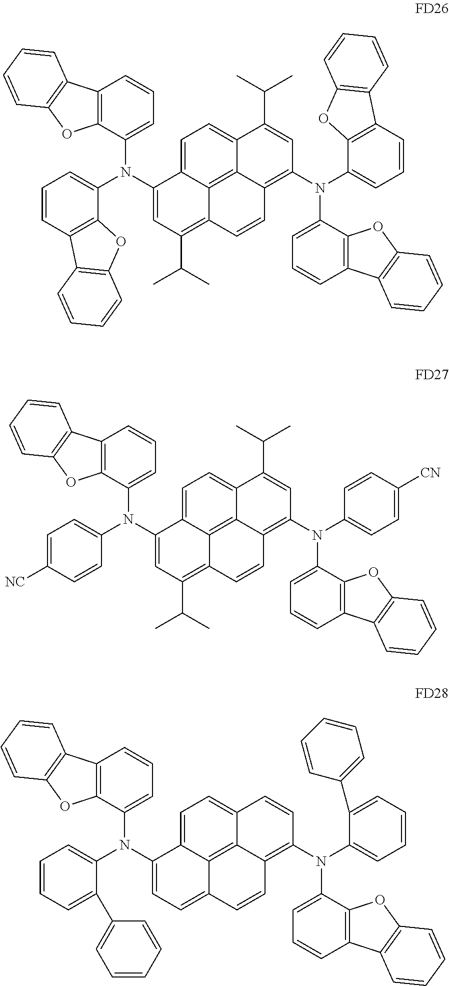 [SMILES: CC(C)c1cc(N(c2cccc3c2oc2ccccc23)c2cccc3c2oc2ccccc23)c2ccc3c(C(C)C)cc(N(c4cccc5c4oc4ccccc45)c4cccc5c4oc4ccccc45)c4ccc1c2c34 ; CC(C)c1cc(N(c2ccc(C#N)cc2)c2cccc3c2oc2ccccc23)c2ccc3c(C(C)C)cc(N(c4ccc(C#N)cc4)c4cccc5c4oc4ccccc45)c4ccc1c2c34 ; c1ccc(-c2ccccc2N(c2ccc3ccc4c(N(c5ccccc5-c5ccccc5)c5cccc6c5oc5ccccc56)ccc5ccc2c3c54)c2cccc3c2oc2ccccc23)cc1]

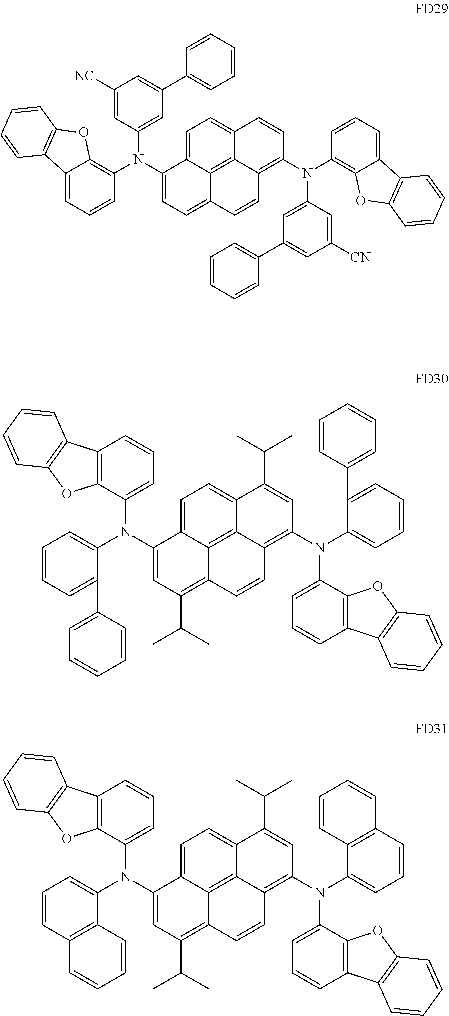 [SMILES: N#Cc1cc(-c2ccccc2)cc(N(c2ccc3ccc4c(N(c5cc(C#N)cc(-c6ccccc6)c5)c5cccc6c5oc5ccccc56)ccc5ccc2c3c54)c2cccc3c2oc2ccccc23)c1 ; CC(C)c1cc(N(c2ccccc2-c2ccccc2)c2cccc3c2oc2ccccc23)c2ccc3c(C(C)C)cc(N(c4ccccc4-c4ccccc4)c4cccc5c4oc4ccccc45)c4ccc1c2c34 ; CC(C)c1cc(N(c2cccc3ccccc23)c2cccc3c2oc2ccccc23)c2ccc3c(C(C)C)cc(N(c4cccc5ccccc45)c4cccc5c4oc4ccccc45)c4ccc1c2c34]

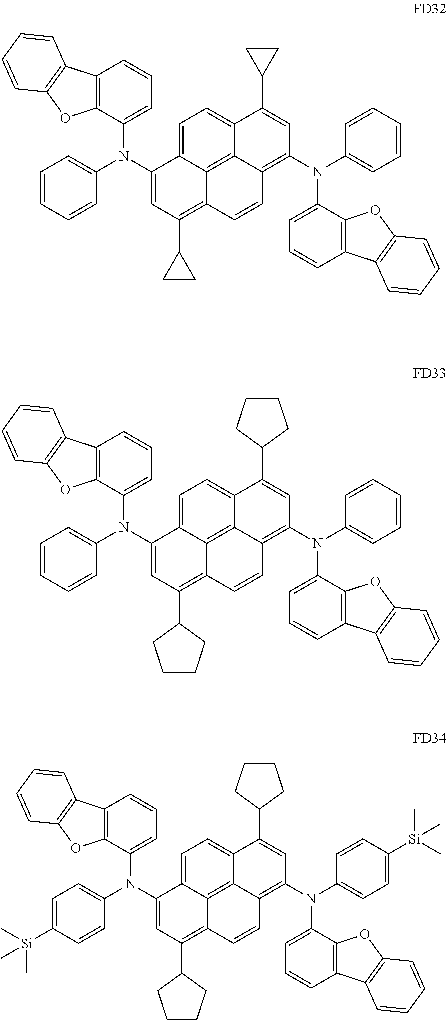

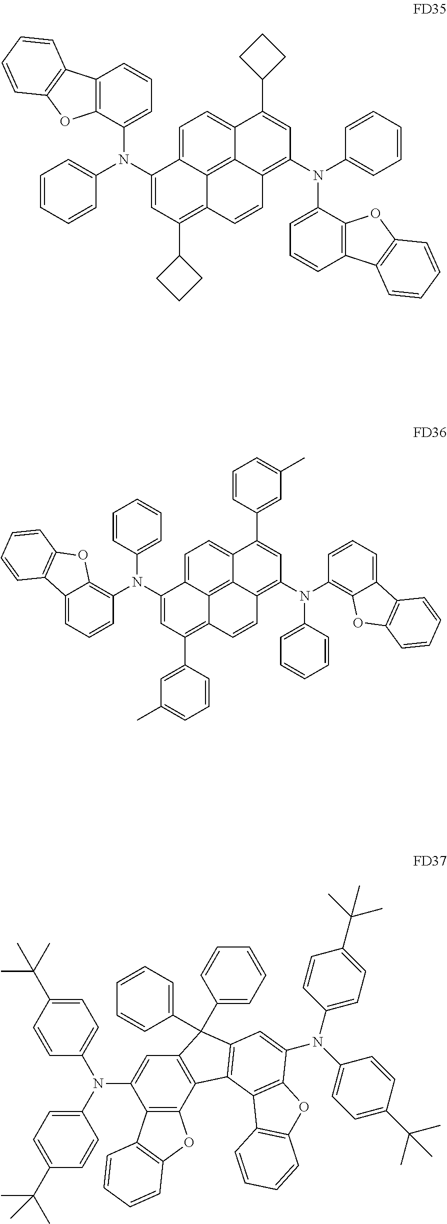 [SMILES: c1ccc(N(c2cc(C3CCC3)c3ccc4c(N(c5ccccc5)c5cccc6c5oc5ccccc56)cc(C5CCC5)c5ccc2c3c54)c2cccc3c2oc2ccccc23)cc1 ; Cc1cccc(-c2cc(N(c3ccccc3)c3cccc4c3oc3ccccc34)c3ccc4c(-c5cccc(C)c5)cc(N(c5ccccc5)c5cccc6c5oc5ccccc56)c5ccc2c3c45)c1 ; CC(C)(C)c1ccc(N(c2ccc(C(C)(C)C)cc2)c2cc3c(c4c2oc2ccccc24)-c2c(cc(N(c4ccc(C(C)(C)C)cc4)c4ccc(C(C)(C)C)cc4)c4c2oc2ccccc24)C3(c2ccccc2)c2ccccc2)cc1]

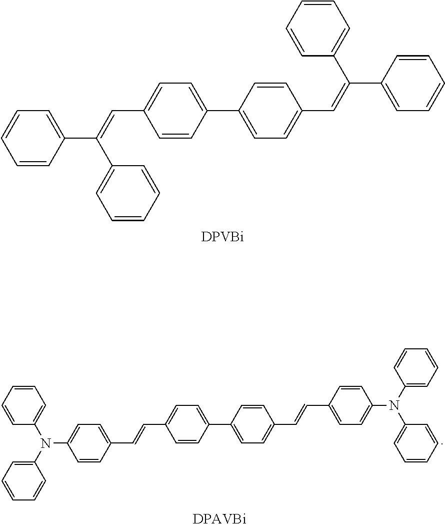

### Delayed Fluorescence Material

In one or more embodiments, the emission layer 130 may include a delayed fluorescence material.

In the present disclosure, the delayed fluorescence material may be selected from among compounds capable of emitting delayed fluorescence based on a delayed fluorescence emission mechanism.

The delayed fluorescence material included in the emission layer 130 may act as a host or a dopant, depending on the type or kind of other materials included in the emission layer 130.

In one or more embodiments, a difference between a triplet energy level (eV) of the delayed fluorescence material and a singlet energy level (eV) of the delayed fluorescence material may be in a range of about 0 eV and to about 0.5 eV. When the difference between the triplet energy level (eV) of the delayed fluorescence material and the singlet energy level (eV) of the delayed fluorescence material is satisfied within the range above, up-conversion from the triplet state to the singlet state of the delayed fluorescence materials may effectively occur, and thus, the light-emitting device 10 may have improved luminescence efficiency.

For example, in one or more embodiments, the delayed fluorescence material may include i) a material including at least one electron donor (e.g., a T electron-rich C₃-C₆₀ cyclic group, such as a carbazole group, and/or the like) and at least one electron acceptor (e.g., a sulfoxide group, a cyano group, a T electron-deficient nitrogen-containing C₁-C₆₀ cyclic group, and/or the like), and/or ii) a material including a C₈-C₆₀ polycyclic group in which two or more cyclic groups are condensed with each other while sharing boron (B).

Non-limiting examples of the delayed fluorescence material may include at least one of (e.g., selected from among) Compounds DF1 to DF14:

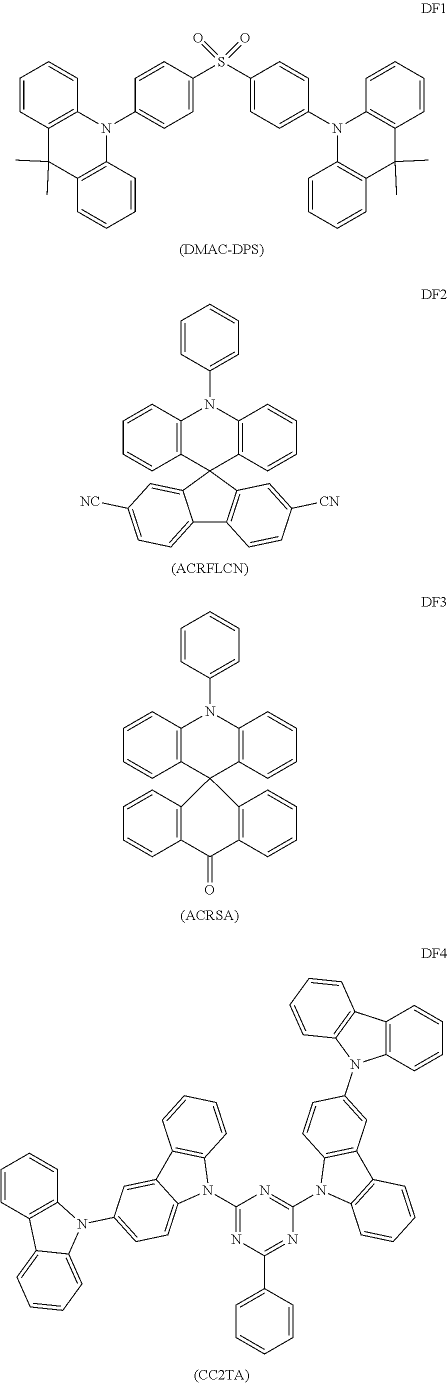

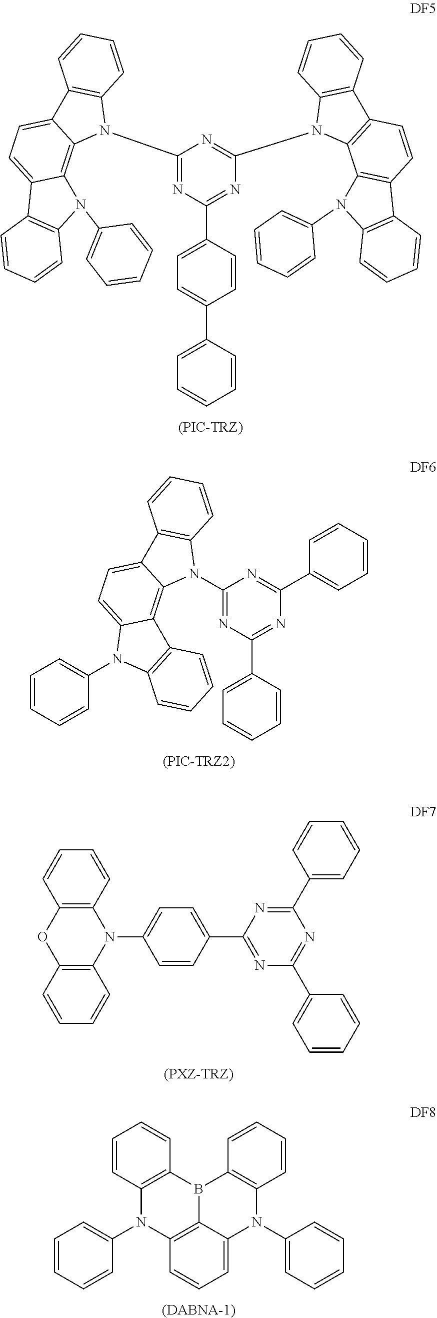

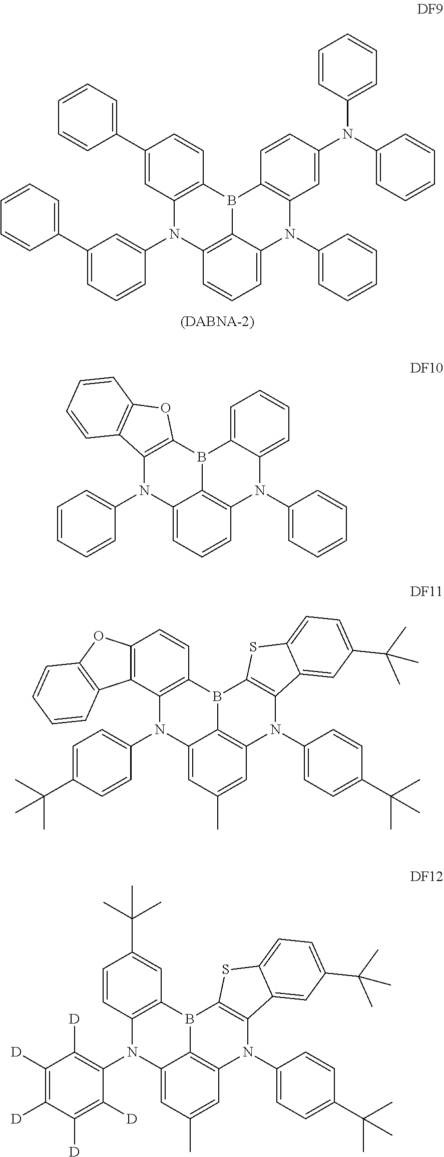

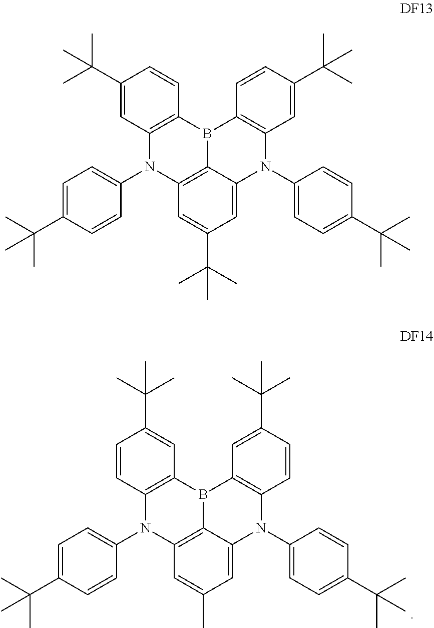

### Quantum Dots

In one or more embodiments, the emission layer 130 may include quantum dots.

In the disclosure, quantum dots refer to crystals of a semiconductor compound. Quantum dots may be to emit light of one or more suitable emission wavelengths depending on the size of crystals. Quantum dots may be to emit light of one or more suitable emission wavelengths by further adjusting a ratio of elements constituting the quantum dots.

A diameter of the quantum dots may be, for example, in a range of about 1 nm to about 10 nm. In the present disclosure, when dot, dots, or dot particles are spherical, “diameter” indicates a particle diameter or an average particle diameter, and when the particles are non-spherical, the “diameter” indicates a major axis length or an average major axis length. The diameter of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter is referred to as D50. D50 refers to the average diameter of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

The quantum dots may be synthesized by a wet chemical process, a metal organic chemical vapor deposition process, a molecular beam epitaxy process, or any process similar thereto.

The wet chemical process is a method including mixing a precursor material of a quantum dot with an organic solvent and then growing quantum dot particle crystals. When the crystals grow, the organic solvent naturally acts as a dispersant coordinated on the surface of the quantum dot crystals and controls the growth of the crystals so that the growth of quantum dot particles may be controlled or selected through a process which costs lower and is easier than vapor deposition methods, such as metal organic chemical vapor deposition (MOCVD) or molecular beam epitaxy (MBE).

The quantum dots may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Non-limiting examples of the Group II-VI semiconductor compound may include: a binary compound, such as CdS, CdSe, CdTe, ZnS, ZnSe, ZnTe, ZnO, HgS, HgSe, HgTe, MgSe, MgS, and/or the like; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and/or the like; a quaternary compound, such as CdZnSeS, CdZnSeTe, CdZnSTe, CdHgSeS, CdHgSeTe, CdHgSTe, HgZnSeS, HgZnSeTe, HgZnSTe, and/or the like; or any combination thereof.

Non-limiting examples of the Group III-V semiconductor compound may include: a binary compound, such as GaN, GaP, GaAs, GaSb, AlN, AIP, AIAs, AISb, InN, InP, InAs, InSb, and/or the like; a ternary compound, such as GaNP, GaNAs, GaNSb, GaPAs, GaPSb, AlNP, AlNAs, AlNSb, AIPAs, AIPSb, InGaP, InNP, InAlP, InNAs, InNSb, InPAs, InPSb, and/or the like; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and/or the like; or any combination thereof. In one or more embodiments, the Group III-V semiconductor compound may further include a Group II element. Non-limiting examples of the Group III-V semiconductor compound further including a Group II element may include InZnP, InGaZnP, InAlZnP, and/or the like.

Non-limiting examples of the Group III-VI semiconductor compound are: a binary compound, such as GaS, Ga₂Se₃, GaTe, InS, InSe, In₂S₃, In₂Se₃, InTe, and/or the like; a ternary compound, such as InGaS₃, InGaSe₃, and/or the like; or any combination thereof.

Non-limiting examples of the Group 1-III-VI semiconductor compound may include: a ternary compound, such as AgInS, AgInS₂, AgInSe₂, AgGaS, AgGaS₂, AgGaSe₂, CuInS, CuInS₂, CuInSe₂, CuGaS₂, CuGaSe₂, CuGaO₂, AgGaO₂, AgAlO₂, and/or the like; a quaternary compound, such as AgInGaS₂, AgInGaSe₂, and/or the like; or any combination thereof.

Non-limiting examples of the Group IV-VI semiconductor compound may include: a binary compound, such as SnS, SnSe, SnTe, PbS, PbSe, and/or PbTe; a ternary compound, such as SnSeS, SnSeTe, SnSTe, PbSeS, PbSeTe, PbSTe, SnPbS, SnPbSe, SnPbTe, and/or the like; a quaternary compound, such as SnPbSSe, SnPbSeTe, SnPbSTe, and/or the like; or any combination thereof.

Non-limiting examples of the Group IV element or compound may include: a single element, such as Si, Ge, and/or the like; a binary compound, such as SiC, SiGe, and/or the like; or any combination thereof.

Each element included in a multi-element compound, such as the binary compound, the ternary compound, and the quaternary compound, may be present at a substantially uniform concentration or non-uniform concentration in a particle. For example, the formulae above refer to types (kinds) of elements included in the compound, and the element ratios within the compound may vary. For example, AgInGaS₂ refers to AgInₓGa₁₋ₓS₂ (where x is a real number between 0 and 1).

In one or more embodiments, the quantum dots may have a single structure in which the concentration of each element in the quantum dots is substantially uniform, or a core-shell dual structure. For example, materials included in the core and materials included in the shell may be different from each other.

The shell of the quantum dots may act as a protective layer that prevents chemical degeneration of the core to maintain semiconductor characteristics, and/or as a charging layer that imparts electrophoretic characteristics to the quantum dots. The shell may be single-layered or multi-layered. The interface between the core and a first shell may have a concentration gradient in which the concentration of an element existing in the first shell decreases toward the center of the core.

Examples of the shell of the quantum dot may be an oxide of a metal, metalloid, or non-metal, a semiconductor compound, and/or a (e.g., any suitable) combination thereof. Non-limiting examples of the oxide of metal or non-metal are: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, and/or the like; a ternary compound, such as MgAl₂O₄, CoFe₂O₄, NiFe₂O₄, CoMn₂O₄, and/or the like; or any combination thereof. Examples of the semiconductor compound are: as described herein, Group III-VI semiconductor compounds; Group II-VI semiconductor compounds; Group III-V semiconductor compounds; Group III-VI semiconductor compounds; Group 1-III-VI semiconductor compounds; Group IV-VI semiconductor compounds; or any combination thereof. For example, the semiconductor compound suitable as a shell may include CdS, CdSe, CdTe, ZnS, ZnSe, ZnTe, ZnSeS, ZnTeS, GaAs, GaP, GaS, GaSe, AgGaS, AgGaS₂, GaSb, HgS, HgSe, HgTe, InAs, InP, InGaP, InSb, AIAs, AIP, AIS b, or any combination thereof.

Each element included in the multi-element compound, such as the binary compound and the ternary compound, may be present in the particle at a substantially uniform or non-uniform concentration. For example, the formulae above refer to types (kinds) of elements included in the compound, and the element ratios within the compound may vary.

A full width of half maximum (FWHM) of an emission spectrum of the quantum dots may be about 45 nm or less, for example, about 40 nm or less, and for example, about 30 nm or less, and within these ranges, the color purity or color reproducibility of the quantum dots may be improved. In addition, because light emitted through the quantum dots is emitted in all directions, the wide viewing angle may be improved.

In addition, the quantum dots may be nanoparticles, nanotubes, nanowires, nanofibers, nanoplates, and/or the like, for example, in the form of spherical particles, pyramidal particles, multi-arm particles, or cubic particles.

Because an energy band gap of the quantum dot may be controlled or selected by adjusting the size of the quantum dots or the ratio of elements in the quantum dot compound, light of one or more suitable wavelengths may be obtained from a quantum dot-containing emission layer. Therefore, by using the aforementioned quantum dots (using quantum dots of different sizes or having different element ratios in the quantum dot compound), a light-emitting device emitting light of one or more suitable wavelengths may be implemented. For example, the size of the quantum dots and/or the ratio of elements in the quantum dot compound may be controlled and selected enable the quantum dots to emit red light, green light, and/or blue light. In addition, the quantum dots with suitable sizes/diameters may be configured to emit white light by combination of light of one or more suitable colors.

### Electron Transport Region 140

The electron transport region 140 may have: i) a single-layer structure including (e.g., consisting of) a single layer including (e.g., consisting of) a single material, ii) a single-layer structure including (e.g., consisting of) a single layer including (e.g., consisting of) multiple materials that are different from each other, or iii) a multi-layer structure including multiple layers including multiple materials that are different from each other.

The electron transport region 140 may include a buffer layer, a hole blocking layer, an electron control layer, an electron transport layer, an electron injection layer, or any combination thereof.

For example, the electron transport region 140 may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein constituent layers of each structure may be sequentially stacked from the emission layer 130 in the stated order.

In one or more embodiments, the electron transport region 140 (e.g., the buffer layer, the hole blocking layer, the electron control layer, or the electron transport layer in the electron transport region 140) may include a metal-free compound including at least one T electron-deficient nitrogen-containing C₁-C₆₀ cyclic group.

For example, in one or more embodiments, the electron transport region 140 may include a compound represented by Formula 601:

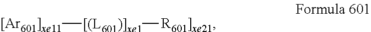

wherein, in Formula 601,
Ar₆₀₁ and L₆₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each independently be the same as described with respect to Q₁,
xe21 may be 1, 2, 3, 4, or 5, and
at least one selected from among Ar₆₀₁, L₆₀₁, and R₆₀₁ may each independently be a T electron-deficient nitrogen-containing C₁-C₆₀ cyclic group unsubstituted or substituted with at least one R₁₀ₐ.

For example, in one or more embodiments, if (e.g., when) xe11 in Formula 601 is 2 or more, two or more of Ar₆₀₁(s) may be linked to each other via a single bond.

In one or more embodiments, Ar₆₀₁ in Formula 601 may be an anthracene group that is unsubstituted or substituted with at least one R₁₀ₐ.

In one or more embodiments, the electron transport region 140 may include a compound represented by Formula 601-1:

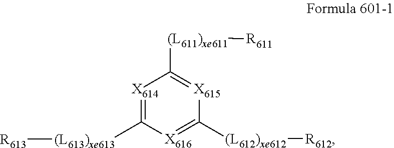

wherein, in Formula 601-1,
X₆₁₄ may be N or C(R₆₁₄), X₆₁₅ may be N or C(R₆₁₅), and X₆₁₆ may be N or C(R₆₁₆), wherein at least one selected from among X₆₁₄ to X₆₁₆ may be N,
L₆₁₁ to L₆₁₃ may each independently be the same as described with respect to L₆₀₁,
xe611 to xe613 may each independently be the same as described with respect to xe1,
R₆₁₁ to R₆₁₃ may each independently be the same as described with respect to R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

In one or more embodiments, xe1 and xe611 to xe613 in Formulae 601 and 601-1 may each independently be 0, 1, or 2.

In one or more embodiments, the electron transport region may include: at least one of (e.g., selected from among) Compounds ET1 to ET45; 2,9-dimethyl-4,7-diphenyl-1,10-phenanthroline (BCP); 4,7-diphenyl-1,10-phenanthroline (Bphen); tris(8-hydroxyquinolinato)aluminum (Alq₃); bis(2-methyl-8-quinolinolato-N1,O8)-(1,1′-biphenyl-4-olato)aluminum (BAlq); 3-(4-biphenyl)-4-phenyl-5-tert-butylphenyl-1,2,4-triazole (TAZ); 4-(naphthalen-1-yl)-3,5-diphenyl-4H-1,2,4-triazole (NTAZ); or any combination thereof:

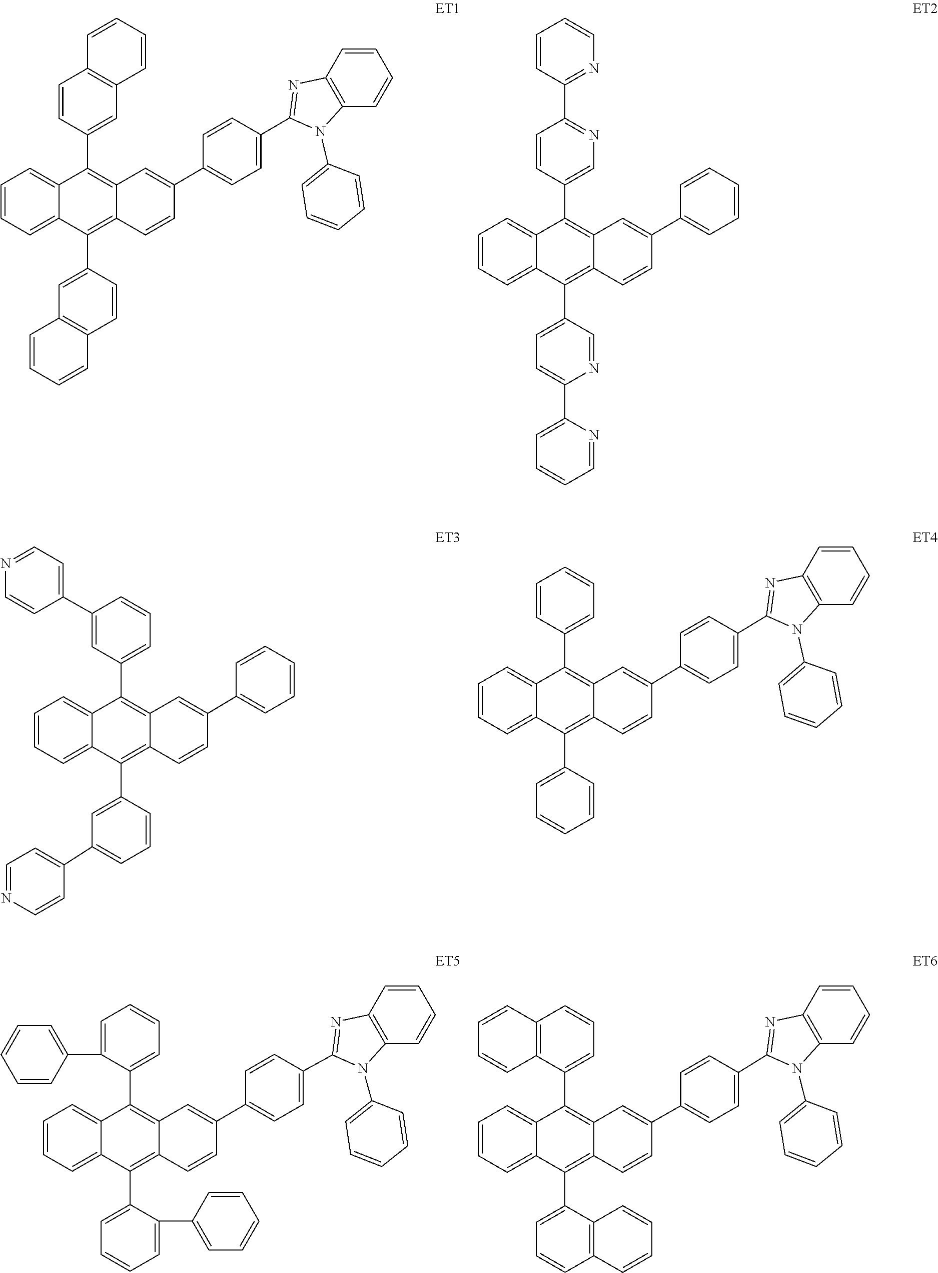

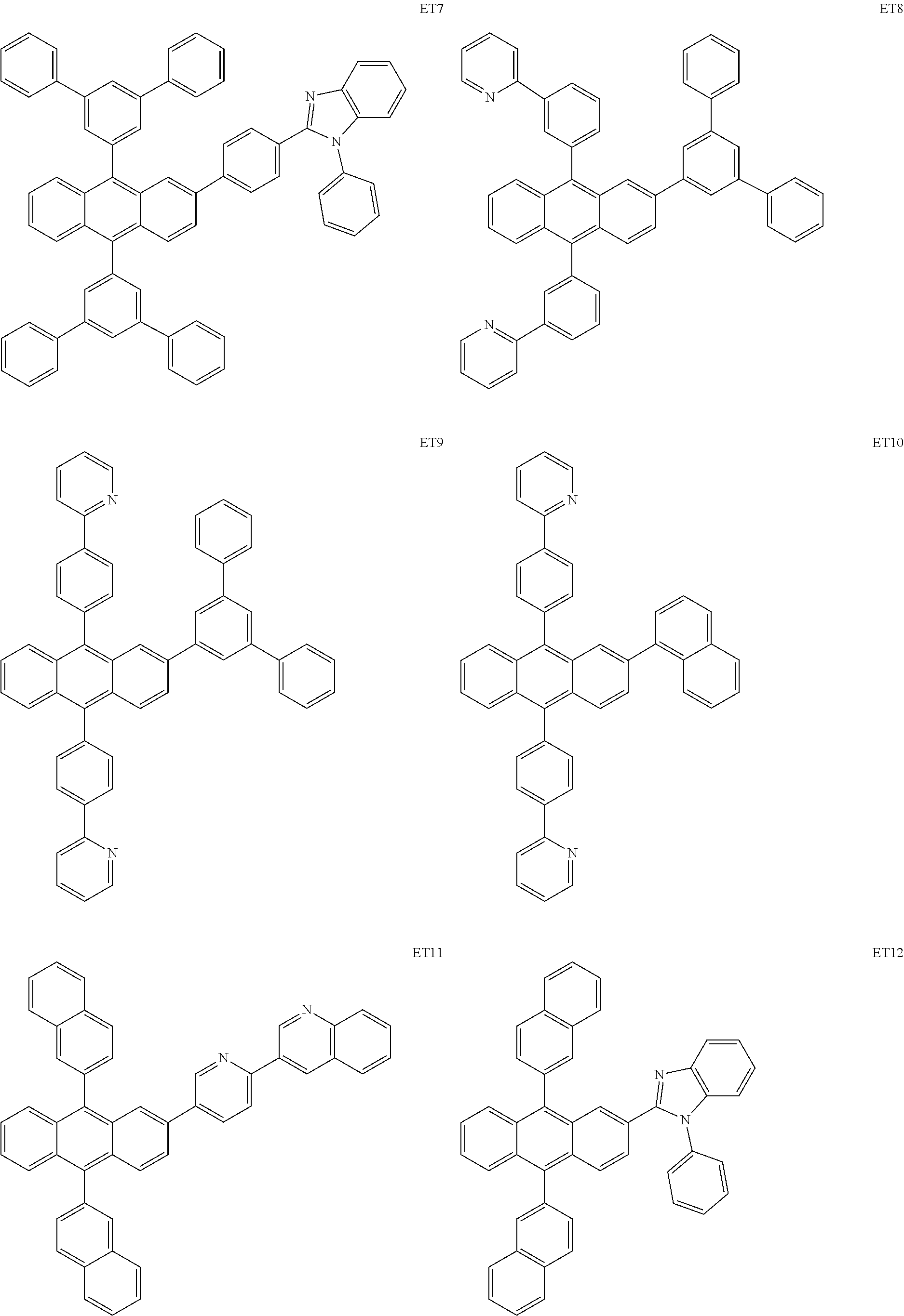

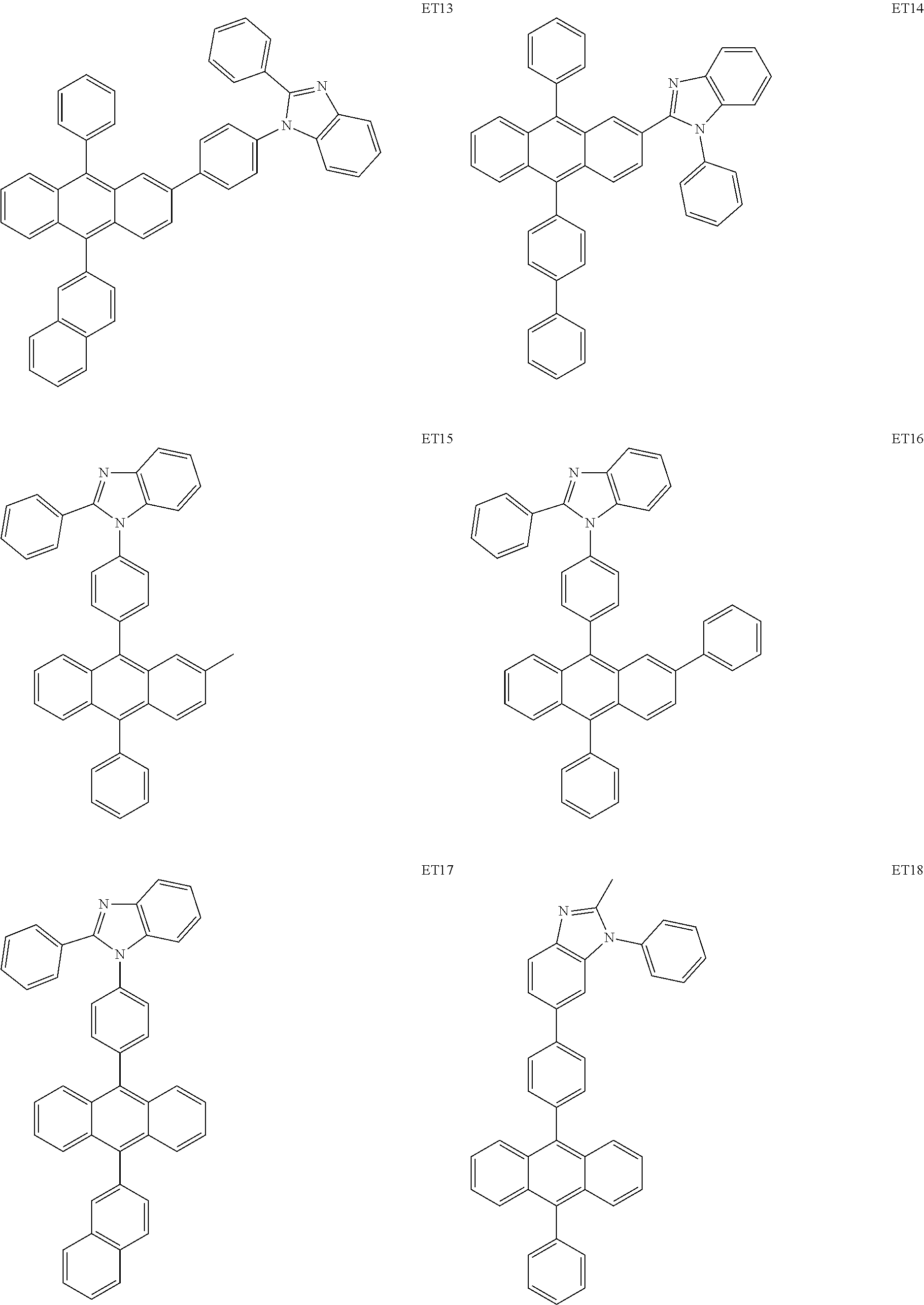 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

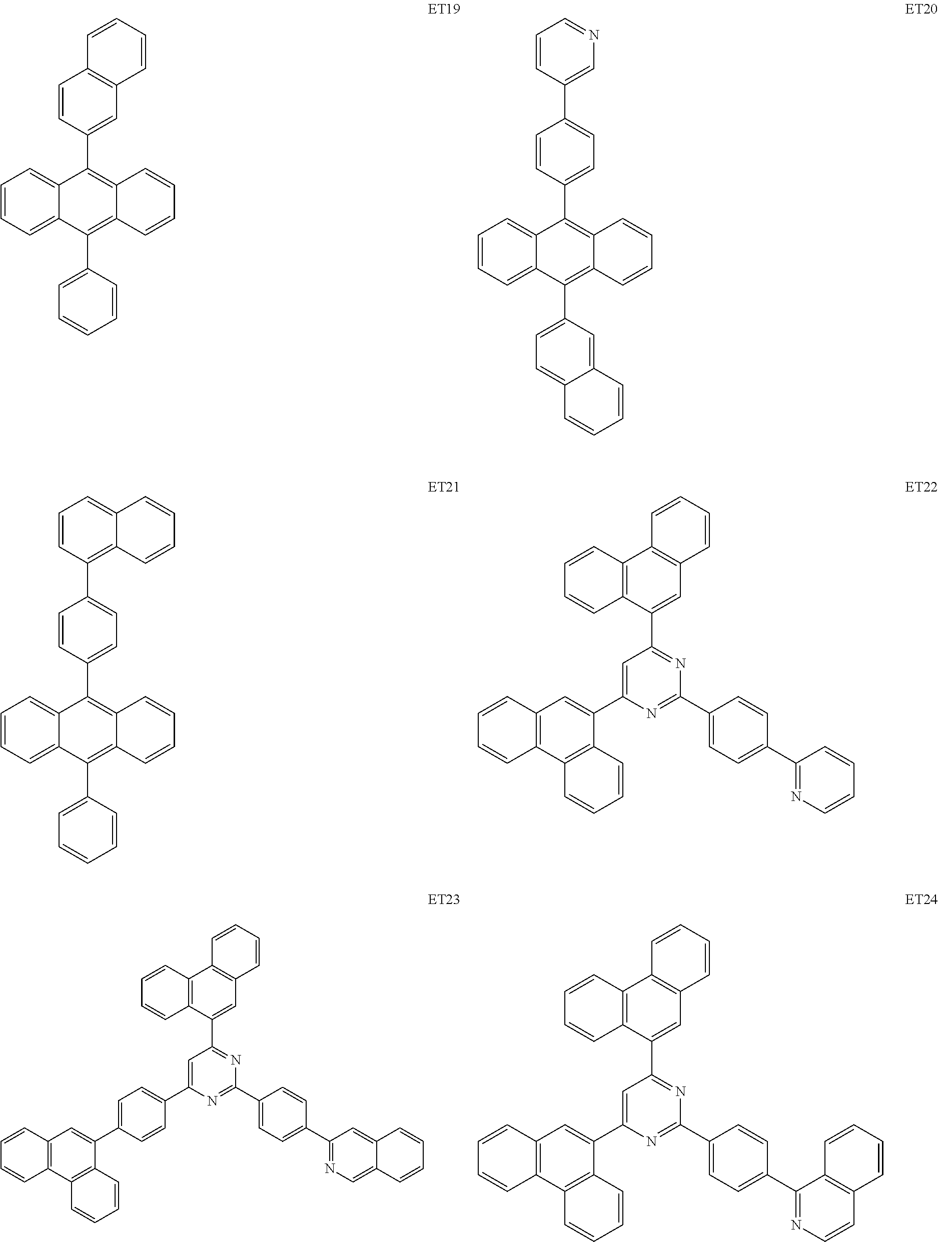

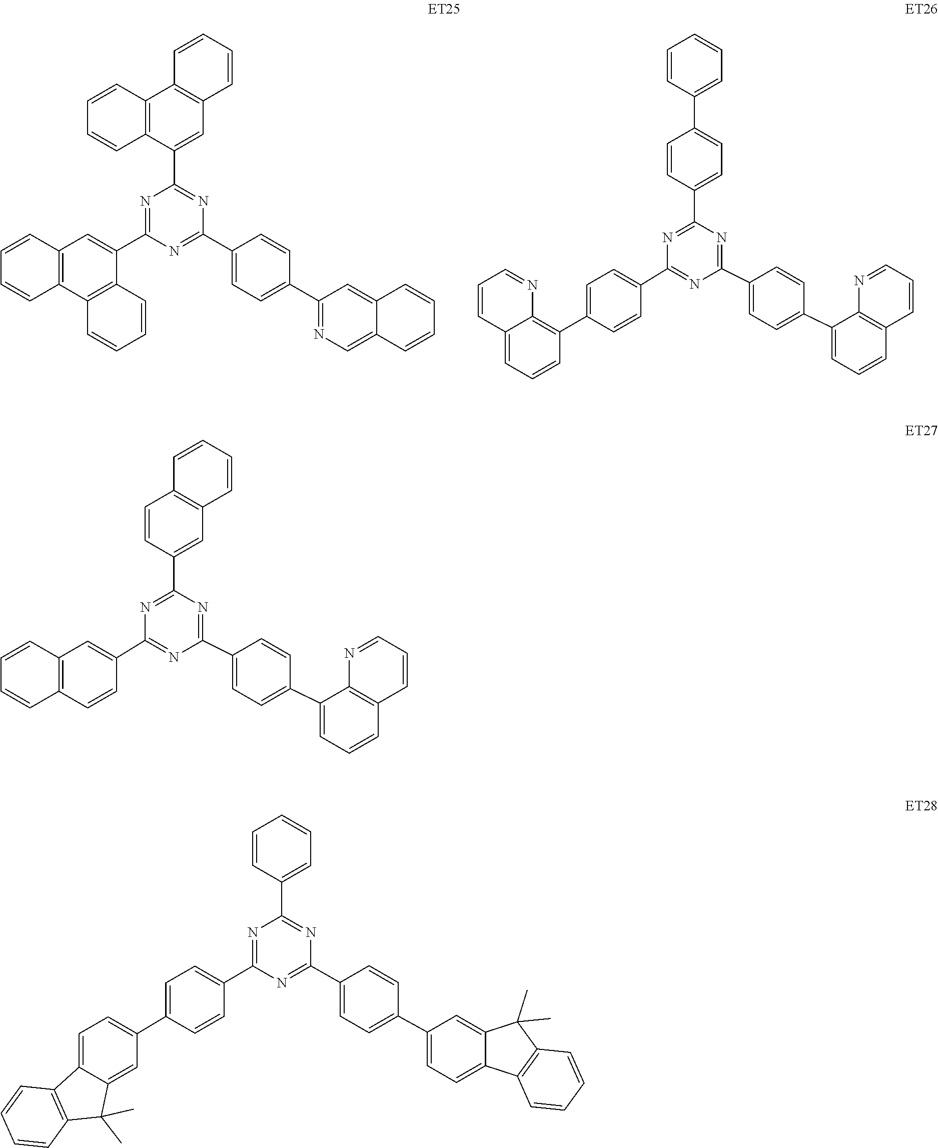

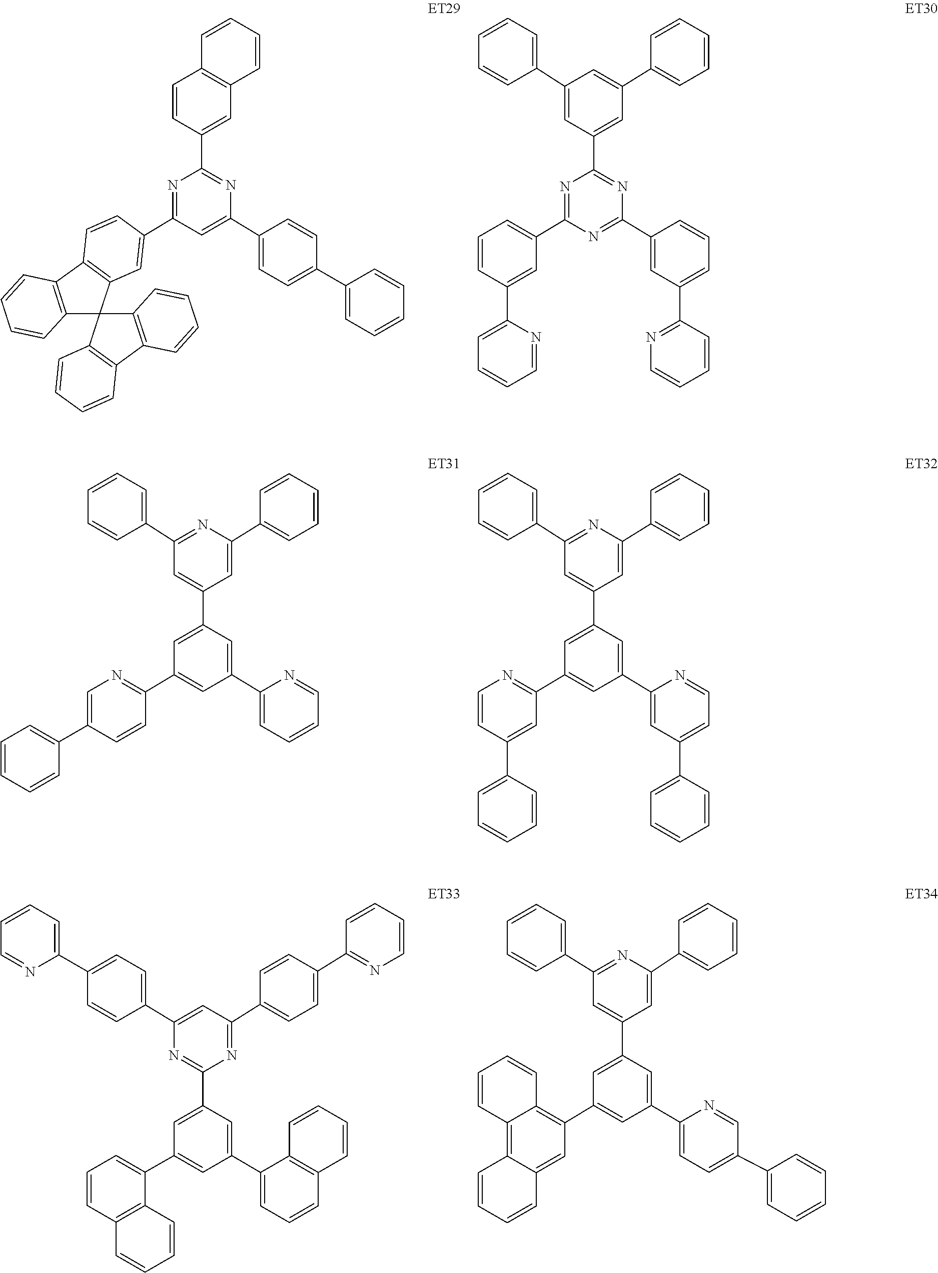

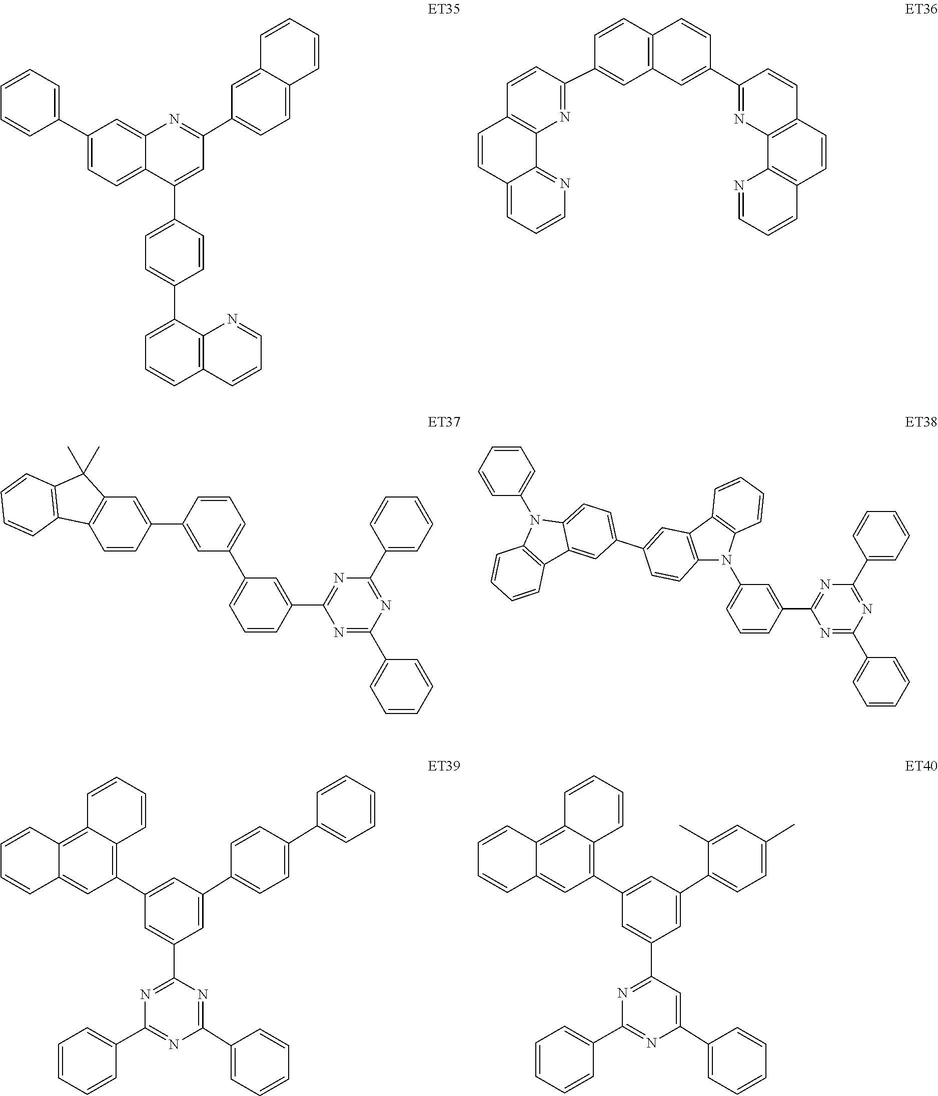 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

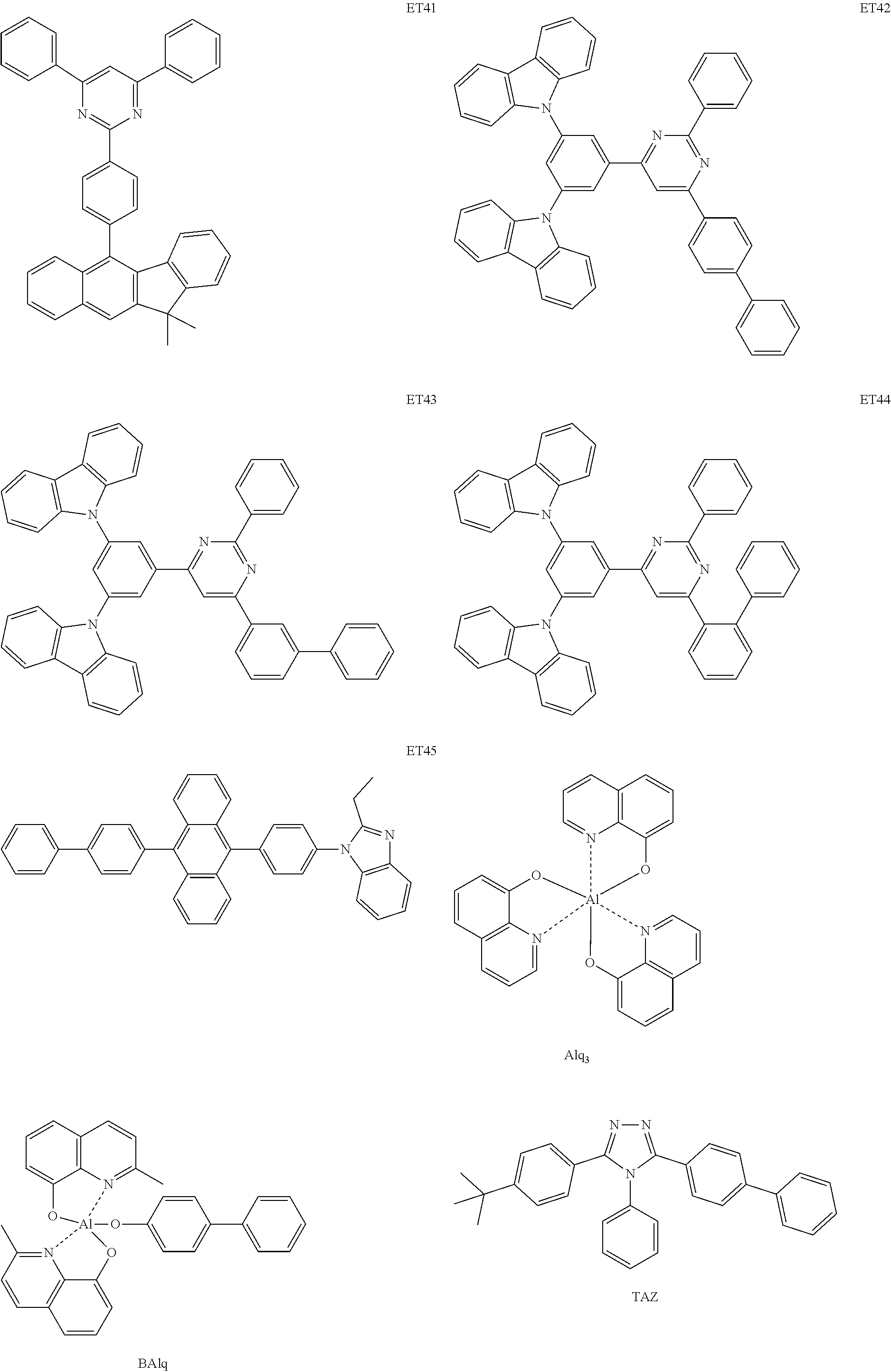

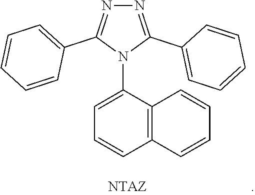 [SMILES: c1ccc(-c2nnc(-c3ccccc3)n2-c2cccc3ccccc23)cc1]

A thickness of the electron transport region 140 may be in a range of about 100 Å to about 5,000 Å, for example, about 160 Å to about 4,000 Å. When the electron transport region 140 includes a buffer layer, a hole blocking layer, an electron control layer, an electron transport layer, or any combination thereof, a thickness of the buffer layer, the hole blocking layer, or the electron control layer may each independently be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thicknesses of the buffer layer, the hole blocking layer, the electron control layer, the electron transport layer, and/or the electron transport region 140 are within these ranges, satisfactory electron transporting characteristics may be obtained without a substantial increase in driving voltage.

In one or more embodiments, the electron transport region 140 (e.g., an electron transport layer in the electron transport region) may further include, in addition to one or more of the aforementioned materials, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the metal ion of the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

In one or more embodiments, the metal-containing material may include a Li complex. The Li complex may include, for example, Compound ET-D1 (LiQ) or ET-D2:

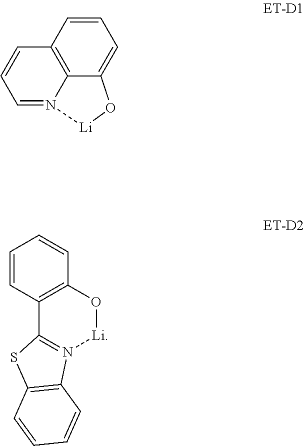

In one or more embodiments, the electron transport region 140 may include an electron injection layer that facilitates the injection of electrons from the second electrode 150. The electron injection layer may directly contact the second electrode 150.

The electron injection layer may have: i) a single-layer structure including (e.g., consisting of) a single layer including (e.g., consisting of) a single material, ii) a single-layer structure including (e.g., consisting of) a single layer including (e.g., consisting of) multiple layers that are different from each other, or iii) a multi-layer structure including multiple layers including multiple materials that are different from each other.

In one or more embodiments, the electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (e.g., fluorides, chlorides, bromides, or iodides), or tellurides of the alkali metal, the alkaline earth metal, and the rare earth metal, respectively, or any combination thereof.

The alkali metal-containing compound may include: alkali metal oxides, such as Li₂O, Cs₂O, and/or K₂O; alkali metal halides, such as LiF, NaF, CsF, KF, LiI, NaI, CsI, and/or KI; or any combination thereof. The alkaline earth metal-containing compound may include an alkaline earth metal compound, such as BaO, SrO, CaO, BaₓSr₁₋ₓO (wherein x is a real number satisfying 0<x<1), BaₓCa₁₋ₓO (wherein x is a real number satisfying 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal tellurides. Non-limiting examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, E r₂Te₃, Tm₂Te₃, Y b₂Te₃, Lu₂Te₃, and/or the like.

The alkali metal complex, the alkaline earth-metal complex, and the rare earth metal complex may include i) one of metal ions of the alkali metal, one of metal ions of the alkaline earth metal, and one of metal ions of the rare earth metal, respectively, and ii) a ligand bonded to the metal ion (e.g., the respective metal ion), for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

In one or more embodiments, the electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (e.g., the compound represented by Formula 601).

In one or more embodiments, the electron injection layer may include (e.g., consist of) i) an alkali metal-containing compound (e.g., an alkali metal halide), or ii) a) an alkali metal-containing compound (e.g., an alkali metal halide), and b) an alkali metal, an alkaline earth metal, a rare earth metal, or any combination thereof. In one or more embodiments, the electron injection layer may be a KI:Yb co-deposited layer, an RbI:Yb co-deposited layer, a LiF:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be uniformly (e.g., substantially uniformly) or non-uniformly dispersed in a matrix including the organic material.

A thickness of the electron injection layer may be in a range of about 1 Å to about 100 Å, and, for example, about 3 Å to about 90 Å. When the thickness of the electron injection layer is within these ranges, satisfactory electron injection characteristics may be obtained without a substantial increase in driving voltage.

### Second Electrode 150

The second electrode 150 may be arranged on the electron transport region 140. The second electrode 150 may be a cathode, which is an electron injection electrode, and as a material for forming the second electrode 150, a metal, an alloy, an electrically conductive compound, or any combination thereof, each having a low-work function, may be used.

The second electrode 150 may include Li, Ag, Mg, Al, Al—Li, Ca, Mg—In, Mg—Ag, Yb, Ag—Yb, ITO, IZO, or any combination thereof. The second electrode 150 may be a transmissive electrode, a semi-transmissive electrode, or a reflective electrode.

The second electrode 150 may have a single-layer structure or a multi-layer structure including multiple layers.

### Capping Layer

In one or more embodiments, a first capping layer may be arranged outside (e.g., on) the first electrode 110, and/or a second capping layer may be arranged outside (e.g., on) the second electrode 150. For example, in one or more embodiments, the light-emitting device 10 may have a structure in which the first capping layer, the first electrode 110, the interlayer, and the second electrode 150 are sequentially stacked in the stated order, a structure in which the first electrode 110, the interlayer, the second electrode 150, and the second capping layer are sequentially stacked in the stated order, or a structure in which the first capping layer, the first electrode 110, the interlayer, the second electrode 150, and the second capping layer are sequentially stacked in the stated order.

In one or more embodiments, light generated in the emission layer 130 of the light-emitting device 10 may pass through the first electrode 110, which is a semi-transmissive electrode or a transmissive electrode, and through the first capping layer to the outside. In one or more embodiments, light generated in the emission layer 130 of the light-emitting device 10 may pass through the second electrode 150, which is a semi-transmissive electrode or a transmissive electrode, and through the second capping layer to the outside.

The first capping layer and the second capping layer may increase external emission efficiency according to the principle of constructive interference. Accordingly, the light extraction efficiency of the light-emitting device 10 may be increased, and accordingly, the luminescence efficiency of the light-emitting device 10 may be improved.

Each of the first capping layer and the second capping layer may include a material having a refractive index of about 1.2 or more (e.g., at 460 nm).

The first capping layer and the second capping layer may each independently be an organic capping layer including organic materials, an inorganic capping layer including inorganic materials, or an organic-inorganic composite capping layer including organic materials and inorganic materials.

At least one of the first capping layer and/or the second capping layer may (e.g., the first capping layer and the second capping layer may each independently) include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may each optionally be substituted with a substituent including O, N, S, Se, Si, F, Cl, Br, I, or any combination thereof. In one or more embodiments, at least one of the first capping layer and/or the second capping layer may (e.g., the first capping layer and the second capping layer may each independently) include an amine group-containing compound.

In one or more embodiments, at least one of the first capping layer and/or the second capping layer may (e.g., the first capping layer and the second capping layer may each independently) include a compound represented by Formula 201, a compound represented by Formula 202, or any combination thereof.

In one or more embodiments, at least one of the first capping layer and/or the second capping layer may (e.g., the first capping layer and the second capping layer may each independently) include: one of (e.g., selected from among) Compounds HT28 to HT33; one of (e.g., selected from among) Compounds CP1 to CP6; β-NPB; or any combination thereof:

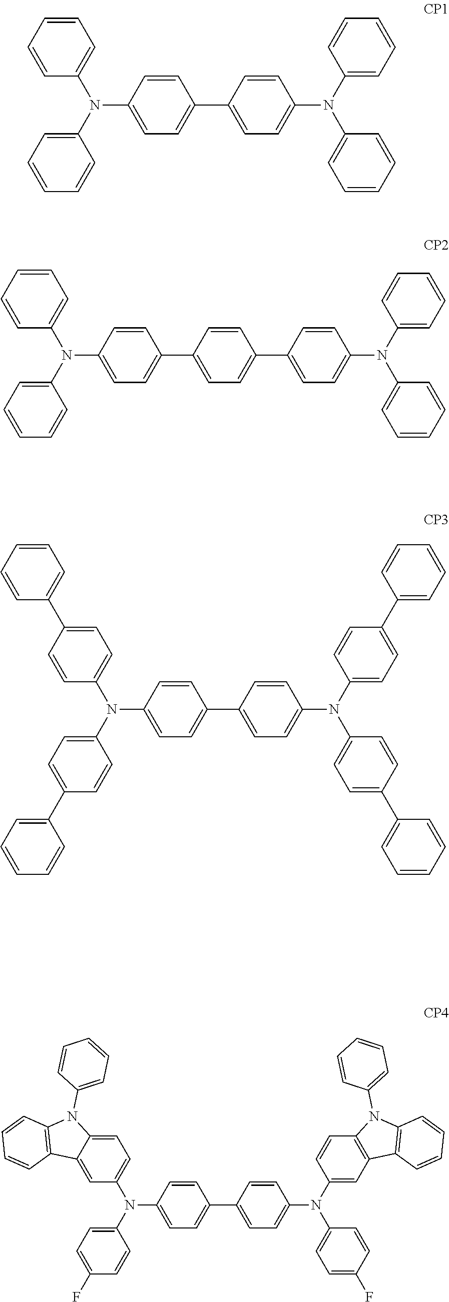 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

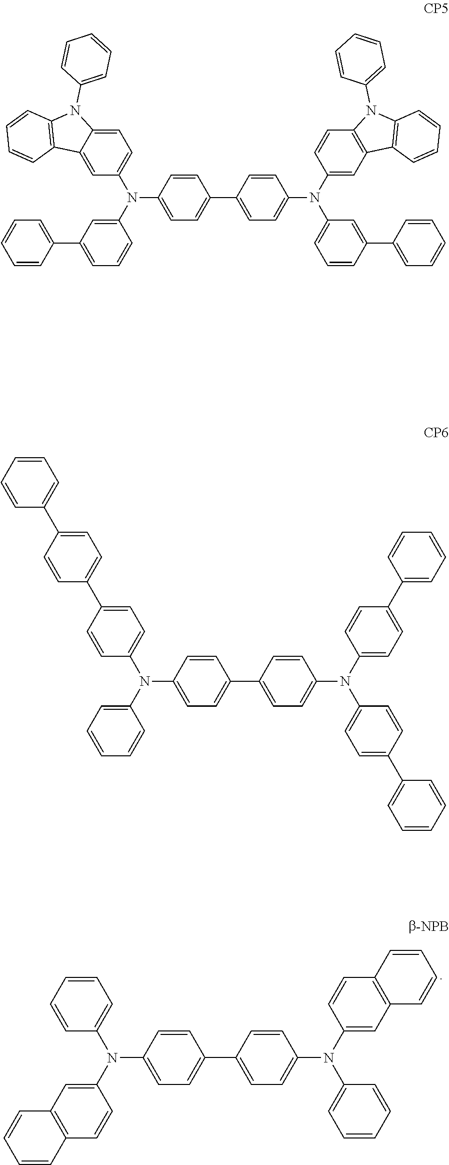 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

In one or more embodiments, the electronic apparatus may further include a film. The film may be, for example, an optical member (or a light control element) (e.g., a color filter, a color conversion member, a capping layer, a light extraction efficiency enhancement layer, a selective light absorbing layer, a polarizing layer, a quantum dot-containing layer, or like), a light blocking member (e.g., a light reflective layer, a light absorbing layer, and/or the like), a protective member (e.g., an insulating layer, a dielectric layer, and/or the like).

### Electronic Apparatus

The light-emitting device may be included in one or more suitable electronic apparatuses. For example, the electronic apparatus including the light-emitting device may be a display apparatus, an authentication apparatus, and/or the like.

In one or more embodiments, the electronic apparatus (e.g., a display apparatus) may further include, in addition to the light-emitting device, i) a color filter, ii) a color conversion layer, or iii) a color filter and a color conversion layer. The color filter and/or the color conversion layer may be arranged in at least one direction in which light emitted from the light-emitting device travels. For example, in one or more embodiments, light emitted from the light-emitting device may be blue light or white light (e.g., combined white light). For a description of the light-emitting device, reference may be made to the present disclosure. In one or more embodiments, the color conversion layer may include quantum dots. The quantum dots may be, for example, the aforementioned quantum dots.

The electronic apparatus may include a first substrate. The first substrate may include a plurality of subpixel areas, the color filter may include a plurality of color filter areas respectively corresponding to the plurality of subpixel areas, and the color conversion layer may include a plurality of color conversion areas respectively corresponding to the plurality of subpixel areas.

A pixel-defining film (or referred as to “a pixel-defining layer”) may be arranged among the plurality of subpixel areas to define each of the subpixel areas.

In one or more embodiments, the color filter may further include a plurality of color filter areas and light-shielding patterns thereon, and the color conversion layer may further include a plurality of color conversion areas and light-shielding patterns thereon.

The plurality of color filter areas (or the plurality of color conversion areas) may include: a first area configured to emit first color light; a second area configured to emit second color light; and/or a third area configured to emit third color light, wherein the first color light, the second color light, and/or the third color light may have different maximum emission wavelengths from one another. In one or more embodiments, the first color light may be red light, the second color light may be green light, and the third color light may be blue light. In one or more embodiments, the plurality of color filter areas (or the plurality of color conversion areas) may include quantum dots. For example, the first area may include red quantum dots to emit red light, the second area may include green quantum dots to emit green light, and the third area may not include (e.g., may exclude) quantum dots. For a description of the quantum dots, reference may be made to the present disclosure. Each of the first area, the second area, and/or the third area may further include a scatter.

For example, in one or more embodiments, in the light-emitting device emitting first light, the first area may be to absorb the first light to emit first-first color light, the second area may be to absorb the first light to emit second-first color light, and the third area may be to absorb the first light to emit third-first color light. Here, the first-first color light, the second-first color light, and the third-first color light may have different maximum emission wavelengths from one another. For example, the first light may be blue light, the first-first color light may be red light, the second-first color light may be green light, and the third-first color light may be blue light.

In one or more embodiments, the electronic apparatus may further include a thin-film transistor, in addition to the aforementioned light-emitting device. The thin-film transistor may include a source electrode, a drain electrode, and an activation layer, wherein one selected from among the source electrode and the drain electrode may be electrically connected to the first electrode 110 or the second electrode 150 of the light-emitting device 10.

The thin-film transistor may further include a gate electrode, a gate insulating film, and/or the like.

The activation layer may include crystalline silicon, amorphous silicon, an organic semiconductor, an oxide semiconductor, and/or the like.

In one or more embodiments, the electronic apparatus may further include a sealing portion for sealing the light-emitting device. The sealing portion may be arranged between the color filter and/or the color conversion layer and the light-emitting device 10. The sealing portion allows light from the light-emitting device to be extracted to the outside, and concurrently (e.g., simultaneously) prevents ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin-film encapsulation layer, the electronic apparatus may be flexible.

In one or more embodiments, various functional layers may be additionally arranged on the sealing portion, in addition to the color filter and/or the color conversion layer, according to the use of the electronic apparatus. Non-limiting examples of the functional layers may include a touch screen layer and a polarizing layer. The touch screen layer may be a pressure-sensitive touch screen layer, a capacitive touch screen layer, or an infrared touch screen layer.

The authentication apparatus may be, for example, a biometric authentication apparatus that authenticates an individual by using biometric information of a living body (e.g., fingertips, pupils, and/or the like). The authentication apparatus may further include, in addition to the light-emitting device, a biometric information collector.

The electronic apparatus may be applied to one or more of displays, light sources, lighting, personal computers (e.g., mobile personal computers), mobile phones, digital cameras, electronic organizers, electronic dictionaries, electronic game machines, medical instruments (e.g., electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (e.g., meters for a vehicle, an aircraft, and a vessel), projectors, and/or the like.

### Electronic Equipment

The light-emitting device may be included in one or more suitable types (kinds) of electronic equipment.

For example, the electronic equipment including the light-emitting device may be at least one of a flat panel display, a curved display, a computer monitor, a medical monitor, a television, a billboard, a light for indoor lighting, a light for outdoor lighting, a light for signaling, a head-up display, a fully transparent display, a partially transparent display, a flexible display, a rollable display, a foldable display, a stretchable display, a laser printer, a telephone, a mobile phone, a tablet personal computer, a phablet, a personal digital assistant (PDA), a wearable device, a laptop computer, a digital camera, a camcorder, a viewfinder, a micro display, a 3D display, a virtual reality display, an augmented reality display, a vehicle, a video wall including multiple displays tiled together, a theater screen, a stadium screen, a phototherapy device, or a signboard.

Because the light-emitting device has effects of improved driving voltage, excellent or suitable luminescence efficiency, and long lifespan, the electronic equipment including the light-emitting device may have characteristics with high luminance, high resolution, and low power consumption.

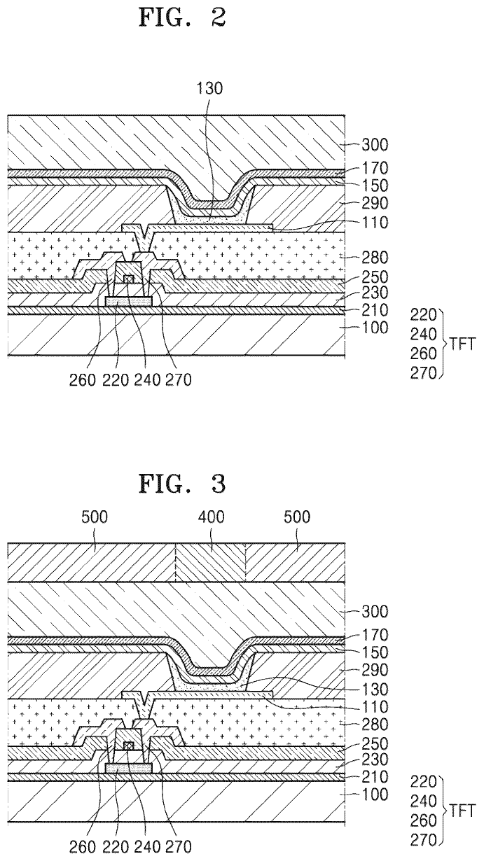

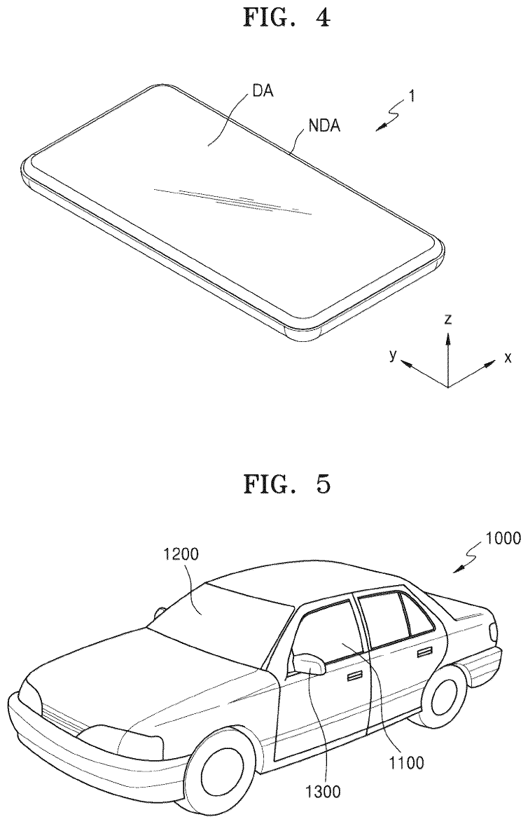

### Description of FIGS. 2 and 3

FIG. 2 is a cross-sectional view of an electronic apparatus according to one or more embodiments of the present disclosure.

The electronic apparatus of FIG. 2 may include a substrate 100, a thin-film transistor TFT, a light-emitting device, and an encapsulation portion 300.

The substrate 100 may be a flexible substrate, a glass substrate, or a metal substrate. A buffer layer 210 may be on the substrate 100. The buffer layer 210 may prevent or reduce penetration of impurities through the substrate 100, and provide a flat surface on the substrate 100.

The thin-film transistor TFT may be on the buffer layer 210. The thin-film transistor TFT may include an activation layer 220, a gate electrode 240, a source electrode 260, and a drain electrode 270.

The activation layer 220 may include an inorganic semiconductor, such as silicon or polysilicon, an organic semiconductor, or an oxide semiconductor, and may include a source region, a drain region, and a channel region.

A gate insulating film 230 for insulating the activation layer 220 from the gate electrode 240 may be on the activation layer 220, and the gate electrode 240 may be on the gate insulating film 230.

An interlayer insulating film 250 may be on the gate electrode 240. The interlayer insulating film 250 may be arranged between the gate electrode 240 and the source electrode 260 and between the gate electrode 240 and the drain electrode 270, to insulate these electrodes from one another.

The source electrode 260 and the drain electrode 270 may be on the interlayer insulating film 250. The interlayer insulating film 250 and the gate insulating film 230 may be formed to expose the source region and the drain region of the activation layer 220, and the source electrode 260 and the drain electrode 270 may be arranged in contact with the exposed portions of the source region and the drain region of the activation layer 220, respectively.

The thin-film transistor TFT may be electrically connected to the light-emitting device to drive the light-emitting device, and may be covered and protected by a passivation layer 280. The passivation layer 280 may include an inorganic insulating film, an organic insulating film, or any combination thereof. The light-emitting device may be provided on the passivation layer 280. The light-emitting device includes a first electrode 110, an interlayer, and a second electrode 150.

The first electrode 110 may be on the passivation layer 280. The passivation layer 280 may be arranged to expose a portion of the drain electrode 270 without fully covering the drain electrode 270, and the first electrode 110 may be arranged to be connected to the exposed portion of the drain electrode 270.

A pixel defining layer 290 including an insulating material may be on the first electrode 110. The pixel defining layer 290 may expose a certain region of the first electrode 110, and the interlayer may be formed in the exposed region of the first electrode 110. The pixel defining layer 290 may be a polyimide-based organic film or a polyacrylic-based organic film. In one or more embodiments, at least some layers of the interlayer may extend beyond the upper portion of the pixel defining layer 290 to be arranged in the form of a common layer. For example, at least some layers of the interlayer may extend beyond the top of the pixel-defining film 290, forming a common layer.

The second electrode 150 may be on the interlayer, and a capping layer 170 may be additionally formed on the second electrode 150. The capping layer 170 may be formed to cover the second electrode 150.

The encapsulation portion 300 may be located on the capping layer 170. The encapsulation portion 300 may be arranged on the light-emitting device to protect the light-emitting device from moisture and/or oxygen. The encapsulation portion 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic-based resin (e.g., polymethyl methacrylate, polyacrylic acid, and/or the like), an epoxy-based resin (e.g., aliphatic glycidyl ether (AG E), and/or the like), or any combination thereof; or any combination of the inorganic film(s) and the organic film(s).

FIG. 3 is a cross-sectional view of an electronic apparatus according to one or more embodiments of the present disclosure.

The electronic apparatus of FIG. 3 is substantially the same as the electronic apparatus of FIG. 2, except that a light-shielding pattern 500 and a functional region 400 are additionally arranged on the encapsulation portion 300. The functional region 400 may be i) a color filter area, ii) a color conversion area, or iii) a combination of the color filter area and the color conversion area. In one or more embodiments, the light-emitting device included in the electronic apparatus of FIG. 3 may be a tandem light-emitting device.

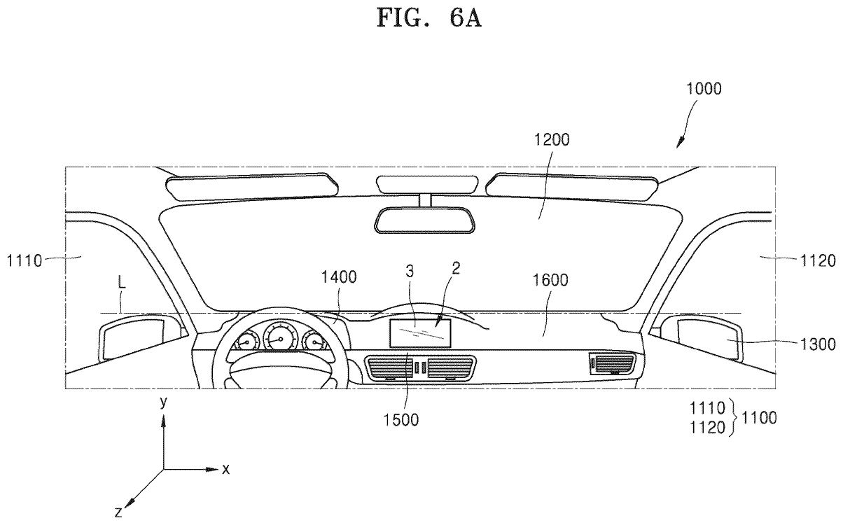

### Description of FIG. 4

FIG. 4 is a schematic perspective view of electronic equipment 1 including a light-emitting device, according to one or more embodiments of the present disclosure. The electronic equipment 1 may be, as an electronic apparatus that displays a moving image or a still image, a portable electronic equipment, such as a mobile phone, a smart phone, a tablet personal computer (PC), a mobile communication terminal, an electronic notebook, an electronic book, a portable multimedia player (PMP), a navigation, or a ultra-mobile PC (UMPC), as well as one or more suitable products or a part thereof, such as a television, a laptop, a monitor, a billboard, or an Internet of things (IoT) device. In one or more embodiments, the electronic equipment 1 may be a wearable device or a part thereof, such as a smart watch, a watch phone, a glasses-type or kind display, or a head mounted display (HMD). However, embodiments of the present disclosure are not limited thereto. For example, in one or more embodiments, the electron equipment 1 may include a dashboard of a vehicle, a center information display on a center fascia or dashboard of a vehicle, a room mirror display replacing a side mirror of a vehicle, an entertainment display arranged for the rear seat of a vehicle or arranged on the back of the front seat thereof, a head-up display (HUD) installed at the front of a vehicle or projected on a front window glass thereof, or a computer generated hologram augmented reality head up display (CGH AR HUD). FIG. 4 illustrates one or more embodiments in which the electronic equipment 1 is a smart phone for convenience of description.

The electronic equipment 1 may include a display area DA and a non-display area NDA outside the display area DA. The electronic equipment 1 may implement an image through an array of a plurality of pixels that are two-dimensionally arranged in the display area DA.

The non-display area NDA is an area that does not display an image, and may entirely be around (e.g., surround) the display area DA. On the non-display area NDA, a driver for providing electrical signals or power to display devices arranged on the display area DA may be arranged. On the non-display area NDA, a pad, which is an area to which an electronic element or a printed circuit board, may be electrically connected may be arranged.

In the electronic equipment 1, a length in an x-axis direction and a length (e.g., a width) in a y-axis direction may be different from each other. In one or more embodiments, as shown in FIG. 4, the length in the x-axis direction may be shorter than the length (e.g., the width) in the y-axis direction. In one or more embodiments, the length in the x-axis direction may be substantially the same as the length (e.g., the width) in the y-axis direction. In one or more embodiments, the length in the x-axis direction may be greater than the length (e.g., the width) in the y-axis direction.

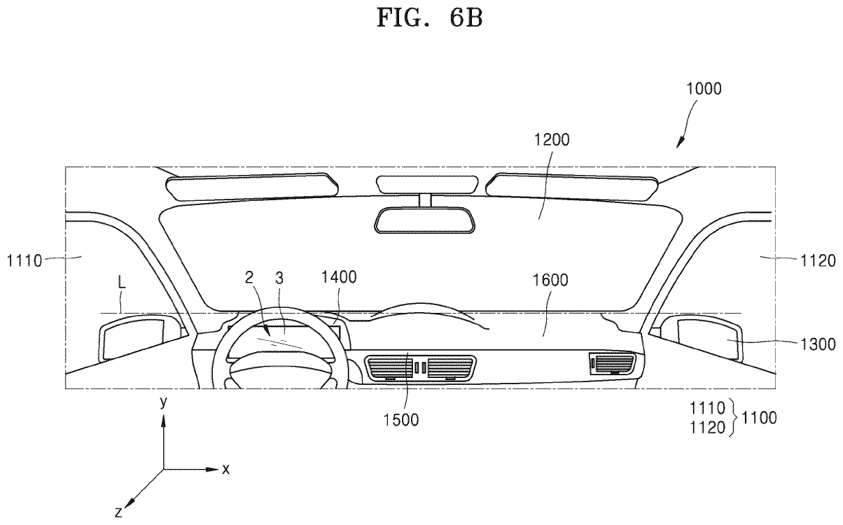

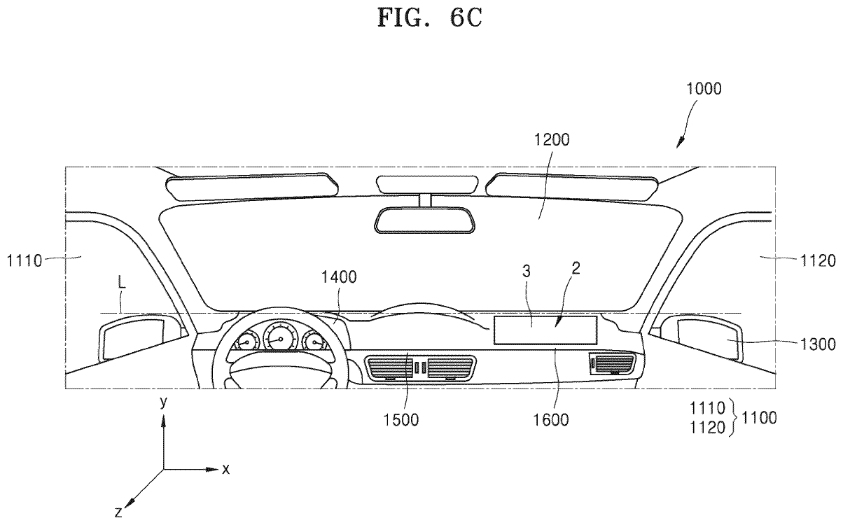

### Descriptions of FIGS. 5 and 6A to 6C

FIG. 5 is a schematic view of an exterior of a vehicle 1000 as electronic equipment including the light-emitting device according to one or more embodiments. FIGS. 6A to 6C are each a schematic view of an interior of the vehicle 1000 according to one or more embodiments.

Referring to FIGS. 5, 6A, 6B, and 6C, the vehicle 1000 may refer to one or more suitable apparatuses for moving an object to be transported, such as a human, an object, or an animal, from a departure point to a destination point. The vehicle 1000 may include a vehicle traveling on a road or a track, a vessel moving over the sea or a river, an airplane flying in the sky using the action of air, and/or the like.

In one or more embodiments, the vehicle 1000 may travel on a road or a track. The vehicle 1000 may move in a certain direction according to rotation of at least one wheel thereof. In one or more embodiments, the vehicle 1000 may include a three-wheeled or four-wheeled vehicle, a construction machine, a two-wheeled vehicle, a prime mover device, a bicycle, or a train running on a track.

The vehicle 1000 may include a body having an interior and an exterior, and a chassis in which mechanical apparatuses necessary for driving are installed as other parts except for the body of the vehicle 1000. The exterior of the body may include a front panel, a bonnet, a roof panel, a rear panel, a trunk, a pillar provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side-view mirror 1300, a cluster 1400, a center fascia 1500, a passenger seat dashboard 1600, and a display apparatus 2.

The side window glass 1100 and the front window glass 1200 may be partitioned by a pillar arranged between the side window glass 1100 and the front window glass 1200.

The side window glass 1100 may be installed on a side of the vehicle 1000. In one or more embodiments, the side window glass 1100 may be installed on a door of the vehicle 1000. A plurality of side window glasses 1100 may be provided and may face each other. In one or more embodiments, the side window glass 1100 may include a first side window glass 1110 and a second side window glass 1120. In one or more embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced and/or apart (e.g., spaced apart or separated) from each other in an x direction or a −x direction (the direction opposite the x-direction). In one or more embodiments, the first side window glass 1110 and the second side window glass 1120 may be spaced and/or apart (e.g., spaced apart or separated) from each other in the x direction or the −x direction. For example, an imaginary straight line L connecting the side window glasses 1100 may extend in the x direction or the −x direction. In one or more embodiments, an imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 opposite to (e.g., facing) each other.

The side-view mirror 1300 may provide a rear view of the vehicle 1000. The side-view mirror 1300 may be installed on the exterior of the body of the vehicle 1000. In one or more embodiments, a plurality of side-view mirrors 1300 may be provided. Any one of the plurality of side-view mirrors 1300 may be arranged outside the first side window glass 1110. The other one of the plurality of side-view mirrors 1300 may be arranged outside the second side window glass 1120.

The cluster 1400 may be arranged in front of a steering wheel. The cluster 1400 may include a tachometer, a speedometer, a coolant thermometer, a fuel gauge, a turn signal indicator, a high beam indicator, a warning light, a seat belt warning light, an odometer, a tachograph, an automatic shift selector indicator, a door open warning light, an engine oil warning light, and/or a low fuel warning light.

The center fascia 1500 may include a control panel on which a plurality of buttons for adjusting an audio device, an air conditioning device, and/or a seat heater are arranged. The center fascia 1500 may be arranged on one side of the cluster 1400.

The passenger seat dashboard 1600 may be spaced and/or apart (e.g., spaced apart or separated) from the cluster 1400, and the center fascia 1500 may be arranged between the cluster 1400 and the passenger seat dashboard 1600. In one or more embodiments, the cluster 1400 may be arranged to correspond to a driver seat, and the passenger seat dashboard 1600 may be arranged to correspond to a passenger seat. In one or more embodiments, the cluster 1400 may be adjacent to the first side window glass 1110, and the passenger seat dashboard 1600 may be adjacent to the second side window glass 1120.

In one or more embodiments, the display apparatus 2 may include a display panel 3, and the display panel 3 may display an image. The display apparatus 2 may be arranged inside the vehicle 1000. In one or more embodiments, the display apparatus 2 may be arranged between the side window glasses 1100 opposite to (e.g., facing) each other. The display apparatus 2 may be arranged on at least one of the cluster 1400, the center fascia 1500, or the passenger seat dashboard 1600.

The display device 2 may include an organic light-emitting display device, an inorganic light-emitting display device, a quantum dot display device, and/or the like. Hereinafter, as the display device 2 according to one or more embodiments, an organic light-emitting display apparatus including the aforementioned light-emitting device according to one or more embodiments will be described as an example, but one or more suitable types (kinds) of the aforementioned display apparatus may be used in embodiments.

Referring to FIG. 6A, in one or more embodiments, the display apparatus 2 may be arranged on the center fascia 1500. In one or more embodiments, the display apparatus 2 may display navigation information. In one or more embodiments, the display apparatus 2 may display information regarding audio settings, video setting, or vehicle settings.

Referring to FIG. 6B, in one or more embodiments, the display apparatus 2 may be arranged on the cluster 1400. In these embodiments, the cluster 1400 may display driving information and/or the like through the display apparatus 2. For example, the cluster 1400 may digitally implement driving information and/or the like. The cluster 1400 may digitally implement vehicle information and driving information as images. In one or more embodiments, a needle and a gauge of a tachometer and one or more suitable warning light icons may be displayed by a digital signal.

Referring to FIG. 6C, in one or more embodiments, the display device 2 may be arranged on the passenger seat dashboard 1600. The display apparatus 2 may be embedded in the passenger seat dashboard 1600 or arranged on the passenger seat dashboard 1600. In one or more embodiments, the display device 2 arranged on the passenger seat dashboard 1600 may display an image related to information displayed on the cluster 1400 and/or information displayed on the center fascia 1500. In one or more embodiments, the display apparatus 2 arranged on the passenger seat dashboard 1600 may display information different from information displayed on the cluster 1400 and/or information displayed on the center fascia 1500.

### Manufacturing Method

The layers constituting the hole transport region 120, the emission layer 130, and the layers constituting the electron transport region 140 may be formed in a certain region by using one or more suitable methods such as vacuum deposition, spin coating, casting, Langmuir-Blodgett (LB) deposition, ink-jet printing, laser-printing, laser-induced thermal imaging, and/or the like.

When the layers constituting the hole transport region 120, the emission layer 130, and the layers constituting the electron transport region 140 are each formed by vacuum deposition, the deposition may be performed at a deposition temperature of about 100° C. to about 500° C., a vacuum degree of about 10-8 torr to about 10-3 torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as used herein refers to a cyclic group including (e.g., consisting of) carbon atoms as the only ring-forming atoms and having 3 to 60 carbon atoms.

The term “C₁-C₆₀ heterocyclic group” as used herein refers to a cyclic group that has 1 to 60 carbon atoms and further has, in addition to carbon atoms, a heteroatom as a ring-forming atom.

The C₃-C₆₀ carbocyclic group and the C₁-C₆₀ heterocyclic group may each be: a monocyclic group including (e.g., consisting of) one (e.g., exactly one) ring; or a polycyclic group in which two or more rings are condensed with each other. For example, the number of ring-forming atoms of the C₁-C₆₀ heterocyclic group may be from 3 to 61.

The term “cyclic group” as used herein may include both (e.g., simultaneously) the C₃-C₆₀ carbocyclic group and the C₁-C₆₀ heterocyclic group.

The term “T electron-rich C₃-C₆₀ cyclic group” as used herein refers to a cyclic group that has 3 to 60 carbon atoms and does not include *—N═*′ as a ring-forming moiety.

The term “T electron-deficient nitrogen-containing C₁-C₆₀ cyclic group” as used herein refers to a heterocyclic group that has 1 to 60 carbon atoms and includes *—N═*′ as a ring-forming moiety.

In one or more embodiments,
the C₃-C₆₀ carbocyclic group may be i) Group T1 or ii) a condensed cyclic group in which two or more of Group T1 are condensed with each other (e.g., a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) Group T2, ii) a condensed cyclic group in which two or more of Group T2 are condensed with each other, or iii) a condensed cyclic group in which at least one Group T2 and at least one Group T1 are condensed with each other (e.g., a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, a xanthene group, and/or the like),
the π electron-rich C₃-C₆₀ cyclic group may be i) Group T1, ii) a condensed cyclic group in which two or more of Group T1 are condensed with each other, iii) Group T3, iv) a condensed cyclic group in which two or more of Group T3 are condensed with each other, or v) a condensed cyclic group in which at least one Group T₃ and at least one Group T1 are condensed with each other (e.g., the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like), and
the π electron-deficient nitrogen-containing C₁-C₆₀ cyclic group may be i) Group T4, ii) a condensed cyclic group in which two or more Groups T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T4, at least one Group T1, and at least one Group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like).

Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group.

Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group.

Group T3 may be a furan group, a thiophene group, a 1H-pyrrole group, a silole group, or a borole group.

Group T4 may include a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group,” “the C₃-C₆₀ carbocyclic group,” “the C₁-C₆₀ heterocyclic group,” “the T electron-rich C₃-C₆₀ cyclic group,” or “the T electron-deficient nitrogen-containing C₁-C₆₀ cyclic group” as used herein refer to i) a group condensed to any cyclic group, ii) a monovalent group, or iii) a polyvalent group (e.g., a divalent group, a trivalent group, a tetravalent group, and/or the like), according to the structure of a formula for which the corresponding term is used.

In one or more embodiments, the “benzene group” may be a benzo group, a phenyl group, a phenylene group, and/or the like, which may be easily understood by those of ordinary skill in the art according to the structure of a formula including the “benzene group.”

Non-limiting examples of the monovalent C₃-C₆₀ carbocyclic group and monovalent C₁-C₆₀ heterocyclic group are a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Non-limiting examples of the divalent C₃-C₆₀ carbocyclic group and the divalent C₁-C₆₀ heterocyclic group are a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group that has 1 to 60 carbon atoms, and non-limiting examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group.

The term “C₁-C₆₀ alkylene group” as used herein refers to a divalent group having substantially the same structure as the C₁-C₆₀ alkyl group.

The term “C₂-C₆₀ alkenyl group” as used herein refers to a monovalent hydrocarbon group having at least one carbon-carbon double bond in the middle or at the terminus of a C₂-C₆₀ alkyl group, and non-limiting examples thereof may include an ethenyl group, a propenyl group, a butenyl group, and/or the like.

The term “C₂-C₆₀ alkenylene group” as used herein refers to a divalent group having substantially the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of a C₂-C₆₀ alkyl group, and non-limiting examples thereof may include an ethynyl group, a propynyl group, and/or the like.

The term “C₂-C₆₀ alkynylene group” as used herein refers to a divalent group having substantially the same structure as the C₂-C₆₀ alkynyl group.

The term “C₁-C₆₀ alkoxy group” as used herein refers to a monovalent group represented by —OA₁₀₁ (wherein A₁₀₁ is a C₁-C₆₀ alkyl group), and non-limiting examples thereof may include a methoxy group, an ethoxy group, an isopropyloxy group, and/or the like.

The term “C₃-C₁₀ cycloalkyl group” as used herein refers to a monovalent saturated hydrocarbon cyclic group having 3 to 10 carbon atoms, and non-limiting examples thereof may include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, a bicyclo[2.2.2]octyl group, and/or the like.

The term “C₃-C₁₀ cycloalkylene group” as used herein refers to a divalent group having substantially the same structure as the C₃-C₁₀ cycloalkyl group.

The term “C₁-C₁₀ heterocycloalkyl group” as used herein refers to a monovalent cyclic group of 1 to 10 carbon atoms, further including, in addition to carbon atoms, at least one heteroatom as ring-forming atoms, and non-limiting examples thereof may include a 1,2,3,4-oxatriazolidinyl group, a tetrahydrofuranyl group, a tetrahydrothiophenyl group, and/or the like.

The term “C₁-C₁₀ heterocycloalkylene group” as used herein refers to a divalent group having substantially the same structure as the C₁-C₁₀ heterocycloalkyl group.

The term “C₃-C₁₀ cycloalkenyl group” as used herein refers to a monovalent cyclic group that has 3 to 10 carbon atoms, at least one carbon-carbon double bond in the ring thereof, and no aromaticity, and non-limiting examples thereof may include a cyclopentenyl group, a cyclohexenyl group, a cycloheptenyl group, and/or the like.

The term “C₃-C₁₀ cycloalkenylene group” as used herein refers to a divalent group having substantially the same structure as the C₃-C₁₀ cycloalkenyl group.

The term “C₁-C₁₀ heterocycloalkenyl group” as used herein refers to a monovalent cyclic group of 1 to 10 carbon atoms, further including, in addition to carbon atoms, at least one heteroatom as ring-forming atoms and at least one double bond in the cyclic structure thereof. Non-limiting examples of the C₁-C₁₀ heterocycloalkenyl group may include a 4,5-dihydro-1,2,3,4-oxatriazolyl group, a 2,3-dihydrofuranyl group, a 2,3-dihydrothiophenyl group, and/or the like.

The term “C₁-C₁₀ heterocycloalkylene group” as used herein refers to a divalent group having substantially the same structure as the C₁-C₁₀ heterocycloalkyl group.

The term “C₆-C₆₀ aryl group” as used herein refers to a monovalent group having a carbocyclic aromatic system of 6 to 60 carbon atoms.

The term “C₆-C₆₀ arylene group” as used herein refers to a divalent group having a carbocyclic aromatic system of 6 to 60 carbon atoms.

Non-limiting examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and/or the like.

When the C₆-C₆₀ aryl group and the C₆-C₆₀ arylene group each include two or more rings, the two or more rings may be condensed with each other.

The term “C₁-C₆₀ heteroaryl group” as used herein refers to a monovalent group having a heterocyclic aromatic system of 1 to 60 carbon atoms, further including, in addition to carbon atoms, at least one heteroatom, as ring-forming atoms.

The term “C₁-C₆₀ heteroarylene group” as used herein refers to a divalent group having a heterocyclic aromatic system of 1 to 60 carbon atoms, further including, in addition to carbon atoms, at least one heteroatom, as ring-forming atoms.

Non-limiting examples of the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and/or the like.

When the C₁-C₆₀ heteroaryl group and the C₁-C₆₀ heteroarylene group each include two or more rings, the two or more rings may be condensed with each other.

The term “monovalent non-aromatic condensed polycyclic group” as used herein refers to a monovalent group (e.g., having 8 to 60 carbon atoms) having two or more rings condensed to each other, only carbon atoms as ring-forming atoms, and no aromaticity in the entire molecular structure as a whole. Non-limiting examples of the monovalent non-aromatic condensed polycyclic group may include an indenyl group, a fluorenyl group, a spiro-bifluorenyl group, a benzofluorenyl group, an indenophenanthrenyl group, an indeno anthracenyl group, and/or the like.

The term “divalent non-aromatic condensed polycyclic group” as used herein refers to a divalent group having substantially the same structure as the monovalent non-aromatic condensed polycyclic group described above.

The term “monovalent non-aromatic condensed heteropolycyclic group” as used herein refers to a monovalent group (e.g., having 1 to 60 carbon atoms) having two or more rings condensed to each other, further including, in addition to carbon atoms, at least one heteroatom, as ring-forming atoms, and having no aromaticity in its entire molecular structure as a whole. Non-limiting examples of the monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group.

The term “divalent non-aromatic condensed heteropolycyclic group” as used herein refers to a divalent group having substantially the same structure as the monovalent non-aromatic condensed heteropolycyclic group.

The term “C₆-C₆₀ aryloxy group” as used herein indicates —OA₁₀₂ (wherein A₁₀₂ is a C₆-C₆₀ aryl group).

The term “C₆-C₆₀ arylthio group” as used herein indicates —SA₁₀₃ (wherein A₁₀₃ is a C₆-C₆₀ aryl group).

The term “C₇-C₆₀ arylalkyl group” as used herein refers to -A₁₀₄A₁₀₅ (wherein A₁₀₄ is a C₁-C₅₄ alkylene group, and A₁₀₅ is a C₆-C₅₉ aryl group).

The term “C₂-C₆₀ heteroarylalkyl group” as used herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as used herein may be:
deuterium, tritium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, tritium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁n)(Q₁₂)(Q₁₃), —N(Q₁n)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, tritium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

In the present disclosure, Q₁ to Q₃, Qu to Q₁₃, Q₂₁ to Q₂₃ and Q₃₁ to Q₃₃ may each independently be: hydrogen; deuterium; tritium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, tritium, —F, a cyano group, a C₁-C₆₀ alkyl group, a C₁-C₆₀ alkoxy group, a phenyl group, a biphenyl group, or any combination thereof.

The term “heteroatom” as used herein refers to any atom other than a carbon atom. Non-limiting examples of the heteroatom may include O, S, N, P, Si, B, Ge, Se, and any combination thereof.

The term “transition metal” as used herein may include Hf, Ta, W, Re, Os, Ir, Pt, Au, and/or the like.

In the disclosure, “D” may refer to deuterium, “T” may refer to tritium, “Ph” may refer to a phenyl group, “Me” may refer to a methyl group, “Et” may refer to an ethyl group, “tert-Bu”, “^{t}Bu”, or “Bu^{t}” may refer to a tert-butyl group, and “0 Me” may refer to a methoxy group.

The term “biphenyl group” as used herein refers to “a phenyl group that is substituted with a phenyl group.” For example, the “biphenyl group” may be a substituted phenyl group having a C₆-C₆₀ aryl group as a substituent.

The term “terphenyl group” as used herein refers to “a phenyl group substituted with a biphenyl group.” The “terphenyl group” may belong to i) “a substituent phenyl group” which is “a C₆-C₆₀ aryl group in which a substituent is substituted with a C₆-C₆₀ aryl group”, or ii) “a substituted phenyl group” having two substituents, each of which is “a C₆-C₆₀ aryl group.”

* and *′ as used herein, unless defined otherwise, each refer to a binding site to a neighboring atom in a corresponding formula or moiety.

In the disclosure, the x-axis, y-axis, and z-axis are not limited to three axes in an orthogonal coordinate system, and may be interpreted in a broad sense including these axes. For example, the x-axis, y-axis, and z-axis may describe axes that are orthogonal to each other, or may describe axes that are in different directions that are not orthogonal to each other.

Hereinafter, organic compounds according to one or more embodiments and light-emitting devices according to one or more embodiments will be described in more detail with reference to the following Synthesis Examples and Examples. The wording “B was used instead of A” used in describing Synthesis Examples refers to that an substantially identical molar equivalent of B was used in place of an substantially identical molar equivalent of A.

### Synthesis Example 1 (Synthesis of Compound BH1)

In an argon atmosphere, 10-phenyl-9-anthraceneboronic acid (2.98 g, 10.0 mmol), 5-bromo-1,2,3-triphenyl-1H-indole (4.24 g, 10.0 mmol), tetrakis(triphenylphosphine)dipalladium(0) (0.23 g, 0.20 mmol), and 1,2-dimethoxyethane solution (100 mL) containing 2 M sodium carbonate aqueous solution (15 mL, 30.0 mmol) were stirred under heating and reflux (e.g., the reaction mixture was heated while vapors were condensed back into the liquid and returned to a flask, maintaining a constant temperature) for 4 hours. Toluene and water were added to the reaction solution, and an organic layer was separated therefrom and purified by column chromatography to obtain Compound BH1 (4.30 g, yield of 72%). Field Desorption Mass Spectroscopy (FD-MS): m/z=597 (MW: 597.76).

### Synthesis Example 2 (synthesis of Compound BH5)

Compound BH5 was synthesized in substantially the same manner as in the synthesis of Compound BH1, except that 5-bromo-1,3-diphenyl-1H-indole was used instead of 5-bromo-1,2,3-triphenyl-1H-indole. FD-MS: m/z=521 (MW: 521.66).

### Synthesis Example 3 (synthesis of Compound BH6)

Compound BH6 was synthesized in substantially the same manner as in the synthesis of Compound BH1, except that 5-bromo-1,2-diphenyl-1H-indole was used instead of 5-bromo-1,2,3-triphenyl-1H-indole. FD-MS: m/z=521 (MW: 521.66).

### Synthesis Example 4 (synthesis of Compound BH15)

Compound BH15 was synthesized in substantially the same manner as in the synthesis of Compound BH1, except that 10-(1,1′-biphenyl)-3-yl-9-anthraceneboronic acid was used instead of 10-phenyl-9-anthraceneboronic acid. FD-MS: m/z=673 (MW: 673.86).

### Synthesis Example 5 (Synthesis of Compound BH43)

Compound BH43 was synthesized in substantially the same manner as in the synthesis of Compound BH1, except that 10-(phenyl-2,3,4,5,6-D₅)-9-anthraceneboronic acid was used instead of 10-phenyl-9-anthraceneboronic acid. FD-MS: m/z=602 (MW: 602.79).

### Synthesis Example 6 (Synthesis of Compound BH49)

Compound BH49 was synthesized in substantially the same manner as in the synthesis of Compound BH1, except that 10-(phenyl-2,3,4,5,6-D₅)-9-anthraceneboronic acid was used instead of 10-phenyl-9-anthraceneboronic acid and 5-bromo-1,2-diphenyl-1H-indole was used instead of 5-bromo-1,2,3-triphenyl-1H-indole. FD-MS: m/z=526 (MW: 526.69).

### Comparative Example 1

As an anode, an ITO-patterned glass substrate was cut to a size of 50 mm×50 mm×0.5 mm, sonicated with isopropyl alcohol and (then with) distilled water, each for 10 minutes, and then cleaned by irradiation with UV and exposure to ozone for 10 minutes. The resulting ITO patterned glass substrate was loaded onto a vacuum deposition apparatus.

On the anode, Compound HH1 and Compound HI were vacuum-deposited at a weight ratio of 10:3 to form a hole injection layer having a thickness of 10 nm. On the hole injection layer, Compound HH1 was vacuum-deposited to form a hole transport layer having a thickness of 85 nm. On the hole transport layer, Compound HH2 was vacuum-deposited to form an electron blocking layer having a thickness of 5 nm.

On the electron blocking layer, Compound CH1 as a host and Compound BD as a dopant were vacuum-deposited to form an emission layer having a thickness of 20 nm. Here, a weight ratio of the host to the dopant was 20:1.

On the emission layer, Compound EE1 was vacuum-deposited to form a hole blocking layer having a thickness of 5 nm. On the hole blocking layer, Compound EE2 and LiQ were vacuum-deposited at a weight ratio of 31:50 to form an electron transport layer having a thickness of 31 nm. On the electron transport layer, LiQ was vacuum-deposited to form an electron injection layer having a thickness of 1 nm, and then, Al was vacuum-deposited thereon to form a cathode having a thickness of 80 nm, thereby completing manufacture of a light-emitting device.

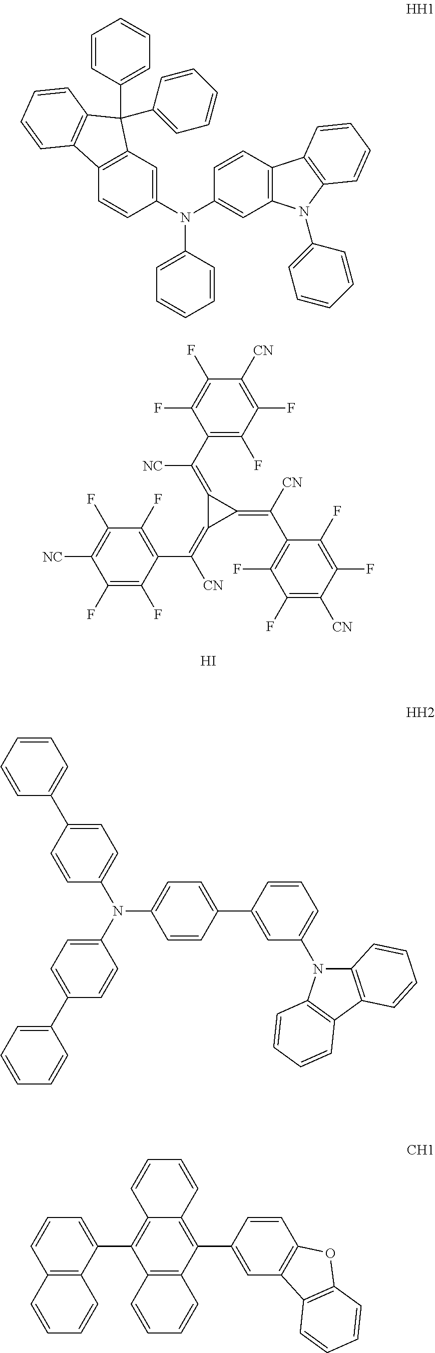

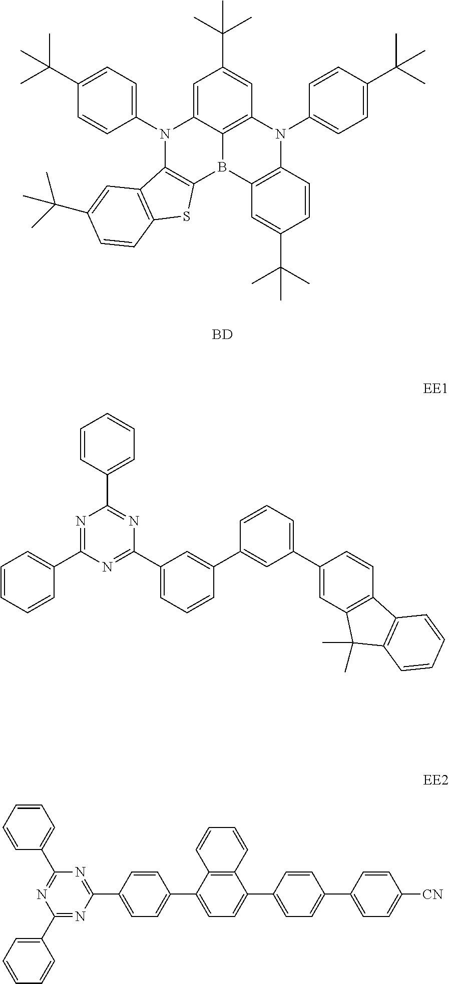 [SMILES: CC(C)(C)c1ccc(N2c3ccc(C(C)(C)C)cc3B3c4sc5ccc(C(C)(C)C)cc5c4N(c4ccc(C(C)(C)C)cc4)c4cc(C(C)(C)C)cc2c43)cc1 ; "BD" ; CC1(C)c2ccccc2-c2ccc(-c3cccc(-c4cccc(-c5nc(-c6ccccc6)nc(-c6ccccc6)n5)c4)c3)cc21 ; N#Cc1ccc(-c2ccc(-c3ccc(-c4ccc(-c5nc(-c6ccccc6)nc(-c6ccccc6)n5)cc4)c4ccccc34)cc2)cc1]

### Comparative Examples 2 to 9 and Examples 1 to 6

Light-emitting devices were each manufactured in substantially the same manner as in Comparative Example 1, except that different types (kinds) of a host as shown in Table 1 were respectively used in forming an emission layer.

### Examples 7 to 18

Light-emitting devices were each manufactured in substantially the same manner as in Comparative Example 1, except that a mixture of hosts as shown in Table 2 was used instead of a single host in forming an emission layer. The parentheses shown in Table 2 indicate the weight ratio of two compounds used for the mixture of hosts.

### Evaluation Example 1

For each of the light-emitting devices of Comparative Examples 1 to 9 and Examples 1 to 18, the external quantum efficiency (EQE) was measured, and results are shown in Tables 1 and 2.

In evaluating the EQE, the luminance/current density was measured by using a luminance meter that was calibrated for wavelength sensitivity, and the EQ E was measured by assuming an angular luminance distribution (Lambertian) which introduced a perfect reflecting diffuser. Based on the measurement of Comparative Example 1 as a standard (100%), the EQE values of Comparative Examples 2 to 9 and Examples 1 to 18 are shown in Tables 1 and 2.

TABLE 1

| No. | host | EQE @ 10 mA/cm² |
|---|---|---|
| Comparative Example 1 | CH1 | 100% |
| Comparative Example 2 | CH2 | 95% |
| Comparative Example 3 | CH3 | 87% |
| Comparative Example 4 | CE1 | 72% |
| Comparative Example 5 | CE2 | 88% |
| Comparative Example 6 | CE3 | 49% |
| Comparative Example 7 | CE4 | 95% |
| Comparative Example 8 | CE5 | 90% |
| Comparative Example 9 | CE6 | 77% |
| Example 1 | BH1 | 113% |
| Example 2 | BH5 | 105% |
| Example 3 | BH6 | 118% |
| Example 4 | BH15 | 111% |
| Example 5 | BH43 | 113% |
| Example 6 | BH49 | 118% |

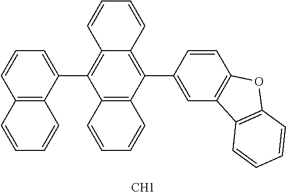

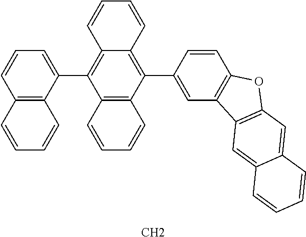

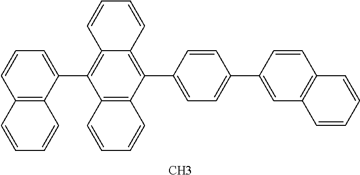

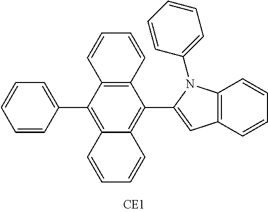 [SMILES: c1ccc(-c2c3ccccc3c(-c3cc4ccccc4n3-c3ccccc3)c3ccccc23)cc1]

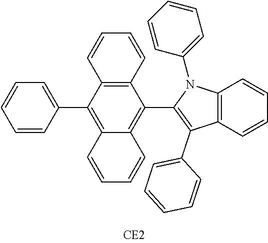 [SMILES: c1ccc(-c2c3ccccc3c(-c3c(-c4ccccc4)c4ccccc4n3-c3ccccc3)c3ccccc23)cc1]

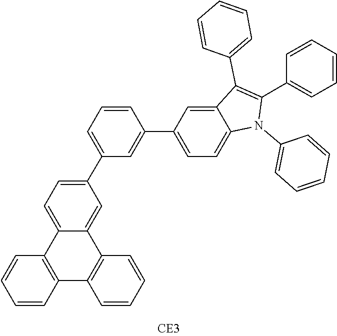 [SMILES: c1ccc(-c2c(-c3ccccc3)n(-c3ccccc3)c3ccc(-c4cccc(-c5ccc6c7ccccc7c7ccccc7c6c5)c4)cc23)cc1]

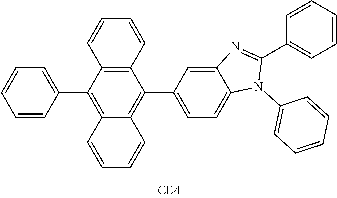 [SMILES: c1ccc(-c2c3ccccc3c(-c3ccc4c(c3)nc(-c3ccccc3)n4-c3ccccc3)c3ccccc23)cc1]

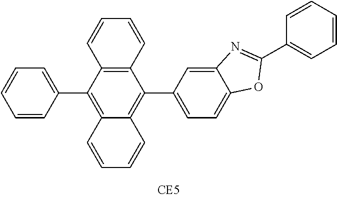 [SMILES: c1ccc(-c2nc3cc(-c4c5ccccc5c(-c5ccccc5)c5ccccc45)ccc3o2)cc1]

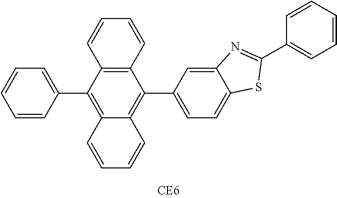 [SMILES: c1ccc(-c2nc3cc(-c4c5ccccc5c(-c5ccccc5)c5ccccc45)ccc3s2)cc1]

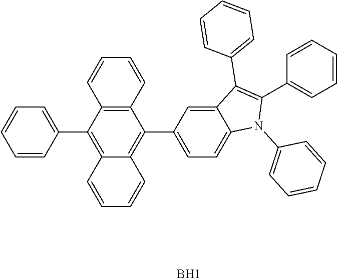

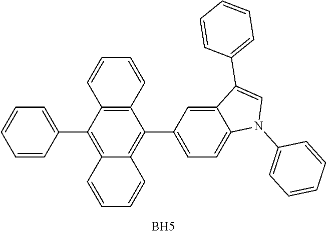

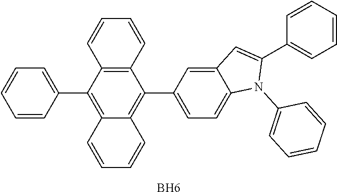

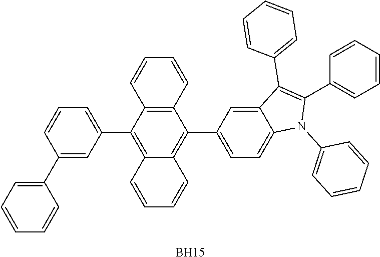 [SMILES: c1ccc(-c2cccc(-c3c4ccccc4c(-c4ccc5c(c4)c(-c4ccccc4)c(-c4ccccc4)n5-c4ccccc4)c4ccccc34)c2)cc1 ; "BH15"]

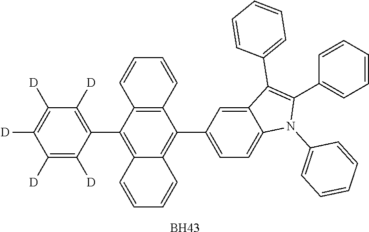

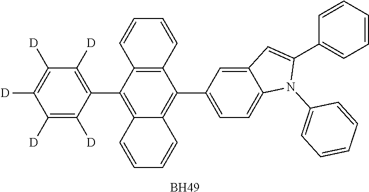

Referring to Table 1, it was confirmed that each of the light-emitting devices of Examples 1 to 6 including the organic compound of Formula 1 had high EQE compared to the light-emitting devices of Comparative Examples 1 to 9.

TABLE 2

| No. | Host | EQE @10 mA/cm² |
|---|---|---|
| Comparative | CH1 | 100% |
| Example 1 | | |
| Comparative | CH2 | 95% |
| Example 2 | | |
| Comparative | CH3 | 87% |
| Example 3 | | |
| Example 1 | BH1 | 113% |
| Example 5 | BH43 | 113% |
| Example 7 | CH1:BH1 (1:1) | 118% |
| Example 8 | CH1:BH1 (1:3) | 119% |
| Example 9 | CH2:BH1 (2:1) | 114% |
| Example 10 | CH2:BH1 (1:1) | 119% |
| Example 11 | CH2:BH1 (1:2) | 120% |
| Example 12 | CH3:BH1 (1:3) | 115% |
| Example 13 | CH1:BH43 (1:1) | 118% |
| Example 14 | CH1:BH43 (1:3) | 119% |
| Example 15 | CH2:BH43 (2:1) | 114% |
| Example 16 | CH2:BH43 (1:1) | 119% |
| Example 17 | CH2:BH43 (1:2) | 120% |
| Example 18 | CH3:BH43 (1:3) | 115% |

Referring to Table 2, it was confirmed that, even if a compound not belonging to the scope of Formula 1 (i.e., not represented by Formula 1) was mixed with the organic compound of Formula 1, the light-emitting devices having the mixture of hosts each had higher EQE compared to the light-emitting device including a single host.

According to the one or more embodiments, the organic compound represented by Formula 1 may be a compound in which a carbon atom at the position 9 (or 10) of an anthracene group is linked to a carbon atom of one selected from among the positions 4 to 7 of an indole group. In this regard, the organic compound may have improved stability and improved hole-transporting ability, and a suitable energy level for use as a blue host, and may also be easily synthesized. Therefore, a light-emitting device including the organic compound may have improved external quantum efficiency. In other words, the organic compound represented by Formula 1 is a compound where a carbon atom at position 9 (or 10) of an anthracene group is linked to a carbon atom at one of the positions 4 to 7 of an indole group. This structural configuration may enhance the compound's stability and hole-transporting ability, making it suitable for use as a blue host in light-emitting devices. Additionally, the compound can be synthesized with relative ease. Consequently, a light-emitting device incorporating this organic compound may exhibit improved external quantum efficiency due to these enhanced properties.

In the present disclosure, it will be understood that the term “comprise(s)/comprising,” “include(s)/including,” or “have/has/having” specifies the presence of stated features, integers, steps, operations, elements, and/or components, but do not preclude the presence or addition of one or more other features, integers, steps, operations, elements, components, and/or groups thereof.

In the context of the present application and unless otherwise defined, the terms “use,” “using,” and “used” may be considered synonymous with the terms “utilize,” “utilizing,” and “utilized,” respectively.

Throughout the present disclosure, when a component such as a layer, a film, a region, or a plate is mentioned to be placed “on” another component, it will be understood that it may be directly on another component or that another component may be interposed therebetween. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

In the present disclosure, although the terms “first,” “second,” etc., may be utilized herein to describe one or more elements, components, regions, and/or layers, these elements, components, regions, and/or layers should not be limited by these terms. These terms are only utilized to distinguish one component from another component.

As utilized herein, the singular forms “a,” “an,” “one,” and “the” are intended to include the plural forms as well, unless the context clearly indicates otherwise. Further, the use of “may” when describing embodiments of the present disclosure refers to “one or more embodiments of the present disclosure”.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, or 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in the present disclosure is intended to include all higher numerical limitations subsumed therein. Accordingly, Applicant reserves the right to amend the disclosure, including the claims, to expressly recite any sub-range subsumed within the ranges expressly recited herein.

The light-emitting device, the display apparatus, the electronic apparatus, the electronic equipment, the manufacturing apparatus thereof, or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in one or more embodiments. While one or more embodiments have been described with reference to the drawings, it will be understood by those of ordinary skill in the art that one or more suitable changes in form and details may be made therein without departing from the spirit and scope as defined by the appended claims and equivalents thereof.

What is claimed is:

1. A light-emitting device comprising:
a first electrode;
a second electrode opposite to the first electrode; and
an interlayer between the first electrode and the second electrode and comprising an emission layer, wherein
the interlayer comprises a first organic compound represented by Formula 1: 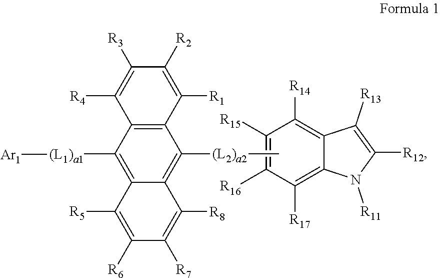
wherein, in Formula 1,
Ar₁ is a C₃-C₆₀ carbocyclic group unsubstituted or substituted with at least one R₁₀ₐ or a C₁-C₆₀ heterocyclic group unsubstituted or substituted with at least one R₁₀ₐ,
L₁ and L₂ are each independently a C₆-C₆₀ arylene group unsubstituted or substituted with at least one R₁₀ₐ or a C₁-C₆₀ heteroarylene group unsubstituted or substituted with at least one R₁₀ₐ,
a1 and a2 are each an integer from 0 to 2,
when a1 is 0, then (L₁)ₐ₁ is a single bond, and when a2 is 0, then (L₂)ₐ₂ is a single bond,
R₁ to R₈ and R₁₁ to R₁₇ are each independently hydrogen, deuterium, tritium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), provided that one selected from among R₁₄ to R₁₇ is a binding site to (L₂)ₐ₂,
R₁₀ₐ is:
deuterium, tritium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, tritium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁n)(Q₁₂)(Q₁₃), —N(Qin)(Q₁₂), —B(Qin)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, tritium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium, tritium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, tritium, —F, a cyano group, a C₁-C₆₀ alkyl group, a C₁-C₆₀ alkoxy group, a phenyl group, a biphenyl group, or any combination thereof.

2. The light-emitting device of claim 1, wherein the emission layer comprises the first organic compound.

3. The light-emitting device of claim 1, wherein the interlayer further comprises a second organic compound that is represented by Formula 2 and is different from the first organic compound: 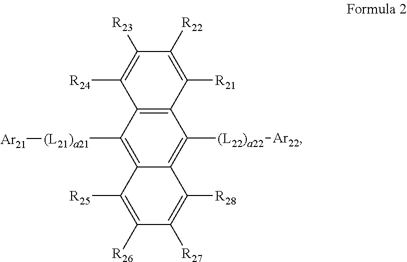
wherein, in Formula 2,
Ar₂₁ and Ar₂₂ are each independently a C₃-C₆₀ carbocyclic group unsubstituted or substituted with at least one R_{10b} or a C₁-C₆₀ heterocyclic group unsubstituted or substituted with at least one R_{10b},
L₂₁ and L₂₂ are each independently a C₆-C₆₀ arylene group unsubstituted or substituted with at least one R_{10b} or a C₁-C₆₀ heteroarylene group unsubstituted or substituted with at least one R_{10b},
a21 and a22 are each an integer from 0 to 2,
when a21 is 0, then (L₂₁)ₐ₂₁ is a single bond, and when a22 is 0, then (L₂₂)ₐ₂₂ is a single bond,
R₂₁ to R₂₈ are each independently hydrogen, deuterium, tritium, —F, —Cl, —Br, —I, a hydroxyl group, a cyano group, a nitro group, a C₁-C₆₀ alkyl group unsubstituted or substituted with at least one R_{10b}, a C₂-C₆₀ alkenyl group unsubstituted or substituted with at least one R_{10b}, a C₂-C₆₀ alkynyl group unsubstituted or substituted with at least one R_{10b}, a C₁-C₆₀ alkoxy group unsubstituted or substituted with at least one R_{10b}, a C₁-C₆₀ alkylthio group unsubstituted or substituted with at least one R_{10b}, a C₃-C₆₀ carbocyclic group unsubstituted or substituted with at least one R_{10b}, a C₁-C₆₀ heterocyclic group unsubstituted or substituted with at least one R_{10b}, a C₆-C₆₀ aryloxy group unsubstituted or substituted with at least one R_{10b}, a C₆-C₆₀ arylthio group unsubstituted or substituted with at least one R_{10b}, —Si(Q₄₁)(Q₄₂)(Q₄₃), —N(Q₄₁)(Q₄₂), —B(Q₄₁)(Q₄₂), —C(═O)(Q₄₁), —S(═O)₂(Q₄₁), or —P(═O)(Q₄₁)(Q₄₂),
R_{10b} is:
deuterium, tritium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, tritium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₅₁)(Q₅₂)(Q₅₃), —N(Q₅₁)(Q₅₂), —B(Q₅₁)(Q₅₂), —C(═O)(Q₅₁), —S(═O)₂(Q₅₁), —P(═O)(Q₅₁)(Q₅₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, tritium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₆₁)(Q₆₂)(Q₆₃), —N(Q₆₁)(Q₆₂), —B(Q₆₁)(Q₆₂), —C(═O)(Q₆₁), —S(═O)₂(Q₆₁), —P(═O)(Q₆₁)(Q₆₂), or any combination thereof; or
—Si(Q₇₁)(Q₇₂)(Q₇₃), —N(Q₇₁)(Q₇₂), —B(Q₇₁)(Q₇₂), —C(═O)(Q₇₁), —S(═O)₂(Q₇₁), or —P(═O)(Q₇₁)(Q₇₂), and
Q₄₁ to Q₄₃, Q₅₁ to Q₅₃, Q₆₁ to Q₆₃, and Q₇₁ to Q₇₃ are each independently: hydrogen; deuterium; tritium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, tritium, —F, a cyano group, a C₁-C₆₀ alkyl group, a C₁-C₆₀ alkoxy group, a phenyl group, a biphenyl group, or any combination thereof.

4. The light-emitting device of claim 3, wherein a weight ratio of the first organic compound to the second organic compound is in a range of about 1:10 to about 10:1.

5. The light-emitting device of claim 1, wherein the interlayer further comprises a dopant that is to emit fluorescence or delayed fluorescence.

6. The light-emitting device of claim 1, wherein the emission layer is to emit blue light.

7. An electronic apparatus comprising:
the light-emitting device of claim 1; and
a thin-film transistor electrically connected to the light-emitting device.

8. An electronic equipment comprising the light-emitting device of claim 1, wherein the electronic equipment is at least one of a flat panel display, a curved display, a computer monitor, a medical monitor, a television, a billboard, a light for indoor lighting, a light for outdoor lighting, a light for signaling, a head-up display, a fully transparent display, a partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality display, an augmented reality display, a vehicle, a video wall with multiple displays tiled together, a theater screen, a stadium screen, a phototherapy device, or a signboard.

9. An organic compound represented by Formula 1: 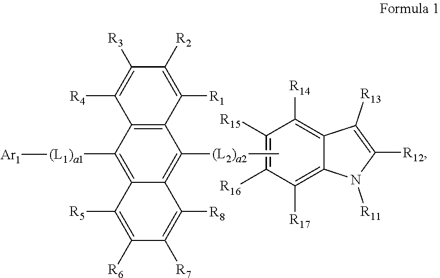
wherein, in Formula 1,
Ar₁ is a C₃-C₆₀ carbocyclic group unsubstituted or substituted with at least one R₁₀ₐ or a C₁-C₆₀ heterocyclic group unsubstituted or substituted with at least one R₁₀ₐ,
L₁ and L₂ are each independently a C₆-C₆₀ arylene group unsubstituted or substituted with at least one R₁₀ₐ or a C₁-C₆₀ heteroarylene group unsubstituted or substituted with at least one R₁₀ₐ,
a1 and a2 are each an integer from 0 to 2,
when a1 is 0, then (L₁)ₐ₁ is a single bond, and when a2 is 0, then (L₂)ₐ₂ is a single bond,
R₁ to R₈ and R₁₁ to R₁₇ are each independently hydrogen, deuterium, tritium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ alkylthio group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂), provided that one selected from among R₁₄ to R₁₇ is a binding site to (L₂)ₐ₂,
R₁₀ₐ is:
deuterium, tritium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, tritium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁n)(Q₁₂), —B(Q₁₁)(Q₁₂), —C(═O)(Q₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, tritium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Qin to Q₁₃, Q₂₁ to Q₂₃, and Q₃₁ to Q₃₃ are each independently: hydrogen; deuterium, tritium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, tritium, —F, a cyano group, a C₁-C₆₀ alkyl group, a C₁-C₆₀ alkoxy group, a phenyl group, a biphenyl group, or any combination thereof.

10. The organic compound of claim 9, wherein, in Formula 1, Ar₁ is a C₆-C₆₀ aryl group or a C₁-C₆₀ heteroaryl group, each unsubstituted or substituted with deuterium, tritium, a cyano group, a C₁-C₆₀ alkyl group, a C₁-C₆₀ alkoxy group, or any combination thereof.

11. The organic compound of claim 9, wherein, in Formula 1, Ar₁ is a phenyl group, a naphthalenyl group, an anthracenyl group, a phenanthrenyl group, a phenalenyl group, a tetracenyl group, a pyrenyl group, a chrysenyl group, a triphenylenyl group, a pentacenyl group, or a perylenyl group, each unsubstituted or substituted with at least one R₁₀ₐ.

12. The organic compound of claim 9, wherein, in Formula 1,
a1 is 0, or
L₁ is a phenylene group unsubstituted or substituted with at least one R₁₀ₐ.

13. The organic compound of claim 9, wherein, in Formula 1, a2 is 0.

14. The organic compound of claim 9, wherein, in Formula 1, R₁₁ to R₁₃ are each independently hydrogen, deuterium, tritium, a C₁-C₆₀ alkyl group unsubstituted or substituted with at least one R₁₀ₐ, a C₆-C₆₀ aryl group unsubstituted or substituted with at least one R₁₀ₐ, or a C₁-C₆₀ heteroaryl group unsubstituted or substituted with at least one R₁₀ₐ.

15. The organic compound of claim 9, wherein, in Formula 1, at least one of R₁₂ or R₁₃ is:
a C₆-C₆₀ aryl group unsubstituted or substituted with at least one R₁₀ₐ or a C₁-C₆₀ heteroaryl group unsubstituted or substituted with at least one R₁₀ₐ.

16. The organic compound of claim 9, wherein the organic compound is represented by Formula 1-1: 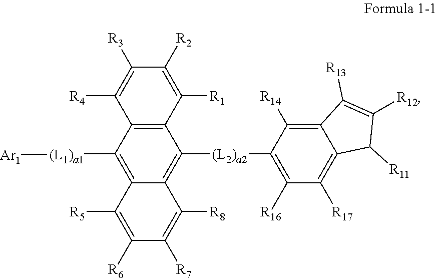
in Formula 1-1,
Ar₁, L₁, L₂, a1, a2, R₁ to R₈, R₁₁ to R₁₄, R₁₆, and R₁₇ being each the same as defined in Formula 1.

17. The organic compound of claim 9, wherein the organic compound comprises at least one deuterium.

18. The organic compound of claim 9, wherein the organic compound satisfies at least one selected from among Conditions 1 to 8:
Condition 1
Ar₁ is a phenyl group substituted with at least one deuterium;
Condition 2
L₁ is a phenylene group substituted with at least one deuterium;
Condition 3
at least one selected from among R₁ to R₈ is deuterium;
Condition 4
L₂ is a phenylene group substituted with at least one deuterium;
Condition 5
at least one selected from among R₁₄ to R₁₇ is deuterium;
Condition 6
R₁₁ is selected from among deuterium, a phenyl group that is substituted with at least one deuterium, and a naphthalenyl group that is substituted with at least one deuterium;
Condition 7
R₁₂ is selected from among deuterium, a phenyl group that is substituted with at least one deuterium, and a naphthalenyl group that is substituted with at least one deuterium; and
Condition 8
R₁₃ is selected from among deuterium, a phenyl group that is substituted with at least one deuterium, and a naphthalenyl group that is substituted with at least one deuterium.

19. The organic compound of claim 9, wherein the organic compound does not comprise any of a benzimidazole group, a benzoxazole group, a benzothiazole group, and a triphenylene group.

20. The organic compound of claim 9, wherein the organic compound is any one selected from among Compounds BH1 to BH54: 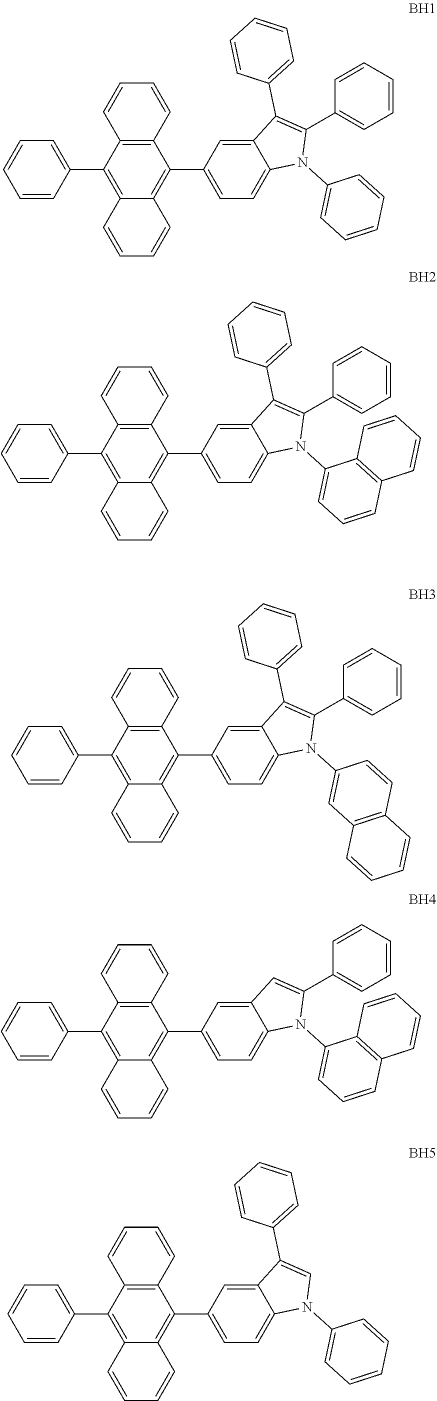 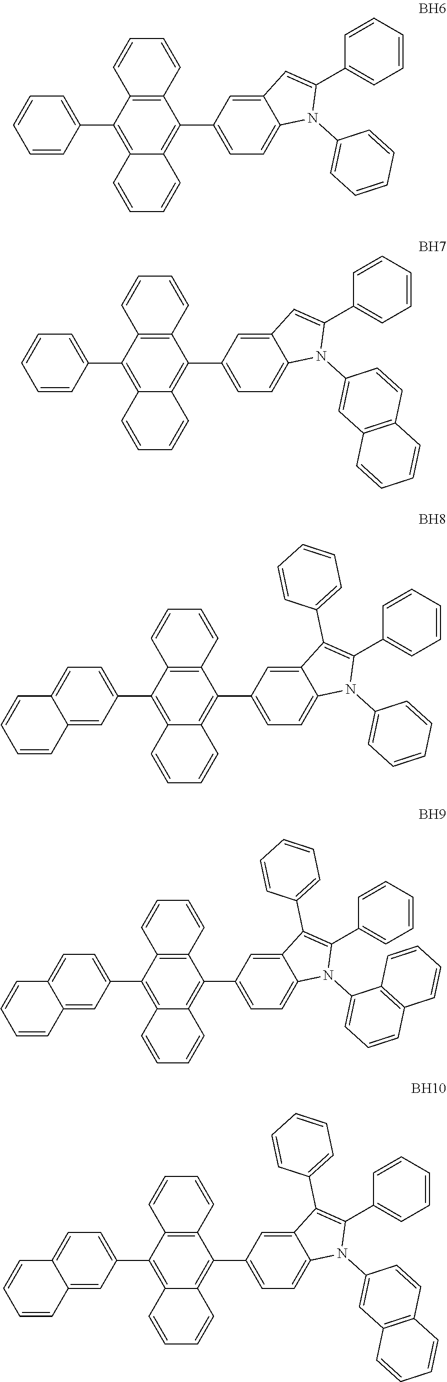 [SMILES: c1ccc(-c2c3ccccc3c(-c3ccc4c(c3)cc(-c3ccccc3)n4-c3ccccc3)c3ccccc23)cc1 ; c1ccc(-c2c3ccccc3c(-c3ccc4c(c3)cc(-c3ccccc3)n4-c3ccc4ccccc4c3)c3ccccc23)cc1 ; c1ccc(-c2c(-c3ccccc3)n(-c3ccccc3)c3ccc(-c4c5ccccc5c(-c5ccc6ccccc6c5)c5ccccc45)cc23)cc1 ; c1ccc(-c2c(-c3ccccc3)n(-c3cccc4ccccc34)c3ccc(-c4c5ccccc5c(-c5ccc6ccccc6c5)c5ccccc45)cc23)cc1 ; c1ccc(-c2c(-c3ccccc3)n(-c3ccc4ccccc4c3)c3ccc(-c4c5ccccc5c(-c5ccc6ccccc6c5)c5ccccc45)cc23)cc1] 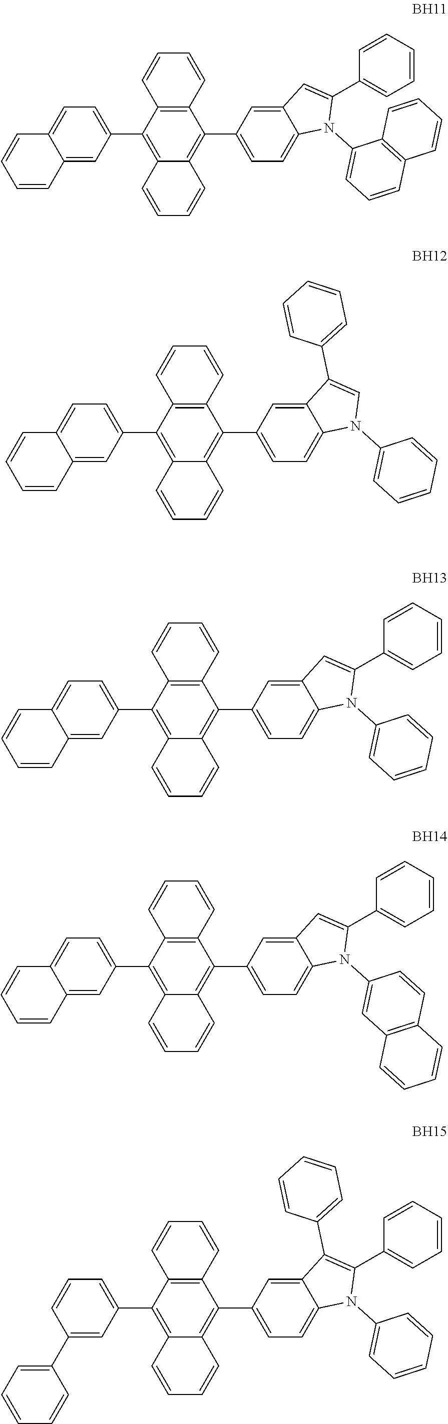 [SMILES: c1ccc(-c2cc3cc(-c4c5ccccc5c(-c5ccc6ccccc6c5)c5ccccc45)ccc3n2-c2cccc3ccccc23)cc1 ; c1ccc(-c2cn(-c3ccccc3)c3ccc(-c4c5ccccc5c(-c5ccc6ccccc6c5)c5ccccc45)cc23)cc1 ; c1ccc(-c2cc3cc(-c4c5ccccc5c(-c5ccc6ccccc6c5)c5ccccc45)ccc3n2-c2ccccc2)cc1 ; c1ccc(-c2cc3cc(-c4c5ccccc5c(-c5ccc6ccccc6c5)c5ccccc45)ccc3n2-c2ccc3ccccc3c2)cc1 ; c1ccc(-c2cccc(-c3c4ccccc4c(-c4ccc5c(c4)c(-c4ccccc4)c(-c4ccccc4)n5-c4ccccc4)c4ccccc34)c2)cc1] 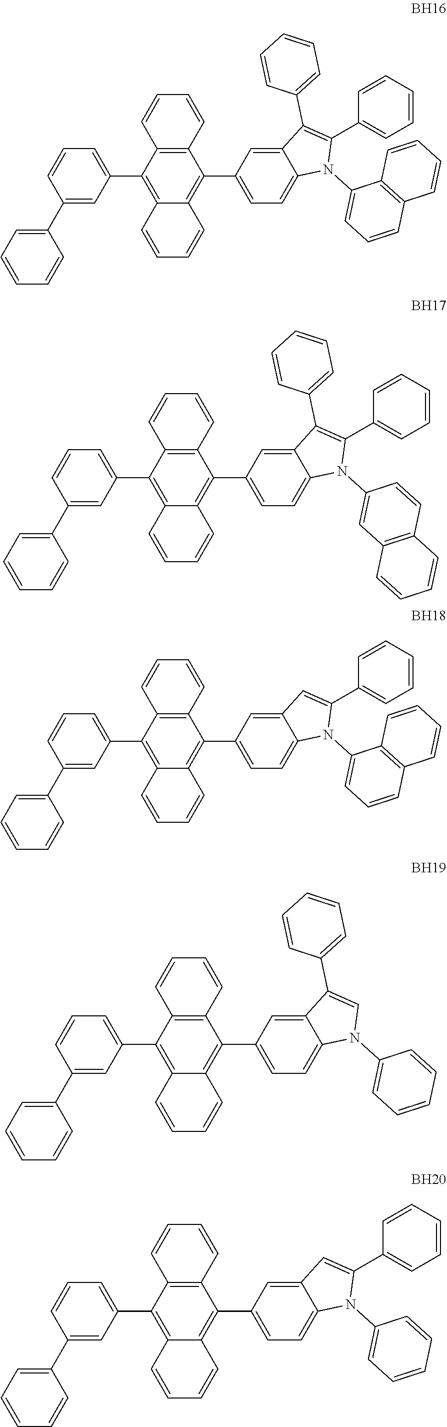 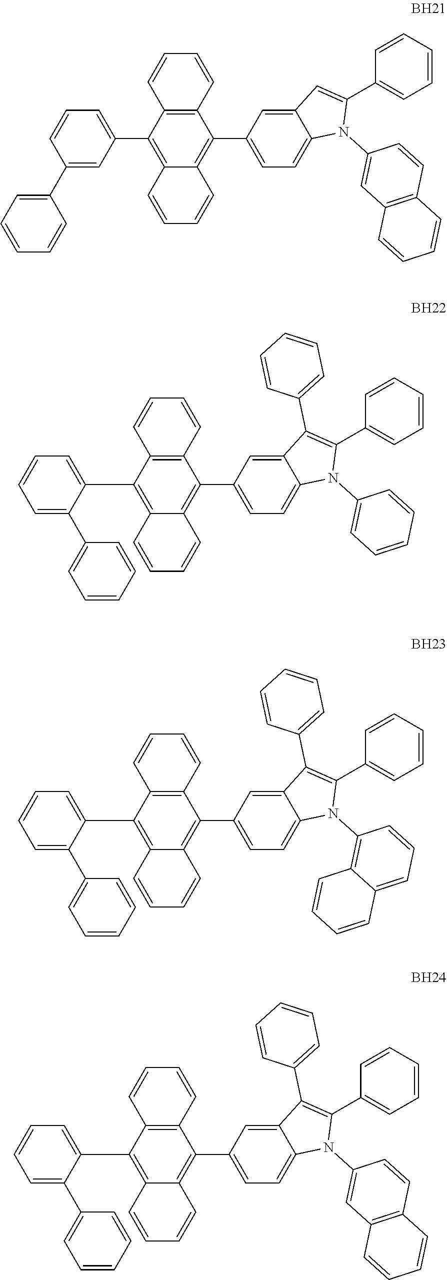 [SMILES: c1ccc(-c2cccc(-c3c4ccccc4c(-c4ccc5c(c4)cc(-c4ccccc4)n5-c4ccc5ccccc5c4)c4ccccc34)c2)cc1 ; c1ccc(-c2ccccc2-c2c3ccccc3c(-c3ccc4c(c3)c(-c3ccccc3)c(-c3ccccc3)n4-c3ccccc3)c3ccccc23)cc1 ; c1ccc(-c2ccccc2-c2c3ccccc3c(-c3ccc4c(c3)c(-c3ccccc3)c(-c3ccccc3)n4-c3cccc4ccccc34)c3ccccc23)cc1 ; c1ccc(-c2ccccc2-c2c3ccccc3c(-c3ccc4c(c3)c(-c3ccccc3)c(-c3ccccc3)n4-c3ccc4ccccc4c3)c3ccccc23)cc1] 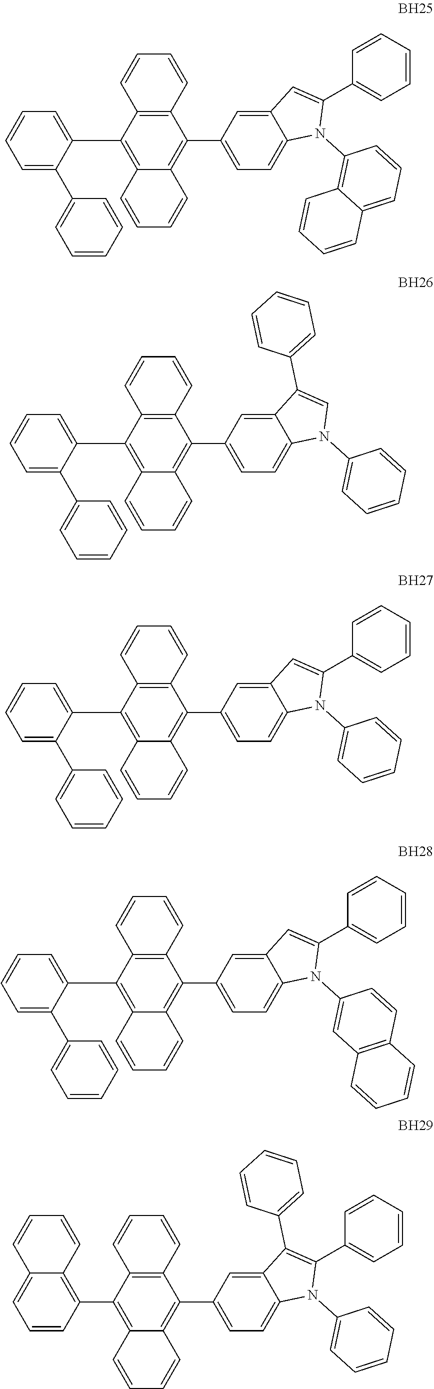 [SMILES: c1ccc(-c2ccccc2-c2c3ccccc3c(-c3ccc4c(c3)cc(-c3ccccc3)n4-c3cccc4ccccc34)c3ccccc23)cc1 ; c1ccc(-c2ccccc2-c2c3ccccc3c(-c3ccc4c(c3)c(-c3ccccc3)cn4-c3ccccc3)c3ccccc23)cc1 ; c1ccc(-c2ccccc2-c2c3ccccc3c(-c3ccc4c(c3)cc(-c3ccccc3)n4-c3ccccc3)c3ccccc23)cc1 ; c1ccc(-c2ccccc2-c2c3ccccc3c(-c3ccc4c(c3)cc(-c3ccccc3)n4-c3ccc4ccccc4c3)c3ccccc23)cc1 ; c1ccc(-c2c(-c3ccccc3)n(-c3ccccc3)c3ccc(-c4c5ccccc5c(-c5cccc6ccccc56)c5ccccc45)cc23)cc1] 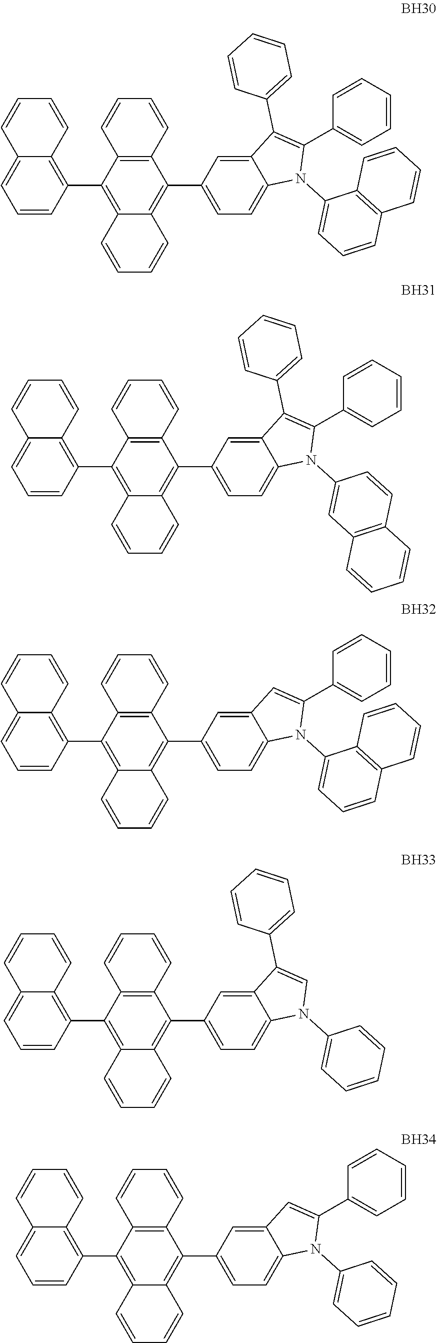 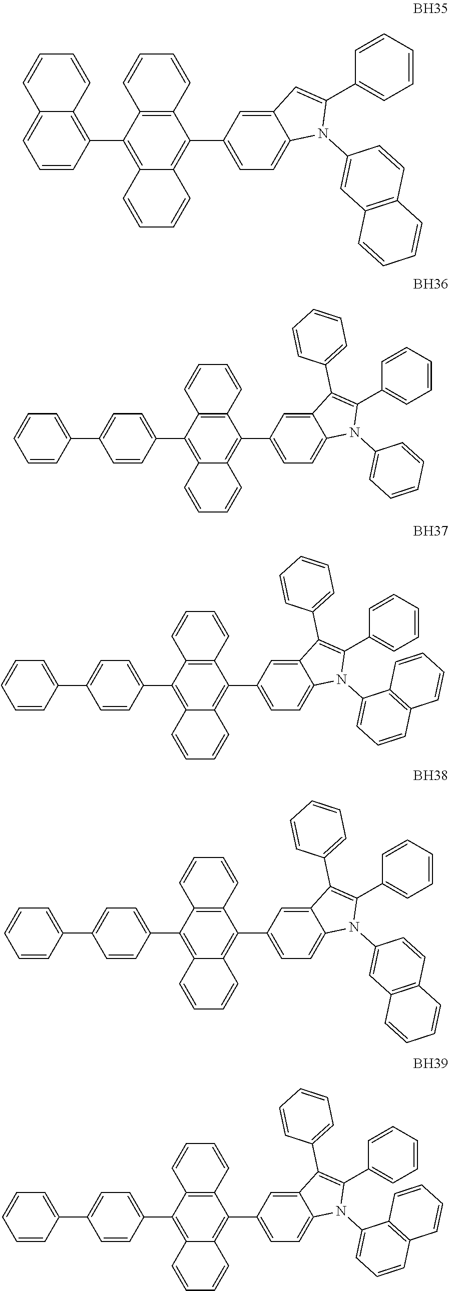 [SMILES: c1ccc(-c2cc3cc(-c4c5ccccc5c(-c5cccc6ccccc56)c5ccccc45)ccc3n2-c2ccc3ccccc3c2)cc1 ; c1ccc(-c2ccc(-c3c4ccccc4c(-c4ccc5c(c4)c(-c4ccccc4)c(-c4ccccc4)n5-c4ccccc4)c4ccccc34)cc2)cc1 ; c1ccc(-c2ccc(-c3c4ccccc4c(-c4ccc5c(c4)c(-c4ccccc4)c(-c4ccccc4)n5-c4cccc5ccccc45)c4ccccc34)cc2)cc1 ; c1ccc(-c2ccc(-c3c4ccccc4c(-c4ccc5c(c4)c(-c4ccccc4)c(-c4ccccc4)n5-c4ccc5ccccc5c4)c4ccccc34)cc2)cc1 ; c1ccc(-c2ccc(-c3c4ccccc4c(-c4ccc5c(c4)c(-c4ccccc4)c(-c4ccccc4)n5-c4cccc5ccccc45)c4ccccc34)cc2)cc1] 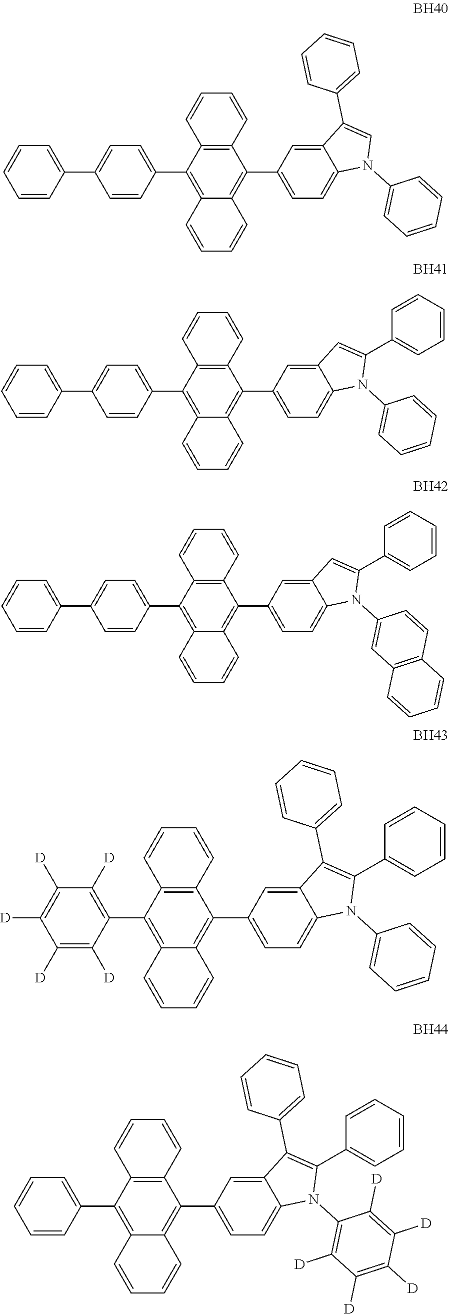 [SMILES: c1ccc(-c2ccc(-c3c4ccccc4c(-c4ccc5c(c4)c(-c4ccccc4)cn5-c4ccccc4)c4ccccc34)cc2)cc1 ; c1ccc(-c2ccc(-c3c4ccccc4c(-c4ccc5c(c4)cc(-c4ccccc4)n5-c4ccccc4)c4ccccc34)cc2)cc1 ; c1ccc(-c2ccc(-c3c4ccccc4c(-c4ccc5c(c4)cc(-c4ccccc4)n5-c4ccc5ccccc5c4)c4ccccc34)cc2)cc1 ; [2H]c1c([2H])c([2H])c(-c2c3ccccc3c(-c3ccc4c(c3)c(-c3ccccc3)c(-c3ccccc3)n4-c3ccccc3)c3ccccc23)c([2H])c1[2H] ; [2H]c1c([2H])c([2H])c(-n2c(-c3ccccc3)c(-c3ccccc3)c3cc(-c4c5ccccc5c(-c5ccccc5)c5ccccc45)ccc32)c([2H])c1[2H]] 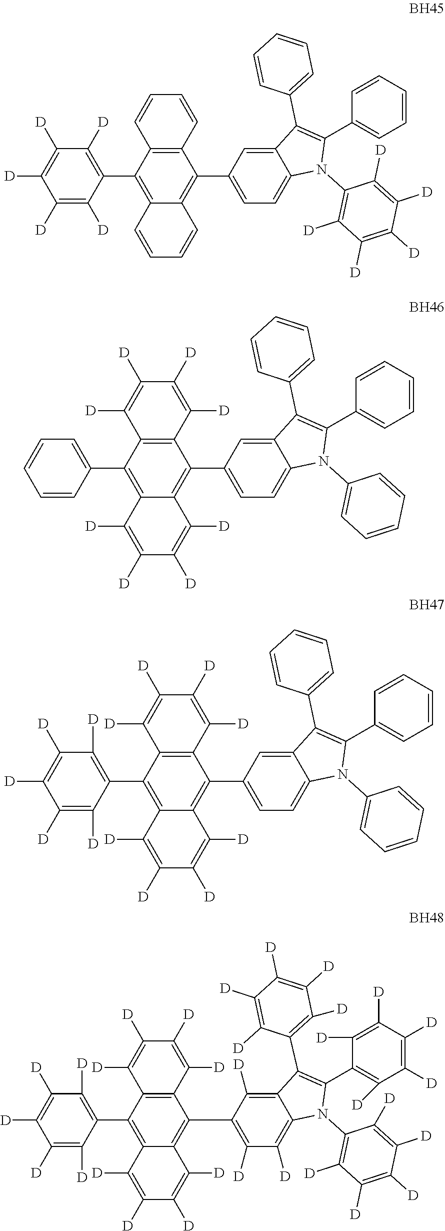 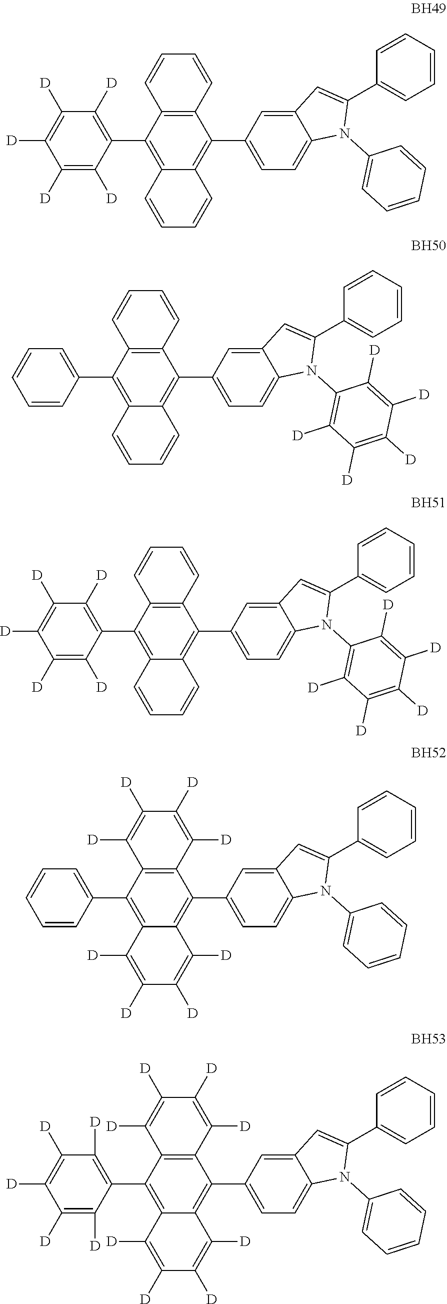 [chemical structure]
